SEQUENCE LISTING (313 sequences)

SEQ ID NO: 1 - protein, 117 aa, synthetic construct
EVQLVESGGGLVQPGGSLRLSCAASGFTFSSYWMHWVRQAPGKGLVWVSRINSDGSSTSYADSVKGRFAISRDNAKNTLYLQMNSLRAEDTAVYYCARALGGWDIDYWGQGTLVTVS

SEQ ID NO: 2 - protein, 7 aa, synthetic construct
GFTFSSY

SEQ ID NO: 3 - skipped

SEQ ID NO: 4 - protein, 11 aa, synthetic construct
ARALGGWDIDY

SEQ ID NO: 5 - protein, 111 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGGVFGGGTKLTVLG

SEQ ID NO: 6 - protein, 8 aa, synthetic construct
SSNIGSNY

SEQ ID NO: 7 - skipped

SEQ ID NO: 8 - protein, 11 aa, synthetic construct
AAWDDSLSGGV

SEQ ID NO: 9 - protein, 111 aa, synthetic construct
QSVLTQPPSVSAAPGQKVTISCSGGSSSIGNNYVSWYQLLPGTAPKLLIYDNNKRPSGIPDRFSGSKSGTSATLGITGLQTGDEADYYCGTWDSGLSAGVFGGGTKLTVLG

SEQ ID NO: 10 - protein, 8 aa, synthetic construct
SSSIGNNY

SEQ ID NO: 11 - skipped

SEQ ID NO: 12 - protein, 11 aa, synthetic construct
GTWDSGLSAGV

SEQ ID NO: 13 - protein, 119 aa, synthetic construct
VQLVESGGGVVQPGRSLRLSCAASGFTFSDYAVHWVRQAPGKGLEWAALISNDGDFKYYADAVKGRFTISRDNSNNTLYLQMNSLRTEDTAVYYCARERGRDKVFPPGYWGQGTLVTVS

SEQ ID NO: 14 - protein, 8 aa, synthetic construct
GFTFSDYA

SEQ ID NO: 15 - protein, 7 aa, synthetic construct
SNDGDFK

SEQ ID NO: 16 - protein, 14 aa, synthetic construct
ARERGRDKVFPPGY

SEQ ID NO: 17 - protein, 111 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGGGTKLTVLG

SEQ ID NO: 18 - protein, 8 aa, synthetic construct
SSNIGNNY

SEQ ID NO: 19 - skipped

SEQ ID NO: 20 - protein, 11 aa, synthetic construct
GTWDSSLSAEV

SEQ ID NO: 21 - protein, 117 aa, synthetic construct
VQLVESGGGVVQPGRSLRLSCEASGFTFRSRAMHWVRQAPGKGLGWVAVIWYDGSNKYYADSVKGRFTISRDNSRNTLYLQMNSLRAEDTAVYYCARDLGSGFSLDYWGQGTLVTVS

SEQ ID NO: 22 - protein, 8 aa, synthetic construct
GFTFRSRA

SEQ ID NO: 23 - protein, 8 aa, synthetic construct
IWYDGSNK

SEQ ID NO: 24 - protein, 12 aa, synthetic construct
ARDLGSGFSLDY

SEQ ID NO: 25 - protein, 111 aa, synthetic construct
QAVVTQEPSLTVSPGGTVTLTCASSTGAVTSAHSPHWFRQKPGQAPRTLIYDTSNKPSWTPARFSGSLLGGKAALTLSGAQPEDEAEYYCLLSNSGVHFLFGGGTRLTVLG

SEQ ID NO: 26 - protein, 9 aa, synthetic construct
TGAVTSAHS

SEQ ID NO: 27 - skipped

SEQ ID NO: 28 - protein, 10 aa, synthetic construct
LLSNSGVHFL

SEQ ID NO: 29 - protein, 119 aa, synthetic construct
EVQLVQSGAEVKKTGSSVKLSCTATGYTFTYRYLHWVRQAPGQALEWMGYITIYNGDTNYAQKFQDRVTISRDMSLSTVYMELSSLTSEDTAMYFCVRSALYGENAFDFWGQGTMVTVS

SEQ ID NO: 30 - protein, 8 aa, synthetic construct
GYTFTYRY

SEQ ID NO: 31 - protein, 8 aa, synthetic construct
ITIYNGDT

SEQ ID NO: 32 - protein, 13 aa, synthetic construct
VRSALYGENAFDF

SEQ ID NO: 33 - protein, 121 aa, synthetic construct
EVQLVESGGGSVQPGGSLRLSCAASGFTFSNYWMHWVRQAPGKGLVWVSRLNIDGSNTFYADSVKGRFTISRDNAKNTLYLQMNSLRAEDTAVYYCVRGGSDSGDSVPFDLWGRGTLVTVS

SEQ ID NO: 34 - skipped

SEQ ID NO: 35 - protein, 8 aa, synthetic construct
LNIDGSNT

SEQ ID NO: 36 - protein, 15 aa, synthetic construct
VRGGSDSGDSVPFDL

SEQ ID NO: 37 - protein, 107 aa, synthetic construct
EIVMTQSPATLSVSPGERATLSCRASQSVSSNLAWYQQNPGQAPRLLIYGASTRATGIPARFSGSGSGTEFTLTISSLQSEDFAVYYCQHYNNWPFTFGQGTKVEIK

SEQ ID NO: 38 - protein, 6 aa, synthetic construct
QSVSSN

SEQ ID NO: 39 - skipped

SEQ ID NO: 40 - protein, 9 aa, synthetic construct
QHYNNWPFT

SEQ ID NO: 41 - protein, 118 aa, synthetic construct
EVQLVESGGGLVQPGGSLRLSCAASGFTFSSYWMHWVRQAPGKGLVWVSRINNDGSSTGYADSVEGRFTISRDNAKNTLYLQINSLRAEDTAVYYCARQDGHYYYSMDVWGQGTTVTV

SEQ ID NO: 42 - protein, 8 aa, synthetic construct
GFTFSSYW

SEQ ID NO: 43 - protein, 8 aa, synthetic construct
INNDGSST

SEQ ID NO: 44 - protein, 13 aa, synthetic construct
ARQDGHYYYSMDV

SEQ ID NO: 45 - protein, 110 aa, synthetic construct
QSALTQPRSVSGSPGQSVTISCTGTSSDVGGYNYVSWYQQHPGKAPKLMIYDVSERPSGVPARFSGSKSGNTASLTISGLQAEDEADYYCCSYAGNFRWVFGGGTKLTVL

SEQ ID NO: 46 - protein, 9 aa, synthetic construct
SSDVGGYNY

SEQ ID NO: 47 - skipped

SEQ ID NO: 48 - protein, 10 aa, synthetic construct
CSYAGNFRWV

SEQ ID NO: 49 - protein, 122 aa, synthetic construct
EVQLVESGGGLVQPGGSLKLSCAASGFSFSGAAMHWVRQSSGRGLEWLGRIRSKTNDYATAYAESLHGRFTISRDDAKNTAYLQMNRLKSEDTAIYYCTTVLSKGDHAVWLGPWGPGTLVTV

SEQ ID NO: 50 - protein, 8 aa, synthetic construct
GFSFSGAA

SEQ ID NO: 51 - protein, 10 aa, synthetic construct
IRSKTNDYAT

SEQ ID NO: 52 - protein, 15 aa, synthetic construct
TTVLSKGDHAVWLGP

SEQ ID NO: 53 - protein, 116 aa, synthetic construct
QPVLTQPPSSSASPGESARLTCTLPSDISVAASDIYWYQQKAGSPPNFLLYDPSDSHKGQDSGVPSRFSGSRDGSANSGFLLISGVQSEDEADYYCMVWPPNTVGVVFGGGTTLTV

SEQ ID NO: 54 - protein, 9 aa, synthetic construct
SDISVAASD

SEQ ID NO: 55 - protein, 7 aa, synthetic construct
DPSDSHK

SEQ ID NO: 56 - protein, 11 aa, synthetic construct
MVWPPNTVGVV

SEQ ID NO: 57 - protein, 110 aa, synthetic construct
QAVVTQEPSLTVSPGGTVTLTCASSTGAVTSGYYPNWFQQKPGQAPRALIYSTSNKHSWTPARFSGSLLGGKAALTLSGVQPEDEAEYYCLLFYGGVHVFGAGTKVTVLG

SEQ ID NO: 58 - protein, 9 aa, synthetic construct
TGAVTSGYY

SEQ ID NO: 59 - skipped

SEQ ID NO: 60 - protein, 9 aa, synthetic construct
LLFYGGVHV

SEQ ID NO: 61 - protein, 115 aa, synthetic construct
EVQLVESGGDLVQPGGSLRLSCAASGFTFSTYWMHWVRQAPGQGLVWVSRINGNGRITNYADSVKGRFTVSRDNAKNTVDLQMNSLRAEDTAVYYCARINEWGDVWGKGTTVTVS

SEQ ID NO: 62 - protein, 8 aa, synthetic construct
GFTFSTYW

SEQ ID NO: 63 - protein, 8 aa, synthetic construct
INGNGRIT

SEQ ID NO: 64 - protein, 9 aa, synthetic construct
ARINEWGDV

SEQ ID NO: 65 - protein, 113 aa, synthetic construct
DIVMTQTPLSLSVTPGQPASISCKSSQSLLHNDGKTFLHWYLQKPGQSPQLLIYEVSSRFSGVPDRFSGSGSGTDFTLKISRVEAEDVGVYYCMQGIHLPPTFGGGTKVEIKR

SEQ ID NO: 66 - protein, 11 aa, synthetic construct
QSLLHNDGKTF

SEQ ID NO: 67 - skipped

SEQ ID NO: 68 - protein, 9 aa, synthetic construct
MQGIHLPPT

SEQ ID NO: 69 - protein, 117 aa, synthetic construct
EVQLVESGGGLVQPGGSLRLSCAASGFTFSSYWMEWVRQAPGKGLVWVSHISSDGSVTRYVDSVKGRFTISRDNAKNTLYLQMNSLRAEDTGVYYCAKDLHWNALDVWGQGTMVTVS

SEQ ID NO: 70 - protein, 8 aa, synthetic construct
ISSDGSVT

SEQ ID NO: 71 - protein, 11 aa, synthetic construct
AKDLHWNALDV

SEQ ID NO: 72 - protein, 109 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIKR

SEQ ID NO: 73 - protein, 10 aa, synthetic construct [sequence omitted]

SEQ ID NO: 74 - protein, 121 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARGVRSGWYADAFDIWGQGTMVTVS

SEQ ID NO: 75 - protein, 8 aa, synthetic construct
INSDGSST

SEQ ID NO: 76 - protein, 15 aa, synthetic construct
ARGVRSGWYADAFDI

SEQ ID NO: 77 - protein, 119 aa, synthetic construct
EVQLVESGGGLVQPGGSLGLSCAASGFTFSNYWMHWVRQAPGKGLVWVSRINSDGSDTSYADSVKGRFTISRDNAENTLYLHMSSLRAEDTAVYYCARELGYGDYGMDVWGKGTTVTVS

SEQ ID NO: 78 - protein, 8 aa, synthetic construct
GFTFSNYW

SEQ ID NO: 79 - protein, 8 aa, synthetic construct
INSDGSDT

SEQ ID NO: 80 - protein, 13 aa, synthetic construct
ARELGYGDYGMDV

SEQ ID NO: 81 - protein, 112 aa, synthetic construct
QSVLTQPPSASGTPGQRVTISCSGSSSNIGSHSVIWYQQLPGTAPKLLVYSDNQRPSGVPDRFSGSKSGTSASLAISGLQSEDEADYYCAAWDDSLNHLHVVFGGGTKLTVL

SEQ ID NO: 82 - protein, 8 aa, synthetic construct
SSNIGSHS

SEQ ID NO: 83 - skipped

SEQ ID NO: 84 - protein, 13 aa, synthetic construct
AAWDDSLNHLHVV

SEQ ID NO: 85 - protein, 116 aa, synthetic construct
EVQLVESGGGLVQPGGSLRLSCAASGFIFSNYWMHWVRQAPGEGLVWVSRINKDGSSTFYADSVKGRFTISRDNAKNTLYLQMNSLRAEDTAVYYCTRDFDFWSGYWGQGTLVTVS

SEQ ID NO: 86 - protein, 8 aa, synthetic construct
GFIFSNYW

SEQ ID NO: 87 - protein, 8 aa, synthetic construct
INKDGSST

SEQ ID NO: 88 - protein, 10 aa, synthetic construct
TRDFDFWSGY

SEQ ID NO: 89 - protein, 110 aa, synthetic construct
QSALTQPASVSGSPGQSITISCTGTSSDVGGCEYVSWYQQHPGKVPKLIIYEVSNRPSGVSNRFSGSKSGNTASLTVSGLQAEDEADYFCSSCTTSGSYVFGAGTKVTVV

SEQ ID NO: 90 - protein, 9 aa, synthetic construct
SSDVGGCEY

SEQ ID NO: 91 - protein, 10 aa, synthetic construct
SSCTTSGSYV

SEQ ID NO: 92 - protein, 125 aa, synthetic construct
EVQLVESGGGVVQPGESLRLSCAASGVTFSSYWMHWVRQVPGKGLVWVARINIDGTSTTYADCVKGRFTISRDNAKNTLYLQMSSLRAEDTAVYYCATERGLLSGGRWHSSHFDYWGQGTLVTVS

SEQ ID NO: 93 - protein, 8 aa, synthetic construct
GVTFSSYW

SEQ ID NO: 94 - protein, 8 aa, synthetic construct
INIDGTST

SEQ ID NO: 95 - protein, 19 aa, synthetic construct
ATERGLLSGGRWHSSHFDY

SEQ ID NO: 96 - protein, 109 aa, synthetic construct
DIQMTQSPSTLSASVGDRVTITCRASQSISSWLAWYQQKPGKAPKVLIYKTSSLESGVPSRFSGSGSGTEFTLTISSLQPDDFATYYCQQYDSSSLTWTFGQGTKVEIK

SEQ ID NO: 97 - protein, 6 aa, synthetic construct
QSISSW

SEQ ID NO: 98 - skipped

SEQ ID NO: 99 - protein, 11 aa, synthetic construct
QQYDSSSLTWT

SEQ ID NO: 100 - protein, 119 aa, synthetic construct
EVQLVESGGGLVQPGGSLRLSCAASGFTFSRFWMHWVRQVPGKGLVWISRINSDATSSSYADSVKGRFTISRDNAKNTLFLQMNSLRAEDTAVYYCATSNTALVYLPENWGQGTLVTVS

SEQ ID NO: 101 - protein, 8 aa, synthetic construct
GFTFSRFW

SEQ ID NO: 102 - protein, 8 aa, synthetic construct
INSDATSS

SEQ ID NO: 103 - protein, 13 aa, synthetic construct
ATSNTALVYLPEN

SEQ ID NO: 104 - protein, 107 aa, synthetic construct
EIVLTQSPATLSLSPGERATLSCRASQSVSDYLAWYQQKPGQAPRLLIYDASNRATGIPARFSGGGSGTDFTLTISSLEPEDFAVYYCQQRSYWPPTFGQGTKVEIK

SEQ ID NO: 105 - protein, 6 aa, synthetic construct
QSVSDY

SEQ ID NO: 106 - skipped

SEQ ID NO: 107 - protein, 9 aa, synthetic construct
QQRSYWPPT

SEQ ID NO: 108 - protein, 111 aa, synthetic construct
QSALTQPPSASGSPGQSVTITCTGTSSDVGGYNYVSWYQQHPGKAPKLMIYEVSKRPSGVPDRFSGSKSGNTASLTVSGLQAEDEADYHCSSFAGDNNSPVFGGGTKLTVL

SEQ ID NO: 109 - protein, 11 aa, synthetic construct
SSFAGDNNSPV

SEQ ID NO: 110 - protein, 119 aa, synthetic construct
EVQLVESGGGLIHPGGSLRLSCAASGFTFSSYWMHWVRQAPGKGLVWVSHIKSDGSNTIYADSVKGRFTISRDNAKNTLYLQMNSLRAEDTAVYYCGRGRSLTPRSAIDYWGQGTLVTV

SEQ ID NO: 111 - protein, 119 aa, synthetic construct; identical to SEQ ID NO: 110

SEQ ID NO: 112 - protein, 14 aa, synthetic construct
GRGRSLTPRSAIDY

SEQ ID NO: 113 - protein, 115 aa, synthetic construct
LVESGGGLVQPGGSLRLSCAASGFTFSSYAMHWVRQAPGKGLEFASAISSDGGTTYYANSVKGRFTISRDNSKNTLYLQMGSLRDEDMAVYYCARDDLSTSWDLDYWGQGTLVTV

SEQ ID NO: 114 - protein, 8 aa, synthetic construct
ISSDGGTT

SEQ ID NO: 115 - protein, 13 aa, synthetic construct
ARDDLSTSWDLDY

SEQ ID NO: 116 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTISSTNVFGTGTKVTVL

SEQ ID NO: 117 - protein, 10 aa, synthetic construct
SSYTISSTNV

SEQ ID NO: 118 - protein, 110 aa, synthetic construct
QSALTQPASMSGSPGQSITISCTGTSSDVGAHNFISWYQQHPGKAPKLMIFEVNKRPSGVSNRFSGSKSGNTASLTISGLQAEDEADYFCCSYAGYSTWVFGGGTKLTVL

SEQ ID NO: 119 - protein, 9 aa, synthetic construct
SSDVGAHNF

SEQ ID NO: 120 - skipped

SEQ ID NO: 121 - protein, 9 aa, synthetic construct
SYAGYSTWV

SEQ ID NO: 122 - protein, 119 aa, synthetic construct
EVQLVESGGGVVQPGGSLRLSCEVSGFIFSSYWMHWVRQVAGKGLVWVSRINSDGSSTSYADSVKGRFTISRDNAKNTLNLQMNFLRAEDTAVYYCARGGDHGDYGFFESWGQGTLVTV

SEQ ID NO: 123 - protein, 8 aa, synthetic construct
GFIFSSYW

SEQ ID NO: 124 - protein, 14 aa, synthetic construct
ARGGDHGDYGFFES

SEQ ID NO: 125 - protein, 117 aa, synthetic construct
EVQLVESGGGLVQPGGSLRLSCAASGFTFSNHWMHWVRQAPGKGLLWVSRIDTGGSTTNYADSVKGRFTISRDSAKNTVYLQMNSLRAEDTAVYYCARGGLFYYGMDVWGQGTTVTV

SEQ ID NO: 126 - protein, 8 aa, synthetic construct
GFTFSNHW

SEQ ID NO: 127 - protein, 8 aa, synthetic construct
IDTGGSTT

SEQ ID NO: 128 - protein, 12 aa, synthetic construct
ARGGLFYYGMDV

SEQ ID NO: 129 - protein, 7 aa, synthetic construct
QTSSSTS

SEQ ID NO: 130 - protein, 10 aa, synthetic construct
QQFSGSPAYT

SEQ ID NO: 131 - protein, 113 aa, synthetic construct
DIVMTQTPLSLPVTPGEPASISCRSSQSLLDSADGNTYLDWYLQKPGQSPQLLIHTLSHRASGVPDRFSGSGSGTDFTLKISRVEAEDVGVYYCMQRIAFPVTFGPGTKVDIK

SEQ ID NO: 132 - protein, 12 aa, synthetic construct
QSLLDSADGNTY

SEQ ID NO: 133 - skipped

SEQ ID NO: 134 - protein, 9 aa, synthetic construct
MQRIAFPVT

SEQ ID NO: 135 - protein, 115 aa, synthetic construct
EVQLVESGGGLVQPGGSLRLSCAASGFTFSNYWMHWVRQAPGEGLEWVSRINSDGNKVSYADSVRGRFTISRDNAKNTLYLQMNSLTGEDTAVYYCARSNWGSADYWGQGTLVTV

SEQ ID NO: 136 - protein, 8 aa, synthetic construct
INSDGNKV

SEQ ID NO: 137 - protein, 10 aa, synthetic construct
ARSNWGSADY

SEQ ID NO: 138 - protein, 125 aa, synthetic construct
EVQLVESGGGSVQPGGSLRLSCAASGFTFSRYWMHWVRQAPGEGLVWVSRINSDETDKLYADSVKGRFSIFRDNAKNTLYLQMNRLRAEDTAVYYCARDREDVVVGPATQHTIFNSWGQGTLVTV

SEQ ID NO: 139 - protein, 8 aa, synthetic construct
GFTFSRYW

SEQ ID NO: 140 - protein, 8 aa, synthetic construct
INSDETDK

SEQ ID NO: 141 - protein, 10 aa, synthetic construct
ARDREDVVVG

SEQ ID NO: 142 - protein, 111 aa, synthetic construct
QSALTQPASVSGSPGQSITISCTGTSSDVGSSNFVSWYQQHPGKAPKLILYEVSKRPAGVSSRFSGSRSGNTASLTISGAQAEDEADYSCCSTSSVGTLYVFGTGTKVTVL

SEQ ID NO: 143 - protein, 7 aa, synthetic construct
DVGSSNF

SEQ ID NO: 144 - protein, 11 aa, synthetic construct
CSTSSVGTLYV

SEQ ID NO: 145 - protein, 110 aa, synthetic construct
QSVLTQPPSVSAAPGQKVTISCSGSNYNIGNNYVSWYQRLPGTAPKLLIYDNNKRPSGIPDRFSGSKSGTSATLGITGLQTGDEADYYCGTWDSSLRAGVFGGGTKLTVL

SEQ ID NO: 146 - protein, 8 aa, synthetic construct
NYNIGNNY

SEQ ID NO: 147 - protein, 11 aa, synthetic construct
GTWDSSLRAGV

SEQ ID NO: 148 - protein, 118 aa, synthetic construct
EVQLVESGGGLVQPGGSLRLSCAASGFTFSRHWMHWVRQAPGKGLVWVSRINSDGSSTSNADSVKGRITISRDNGKNTLYLQMNSLRAEDTAVYYCAREIASGTDAFDIWGQGTMVTV

SEQ ID NO: 149 - protein, 8 aa, synthetic construct
GFTFSRHW

SEQ ID NO: 150 - protein, 13 aa, synthetic construct
AREIASGTDAFDI

SEQ ID NO: 151 - protein, 122 aa, synthetic construct
EVQLVESGGGLVKPGESLRLSCVGSGFTLTNAWMIWVRQTSGKGLEWVGRIKSKIDGGAIDYGAPVKGRFTISRDDTKNTVYLQMNSLQTDDTGVYFCTTDRYSTGYYGMDDYWGQGTLVTV

SEQ ID NO: 152 - protein, 8 aa, synthetic construct
GFTLTNAW

SEQ ID NO: 153 - protein, 9 aa, synthetic construct
IKSKIDGGA

SEQ ID NO: 154 - protein, 15 aa, synthetic construct
TTDRYSTGYYGMDDY

SEQ ID NO: 155 - protein, 105 aa, synthetic construct
SYELTQPPSVSVSPGQTATITCSGDKLGDKYSFWYQQKPGQSPVVVIYQDSKRPSGIPERFSGSNSGNTATLTISGTQAMDEADYYCQVWDTDSAVFGTGTRVTV

SEQ ID NO: 156 - protein, 6 aa, synthetic construct
KLGDKY

SEQ ID NO: 157 - skipped

SEQ ID NO: 158 - protein, 9 aa, synthetic construct
QVWDTDSAV

SEQ ID NO: 159 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 160 - protein, 6 aa, synthetic construct
QSISSY

SEQ ID NO: 161 - skipped

SEQ ID NO: 162 - protein, 9 aa, synthetic construct
QQSYSTPYT

SEQ ID NO: 163 - protein, 113 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDTISRYWGQGTLVTV

SEQ ID NO: 164 - protein, 8 aa, synthetic construct
GFTFSSYS

SEQ ID NO: 165 - protein, 8 aa, synthetic construct
ISSSSSYI

SEQ ID NO: 166 - protein, 8 aa, synthetic construct
ARDTISRY

SEQ ID NO: 167 - protein, 122 aa, synthetic construct
EVQLVESGGGLVKPGGSLRLSCAASGFTFSSYSMNWVRQAPGKGLEWVSSISGSSSYMHYAESVKGRFTISRDNAKNSLYLQMNSLRAEDTAVYYCASTGIITYYYASGVPDYWGQGTLVTV

SEQ ID NO: 168 - protein, 8 aa, synthetic construct
ISGSSSYM

SEQ ID NO: 169 - protein, 16 aa, synthetic construct
STGIITYYYASGVPDY

SEQ ID NO: 170 - protein, 110 aa, synthetic construct
QSVLTQPPSASGTPGQRVTMSCSGSSSNIGRNYVYWYQQLPGTAPKLLIYRNNQRPSGVPDRFSGSKSGTSVSLAISGLRSEDEADYYCAAWDDSLSGVVFGGGTKLTVL

SEQ ID NO: 171 - protein, 8 aa, synthetic construct
SSNIGRNY

SEQ ID NO: 172 - protein, 11 aa, synthetic construct
AAWDDSLSGVV

SEQ ID NO: 173 - protein, 107 aa, synthetic construct
EIVLTQSPGTLSLSPGERATLSCRASQSVSSNLAWYQQKPGQAPRVLIYGASSRATGIPDRFSGSRSGTDFTLTISRLEPEDFAVYYCQRYDNSPRTFGQGTKVEIK

SEQ ID NO: 174 - protein, 9 aa, synthetic construct
QRYDNSPRT

SEQ ID NO: 175 - protein, 119 aa, synthetic construct
EVQLVESGGGLVKPGGSLRLSCAASGFTFINAWMNWVRQAPGKGLEWVGLIKSKTDGGTIDYAAPVKGRFTISRDDSEKMLYLQMDSLKTEDTAVYYCTTMYGWKDARDYWGQGTLVTV

SEQ ID NO: 176 - protein, 8 aa, synthetic construct
GFTFINAW

SEQ ID NO: 177 - protein, 9 aa, synthetic construct
IKSKTDGGT

SEQ ID NO: 178 - protein, 12 aa, synthetic construct
TTMYGWKDARDY

SEQ ID NO: 179 - protein, 108 aa, synthetic construct
EIVLTQSPGTLSLSPGERATLSCRASQSVSSSYLAWYQQKPGQAPRFLIYGASSRATGIPDRFSGSGSGTDFTLTISRLEPEDFAVYYCQQYGSSPYTFGQGTKVEIK

SEQ ID NO: 180 - protein, 7 aa, synthetic construct
QSVSSSY

SEQ ID NO: 181 - protein, 9 aa, synthetic construct
QQYGSSPYT

SEQ ID NO: 182 - protein, 121 aa, synthetic construct
EVQLVESGGGLVQPGGSLRLSCAASGFTFSSYWMHWVRQAPGKGLVWVSRIKSDGSSTSYADSVKGRFTISRDNAKNTLYLQMNSLRAEDTAVYYCARGGGSSNWYPGFFDYWGQGTLVTV

SEQ ID NO: 183 - protein, 8 aa, synthetic construct
IKSDGSST

SEQ ID NO: 184 - protein, 16 aa, synthetic construct
ARGGGSSNWYPGFFDY

SEQ ID NO: 185 - protein, 118 aa, synthetic construct
EVQLLESGGGLVQPGGSLRLSCVASGFTFSSYAMSWVRQAPGKGLEWVSSISASGGSTYYADSVKGRFTISRDNSKNTLYLQMNSLRAEDTAVYYCGNWPEGFPAYFHYWGQGTLVTV

SEQ ID NO: 186 - protein, 8 aa, synthetic construct
GFTFSSYA

SEQ ID NO: 187 - protein, 8 aa, synthetic construct
ISASGGST

SEQ ID NO: 188 - protein, 13 aa, synthetic construct
GNWPEGFPAYFHY

SEQ ID NO: 189 - protein, 107 aa, synthetic construct
EIVMTQSPATLSVSPGERVTLSCRASQSVTSELAWYQQKPGQAPRLLIYDASTGATGIPARFSGSGSGTDFTLTISSLQSEDFAVYYCQQHNNWPLTFGGGTKVEIK

SEQ ID NO: 190 - protein, 6 aa, synthetic construct
QSVTSE

SEQ ID NO: 191 - protein, 9 aa, synthetic construct
QQHNNWPLT

SEQ ID NO: 192 - protein, 122 aa, synthetic construct
EVQLVESGGGLVQPGGSLRLSCAASGFTFSSYWMHWVRQAPGKGLVWVSRIKSDGRSISYADSVKGRFTISRDNAKNTLYLQMNSLRAEDTAVYYCARDPHGTAAPPRDAFDIWGQGTMVTV

SEQ ID NO: 193 - protein, 8 aa, synthetic construct
IKSDGRSI

SEQ ID NO: 194 - protein, 17 aa, synthetic construct
ARDPHGTAAPPRDAFDI

SEQ ID NO: 195 - skipped

SEQ ID NO: 196 - protein, 110 aa, synthetic construct
QSALTQPASVSGSPGQSITISCTGTSGDVGSYNLVSWYQQYPGKAPKLMIYEVDKRPSGVSNRFSGSKSGNTASLTISGLRAEDEAHYHCFSYAGSLTLVFGGGTKLTVL

SEQ ID NO: 197 - protein, 9 aa, synthetic construct
SGDVGSYNL

SEQ ID NO: 198 - skipped

SEQ ID NO: 199 - protein, 10 aa, synthetic construct
FSYAGSLTLV

SEQ ID NO: 200 - protein, 110 aa, synthetic construct
QSVLTQPPSVSGTPGQRVTISCSGSSSNIGSKTANWYQKLPGTAPKLLIYSNNQRPSGVPDRFSGSKSGTSASLAISGLQSEDEAGYYCTAWDDSLNGPVFGGGTKLTVL

SEQ ID NO: 201 - protein, 7 aa, synthetic construct
SSNIGSK

SEQ ID NO: 202 - skipped

SEQ ID NO: 203 - protein, 11 aa, synthetic construct
TAWDDSLNGPV

SEQ ID NO: 204 - protein, 115 aa, synthetic construct
EVQLVESGGGLVQPGGSLRLSCGASGFTVSGKYMTWARQAPEKGLEWVSAIYRGGGTYYADSVKGRFTISRDNSKNMLYLQMNSLRAEDTAVYYCGGSVMVSATDYWGQGTLVTV

SEQ ID NO: 205 - protein, 8 aa, synthetic construct
GFTVSGKY

SEQ ID NO: 206 - protein, 7 aa, synthetic construct
IYRGGGT

SEQ ID NO: 207 - protein, 11 aa, synthetic construct
GGSVMVSATDY

SEQ ID NO: 208 - protein, 4 aa, synthetic construct
AASS

SEQ ID NO: 209 - protein, 113 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDQISGYWGQGTLVTV

SEQ ID NO: 210 - protein, 8 aa, synthetic construct
ARDQISGY

SEQ ID NO: 211 - protein, 115 aa, synthetic construct
EVQLVESGGGLVQPGGSLRLSCVASGFTFSSYWMHWVRQAPGKGLVWVSRIYPDGTTTANYADSVKGRFTISRDNAKNTVYLQMNSLRAEDTAVYYCARDLRESDYWGQGTLVTV

SEQ ID NO: 212 - protein, 9 aa, synthetic construct
IYPDGTTTA

SEQ ID NO: 213 - protein, 9 aa, synthetic construct
ARDLRESDY

SEQ ID NO: 214 - protein, 110 aa, synthetic construct
QSALTQPASVSGSPGQSITISCTGTSSDVGNYNYVSWHQQHPGKAPKLMIYDVSNRPSGVSNRFSGSKSGNTASLTISGLQAEDEADYYCNSYTTYSTHVFGTGTKVTVL

SEQ ID NO: 215 - protein, 9 aa, synthetic construct
SSDVGNYNY

SEQ ID NO: 216 - protein, 10 aa, synthetic construct
NSYTTYSTHV

SEQ ID NO: 217 - DNA, 351 bp, synthetic construct
  source 1..351: mol_type = other DNA
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcgccatctccagagacaacgccaagaacacgctgtatctgcaaatgaacagtctgagagccgaggacacggctgtgtattactgtgcaagagctttgggtggctgggatattgactactggggccagggaaccctggtcaccgtctcg

SEQ ID NO: 218 - DNA, 333 bp, synthetic construct
  source 1..333: mol_type = other DNA
cagtctgtgctgacgcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtggagtgttcggcggagggaccaagctgaccgtcctaggt

SEQ ID NO: 219 - DNA, 333 bp, synthetic construct
  source 1..333: mol_type = other DNA
cagtctgtgctgacgcagccgccctcagtgtctgcggccccgggacagaaggtcaccatctcctgctctggaggcagctcctccattgggaataattatgtatcctggtaccaactgctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcggcctgagtgctggggtgttcggcggagggaccaagctgaccgtcctaggt

SEQ ID NO: 220 - DNA, 357 bp, synthetic construct
  source 1..357: mol_type = other DNA
gtgcagctggtggagtctgggggaggcgtggtccagcctgggaggtccctgagactctcctgtgcagcctctggattcaccttcagtgactatgctgtgcactgggtccgccaggctccaggcaaggggctggagtgggcggcacttatatcaaatgatggagattttaaatattacgcagacgccgtgaagggccgattcaccatctccagagacaattccaacaacacgctgtatctacaaatgaacagcctgagaactgaggacacggctgtgtattactgtgcgagagaaagagggagggataaagtattcccgccgggctactggggccagggaaccctggtcaccgtctcg

SEQ ID NO: 221 - DNA, 333 bp, synthetic construct
  source 1..333: mol_type = other DNA
cagtctgtgctgactcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctgaggtgttcggcggagggaccaagctgaccgtcctaggt

SEQ ID NO: 222 - DNA, 351 bp, synthetic construct
  source 1..351: mol_type = other DNA
gtgcagctggtggagtctgggggaggcgtggtccagcctgggaggtccctgagactctcctgtgaagcgtctggattcaccttcaggagtcgtgccatgcactgggtccgccaggctccaggcaaggggctggggtgggtggcagttatatggtacgatggaagtaataaatactatgcagactccgtgaagggccgattcaccatctccagagacaattccaggaacacgctgtatctgcaaatgaacagcctgagagccgaggacacggctgtatattactgtgcgagagatttgggtagtggtttttcccttgactactggggccagggaaccctggtcaccgtctcg

SEQ ID NO: 223 - DNA, 335 bp, synthetic construct
  source 1..335: mol_type = other DNA
caggctgtggtgactcaggagccctcactgactgtgtccccaggagggacagtcactctcacctgtgcctccagcactggagctgtcaccagtgctcattctccccactggttccgacagaagcctggccaagcccccaggacactgatttatgatacatccaacaaaccgtcctggacacctgcccggttctcaggctccctccttgggggcaaagctgccctgaccctttcgggcgcgcagcctgaggatgaggctgagtattactgcttgctctccaatagtggagtccattttctattcggcggggggaccaggttgaccgtcctaggtca

SEQ ID NO: 224 - DNA, 357 bp, synthetic construct
  source 1..357: mol_type = other DNA
gaggtgcagctggtgcagtctggggctgaggtgaagaagactgggtcctcagtgaagctctcctgcacggctaccggatacactttcacctatcgctacctgcactgggtgcgacaggcccccggacaagcacttgagtggatgggctacataacaatttacaatggtgacaccaattacgcacagaaattccaggacagagtcaccatttccagggacatgtctctgagcacagtctacatggagctgagcagcctgacatcagaggacacggccatgtatttctgtgtaagatccgcattgtatggggaaaatgcttttgatttttggggccaagggacaatggtcaccgtctca

SEQ ID NO: 225 - DNA, 363 bp, synthetic construct
  source 1..363: mol_type = other DNA
gaggtgcagctggtggagtcggggggaggctcagttcagccgggggggtccctgagactctcctgtgcagcctctggattcaccttcagtaactactggatgcactgggtccgccaagctccagggaaggggctggtgtgggtctcacgtcttaatattgatgggagtaacacattctacgcggactccgtcaagggccgattcaccatctccagagacaacgccaagaacacgctgtatctgcaaatgaacagtctgagagccgaggacacggctgtgtattactgtgtaagaggagggagtgactccggtgactccgttcccttcgatctctggggccgtggcaccctggtcaccgtctcg

SEQ ID NO: 226 - DNA, 322 bp, synthetic construct
  source 1..322: mol_type = other DNA
gaaatagtgatgacgcagtctccagccaccctgtctgtgtctccaggggaaagggccaccctctcctgcagggccagtcagagtgttagcagcaacttagcctggtaccagcagaaccctggccaggctcccaggctcctcatctatggtgcatccaccagggccactggtatcccagccaggttcagtggcagtggatctgggacagagttcactctcaccatcagcagcctgcagtctgaagattttgcagtttattactgtcagcactataataactggccgttcacttttggccaggggaccaaggtggaaatcaaac

SEQ ID NO: 227 - DNA, 356 bp, synthetic construct
  source 1..356: mol_type = other DNA
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaataatgatgggagtagcacaggctacgcggactctgtggagggccgattcaccatctccagagacaacgccaagaacacgctgtatctgcaaatcaacagtctgagagccgaggacacggctgtgtattactgtgcaagacaagatggtcactactactacagtatggacgtctggggccaagggaccacggtcaccgtctc

SEQ ID NO: 228 - DNA, 332 bp, synthetic construct
  source 1..332: mol_type = other DNA
cagtctgccctgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcagtgagcggccctcaggggtccctgctcgcttctctggctccaagtctggcaacacggcctccctgaccatctctgggctccaggctgaggatgaggctgattattactgctgctcatatgcaggcaacttccgttgggtgttcggcggagggaccaagctgaccgtcctagg

SEQ ID NO: 229 - DNA, 368 bp, synthetic construct
  source 1..368: mol_type = other DNA
gaggtgcagctggtggagtctgggggaggcttggtccagccgggggggtccctgaaactctcctgtgcagcctctgggttcagtttcagtggcgctgcgatgcactgggtccgccagtcctccgggagagggcttgagtggcttggccgtattagaagcaaaactaacgactatgcgacagcatatgcagagtcgctgcacggcaggttcaccatctccagagatgatgcaaagaacacggcgtatctacaaatgaacaggctgaaaagcgaggacacggccatatattattgtacaaccgtcttgagtaagggagatcatgcggtctggttgggcccctggggcccgggaaccctggtcaccgtctc

SEQ ID NO: 230 - DNA, 348 bp, synthetic construct
  source 1..348: mol_type = other DNA
cagcctgtgctgactcagccaccttcctcctccgcgtctcctggagaatccgccagactcacctgcaccttgcccagtgacatcagtgttgctgcgtctgacatttattggtatcaacagaaggcagggagccctcccaactttctcctctacgacccgtcagactcacataagggccaggactctggagtccccagccgcttctctggatccagagatggatcagccaattcagggtttttactgatttccggggtccagtctgaggatgaggctgactattactgcatggtctggccacccaatactgtgggtgtcgtcttcggcggagggaccactctgaccgtc

SEQ ID NO: 231 - DNA, 330 bp, synthetic construct
  source 1..330: mol_type = other DNA
caggctgtggtgactcaggagccctcactgactgtgtccccaggagggacagtcactctcacctgtgcttccagcactggagcagtcaccagtggttactatccaaactggttccagcagaaacctggacaagcacccagggcactgatttatagtacaagcaacaaacactcctggacccctgcccggttctcaggctccctccttgggggcaaagctgccctgacactgtcaggtgtgcagcctgaggacgaggctgagtattactgcctgctcttctatggtggtgttcatgtcttcggagctgggaccaaggtcaccgtcctaggt

SEQ ID NO: 232 - DNA, 345 bp, synthetic construct
  source 1..345: mol_type = other DNA
gaggtgcagctggtggagtccgggggagacttagttcagccgggggggtccctaagactctcctgtgcagcctctggattcaccttcagtacctactggatgcactgggtccgccaagctcccgggcaggggctggtgtgggtctcacgtattaatggtaatgggagaatcacaaactacgcggactccgtgaagggccgattcaccgtctcccgagacaacgccaagaacacggtggatctgcaaatgaacagtctgagagccgaggacacggctgtgtattactgtgcaagaatcaatgaatggggagacgtctggggcaaagggaccacggtcaccgtctcg

SEQ ID NO: 233 - DNA, 339 bp, synthetic construct
  source 1..339: mol_type = other DNA
gatattgtgatgacccagaccccactctctctgtccgtcacccctggacagccggcctccatctcctgcaagtctagtcagagcctcctgcataatgatggaaagacctttttgcattggtacctgcagaagccaggccagtctccacagctcctaatctatgaagtttccagccgcttctctggagtgccagataggttcagtggcagcgggtcagggacagatttcacactgaaaatcagccgggtggaggctgaggatgttggagtttattactgcatgcaaggtatacaccttcctcccactttcggcggggggaccaaggtggaaatcaaacgt

SEQ ID NO: 234 - DNA, 351 bp, synthetic construct
  source 1..351: mol_type = other DNA
gaggtgcagctggtggagtccgggggaggcttagttcagccgggggggtccctgagactctcctgtgcagcctctggattcacattcagtagttactggatggaatgggtccgccaagctccagggaaggggctggtttgggtctcacatattagtagtgatgggagtgttacaaggtacgtggactccgtgaagggccgattcaccatctccagagacaacgccaagaacacgctgtatctgcaaatgaatagtctgagagccgaggacacgggtgtatattattgtgcaaaagatcttcactggaacgctcttgatgtgtggggccaagggacaatggtcaccgtctcg

SEQ ID NO: 235 - DNA, 327 bp, synthetic construct
  source 1..327: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaggtggaaatcaaacgt

SEQ ID NO: 236 - DNA, 363 bp, synthetic construct
  source 1..363: mol_type = other DNA
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagaggagtccgcagtggctggtacgctgatgcttttgatatctggggccaagggacaatggtcaccgtctcg

SEQ ID NO: 237 - DNA, 357 bp, synthetic construct
  source 1..357: mol_type = other DNA
gaggtgcagctggtggagtccgggggaggcttagttcagccgggggggtccctgggactctcctgtgcagcctctggattcaccttcagtaactactggatgcactgggtccgccaagctccagggaaggggctggtgtgggtctcgcgtattaatagtgatgggagtgacacaagctacgcggactccgtgaagggccgattcaccatctccagagacaacgccgagaacacgctgtatctgcacatgagcagtctgagagccgaggacacggctgtctattactgtgcaagggaattgggctacggtgactacggtatggacgtctggggcaaagggaccacggtcaccgtctcg

SEQ ID NO: 238 - DNA, 336 bp, synthetic construct
  source 1..336: mol_type = other DNA
cagtctgtgctgacgcagccaccctcagcgtctgggacccccgggcagagggtcaccatctcttgttctggaagcagctccaacatcggaagtcattctgtgatctggtaccagcagctcccaggaacggcccccaaactcctcgtctatagtgataatcagcggccctcaggggtccctgaccgattctctggctccaagtctggcacctcagcctccctggccatcagtgggctccagtctgaggatgaggctgattattactgtgcagcatgggatgacagcctgaatcatcttcatgtggtattcggcggagggaccaagctgaccgtccta

SEQ ID NO: 239 - DNA, 348 bp, synthetic construct
  source 1..348: mol_type = other DNA
gaggtgcagctggtggagtccgggggaggcttagttcagcctggggggtccctgagactctcctgtgcagcctccggattcatcttcagtaactactggatgcactgggttcgccaagctccaggggaggggctggtgtgggtctcacgtattaataaagatgggagtagcacattttacgcggactccgtgaagggccgattcaccatctccagagacaacgccaagaacacgctgtatctgcaaatgaacagtctgagagccgaggacacggctgtgtattattgtacaagagatttcgatttttggagtggctactggggccagggaaccctggtcaccgtctcg

SEQ ID NO: 240 - DNA, 330 bp, synthetic construct
  source 1..330: mol_type = other DNA
cagtctgccctgactcagcctgcctccgtgtctgggtctcctggacagtcgatcaccatctcctgcactggaaccagcagtgacgttggtggttgtgagtatgtctcctggtaccaacagcacccaggcaaagtccccaaactcatcatttatgaggtcagtaatcggccctcaggggtttctaatcgcttctctggctccaagtctggcaacacggcctccctgaccgtctctgggctccaggctgaggacgaggctgattatttctgcagctcctgtacaaccagcggctcttatgtcttcggagctgggaccaaggtcaccgtcgta

SEQ ID NO: 241 - DNA, 375 bp, synthetic construct
  source 1..375: mol_type = other DNA
gaggtgcagctggtggagtccgggggaggcgtagttcagcctggggagtccctgagactctcctgtgcagcctccggagtcaccttcagtagttactggatgcattgggtccgccaagttccagggaaggggctggtgtgggtcgcacgtattaatattgatgggaccagtacaacctacgcggactgtgtgaagggccgattcaccatctccagagacaacgccaagaacacgctgtatctgcaaatgagcagtctgagagccgaggacacggctgtttattactgtgcaacagagcgaggattacttagtggtggtcgctggcactcctcccactttgactactggggccagggaaccctggtcaccgtctcg

SEQ ID NO: 242 - DNA, 328 bp, synthetic construct
  source 1..328: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaaggtcctgatctataagacgtctagtttagaaagtggggtcccatcaaggttcagcggcagtggctctgggacagaattcactctcaccatcagcagcctgcagcctgatgattttgcaacttattactgccaacagtatgacagttcttctctcacgtggacgttcggccaagggaccaaggtggaaatcaaac

SEQ ID NO: 243 - DNA, 357 bp, synthetic construct
  source 1..357: mol_type = other DNA
gaggtgcagctggtggagtccgggggaggcttagttcagcctggggggtccctgagactctcctgtgcagcctctggattcaccttcagtaggttctggatgcactgggtccgccaggttccagggaaggggctggtgtggatctcacgtattaatagtgatgcgactagctcaagctacgcggactccgtgaagggccgattcaccatctccagagacaacgccaagaacacactgtttctgcaaatgaacagtctgagagccgaggacacggctgtctattactgtgcaaccagcaatacagctctggtttatttgcctgagaactggggccagggaaccctggtcaccgtctcg

SEQ ID NO: 244 - DNA, 322 bp, synthetic construct
  source 1..322: mol_type = other DNA
gaaattgtgttgacacagtctccagccaccctgtctttgtctccaggagagagagccaccctctcctgcagggccagtcagagtgttagcgactacttagcctggtaccaacagaaacctggccaggctcccaggctcctcatctatgatgcatccaacagggccactggcatcccagccaggttcagtggcggtgggtctgggacagacttcactctcaccatcagcagcctagagcctgaagattttgcagtttattactgtcagcagcgcagctactggcctccgacgttcggccaagggaccaaggtggaaatcaaac

SEQ ID NO: 245 - DNA, 334 bp, synthetic construct
  source 1..334: mol_type = other DNA
cagtctgccctgactcagcctccctccgcgtccgggtctcctggacagtcagtcaccatcacctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattatcactgcagctcatttgcaggcgacaacaattccccggtattcggcggagggaccaaattgaccgtcctag

SEQ ID NO: 246 - DNA, 359 bp, synthetic construct
  source 1..359: mol_type = other DNA
gaggtgcagctggtggagtccgggggaggcttaattcacccgggggggtccctgagactctcctgtgcagcctctggattcaccttcagtagctactggatgcactgggtccgccaagccccagggaaggggctggtgtgggtctcacatattaagagtgatgggagtaacacaatctacgcggactccgtgaagggccgattcaccatctccagagacaacgccaagaacacgctgtatctacaaatgaacagtctgagagccgaggacacggctgtgtattactgtggaagagggcgcagtttgaccccacgctcggccattgactactggggccagggaaccctggtcaccgtctc

SEQ ID NO: 247 - DNA, 347 bp, synthetic construct
  source 1..347: mol_type = other DNA
ctggtggagtctgggggaggcttggtccagcctggggggtccctgagactctcctgtgcagcctctggattcaccttcagcagttatgctatgcactgggtccgccaggctccagggaagggactggagtttgcttcagctattagtagtgatgggggtaccacatattacgcaaactctgtgaagggcagattcaccatctccagagacaattccaagaacacgctgtatcttcaaatgggcagcctgagggatgaagacatggctgtgtattactgtgcgagagatgatctgagcaccagctgggaccttgactactggggccagggaaccctggtcaccgtctc

SEQ ID NO: 248 - DNA, 331 bp, synthetic construct
  source 1..331: mol_type = other DNA
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgacgtcagtaatcggccctcaggggtttctaatcgcttctctggctccaagtctggcaacacggcctccctgaccatctctgggctccaggctgaggacgaggctgattattactgcagctcatatacaatcagcagcactaacgtcttcggaactgggaccaaggtcaccgtcctag

SEQ ID NO: 249 - DNA, 331 bp, synthetic construct
  source 1..331: mol_type = other DNA
cagtctgccctgactcagcctgcctccatgtctgggtctcctggacagtcgatcaccatctcctgcactggaaccagcagtgatgttggagctcataactttatctcctggtaccaacaacacccaggcaaagcccccaaactcatgatttttgaggtcaataagcggccctcaggggtttctaatcgcttctctggctccaagtctggcaacacggcctccctgacaatctctggcctccaggctgaggacgaggctgattatttctgctgctcatatgcaggttatagcacttgggtgttcggcggagggaccaagctgaccgtcctag

SEQ ID NO: 250 - DNA, 359 bp, synthetic construct
  source 1..359: mol_type = other DNA
gaggtgcagctggtggagtccgggggaggcgtagtccagcctggggggtccctgagactctcctgtgaagtctctggattcatcttcagtagctactggatgcactgggtccgccaagttgcagggaaggggctggtgtgggtctcacgtataaatagtgatgggagtagtacaagttacgcggactccgtgaagggccgattcaccatctccagagacaacgccaagaatacgctgaatctgcaaatgaattttctgagagccgaggacacggctgtgtattattgtgcaagagggggtgatcacggtgactacggcttctttgagtcttggggccagggaaccctggtcaccgtctc

SEQ ID NO: 251 - DNA, 353 bp, synthetic construct
  source 1..353: mol_type = other DNA
gaggtgcagctggtggagtccgggggagggttagttcagcctggggggtccctgagactctcctgtgcagcctctggattcaccttcagtaatcactggatgcactgggtccgccaagctccagggaaggggctgctgtgggtctcgcgtattgatactggtgggagtaccacaaactacgcggactccgtgaagggccgattcaccatctccagagacagcgccaagaacacggtgtatctacaaatgaacagtctgagagccgaagacacggctgtttattactgtgcaagaggcggtctcttctactacggtatggacgtctggggccaagggaccacggtcaccgtctc

SEQ ID NO: 252 - DNA, 328 bp, synthetic construct
  source 1..328: mol_type = other DNA
gaaattgtgttgacgcagtctccaggcaccctgtctttgtctccaggggaaagggccaccctctcctgcagggccagtcagactagtagcagcacctccttagcctggtatcagcagaaacgtggccaggctcccaggctcctcatctatggtgcatcccgcagggccactggcatcccagacaggttcagtggcagtgggtctgggacagacttcactctcaccatcagcagactggagcctgaagattttgcagtatattactgtcagcaatttagtggctcacctgcgtacacttttggccaggggaccaaggtggaaatcaaac

SEQ ID NO: 253 - DNA, 340 bp, synthetic construct
  source 1..340: mol_type = other DNA
gatattgtgatgacccagactccactctccctgcccgtcacccctggagagccggcctccatctcctgtaggtctagtcagagcctcttggatagtgctgatggaaacacctatttggactggtacctgcagaagccagggcagtctccacagctcctgatccatacgctttcccatcgggcctctggagtcccagacaggttcagtggcagtgggtcaggcactgatttcacactgaaaatcagcagggtggaggctgaagatgttggagtttattactgcatgcaacgtatagcctttcccgtcactttcggccctgggaccaaagtggatatcaaac

SEQ ID NO: 254 - DNA, 347 bp, synthetic construct
  source 1..347: mol_type = other DNA
gaggtgcagctggtggagtccgggggaggcttagttcagcctggggggtccctgagactctcctgtgcagcctctggattcaccttcagtaactactggatgcactgggtccgccaagctccaggggaggggctggagtgggtctcacgtattaatagtgatgggaataaggtaagttacgcggactccgtgaggggccgattcaccatctccagagacaacgccaagaacacgctgtatctacaaatgaacagcctgacaggcgaggacacggctgtgtattattgtgcaagatctaactggggatcggcagactactggggccagggaaccctggtcaccgtctc

SEQ ID NO: 255 - DNA, 377 bp, synthetic construct
  source 1..377: mol_type = other DNA
gaggtgcagctggtggagtctgggggaggctcagttcagcctggggggtccctgagactctcctgtgctgcctctggattcaccttcagtagatactggatgcactgggtccgccaggctccaggggaggggctcgtttgggtctcacgtataaatagtgatgagactgacaagctttacgcggactccgtgaagggccgattctccatcttcagagacaacgccaagaacacactatatctgcaaatgaacagactgagagccgaggacacggctgtatactactgtgcaagagatcgagaggatgttgtagtggggccagctactcaacacaccatctttaactcctggggccagggaaccctggtcaccgtctc

SEQ ID NO: 256 - DNA, 334 bp, synthetic construct
  source 1..334: mol_type = other DNA
cagtctgccctgactcagcctgcctccgtgtctgggtctcctggacagtcgatcaccatctcctgcactggaaccagcagtgatgttgggagttctaactttgtctcctggtaccaacaacacccaggcaaagcccccaaactcatcctttatgaggtcagtaagcggcccgctggagtttctagtcgcttctctggctccaggtctggcaacacggcctccctgacaatctctggagcccaggctgaggacgaggctgactattcctgctgctcaacttcttctgttggcactctttatgtcttcggaactgggaccaaggtaaccgtcctag

SEQ ID NO: 257 - DNA, 331 bp, synthetic construct
  source 1..331: mol_type = other DNA
cagtctgtgctgacgcagccgccctcagtgtctgcggccccaggacagaaggtcaccatctcctgctctggaagcaactacaacattgggaataattatgtatcctggtaccagcgactcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagggctggggtgttcggcggagggaccaagctgaccgtcctag

SEQ ID NO: 258 - DNA, 356 bp, synthetic construct
  source 1..356: mol_type = other DNA
gaggtgcagctggtggagtccgggggaggcttagttcagcctggggggtccctgagactctcctgtgcagcctctggattcactttcagtaggcactggatgcactgggtccgccaagctccagggaaggggctggtgtgggtctcacgtattaatagtgatgggagtagcacaagtaacgcggactccgtgaagggccgaatcaccatctccagagacaacggcaagaacacgctgtatctgcagatgaacagtctgagagccgaggacacggctgtgtattactgtgcaagggagatagcatcggggacagatgcttttgatatctggggccaagggacaatggtcaccgtctc

SEQ ID NO: 259 - DNA, 368 bp, synthetic construct
  source 1..368: mol_type = other DNA
gaggtgcagctggtggagtctgggggaggcttagtaaagcctggggagtcccttagactctcctgtgtaggttctggattcactctcactaacgcctggatgatctgggtccgccagacttcagggaaggggctggaatgggttggtcgcatcaagagtaaaattgatggtggggcaatcgactacggtgcacccgtgaaaggtagatttaccatctcaagagatgatacaaaaaacacggtgtatctgcaaatgaacagcctgcaaaccgacgacacaggcgtctatttctgtaccacagatcgttatagtactggctactacggcatggacgactactggggccagggaaccctggtcaccgtctc

SEQ ID NO: 260 - DNA, 317 bp, synthetic construct
  source 1..317: mol_type = other DNA
tcctatgagctgactcagccaccctcagtgtccgtgtccccaggacagacagccaccatcacctgctctggagataaattgggagataaatattctttctggtatcaacagaagccaggccagtcccctgtggtggtcatctatcaagattccaagcggccctcagggatccctgagcgattctctggctccaactctgggaacacagccactctgaccatcagcgggacccaggctatggatgaggctgactattactgtcaggtgtgggacaccgactctgcagtcttcggaactgggaccagggtcaccgtcct

SEQ ID NO: 261 - DNA, 322 bp, synthetic construct
  source 1..322: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaaggtggaaatcaaac

SEQ ID NO: 262 - DNA, 341 bp, synthetic construct
  source 1..341: mol_type = other DNA
gaggtgcagctggtggagtctgggggaggcctggtcaagcctggggggtccctgagactctcctgtgcggcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatacgatttctcgatactggggccagggaaccctggtcaccgtctc

SEQ ID NO: 263 - DNA, 368 bp, synthetic construct
  source 1..368: mol_type = other DNA
gaggtgcagctggtggagtctgggggaggcctggtcaagcctggggggtccctgagactttcctgtgcagcctctggattcaccttcagtagttacagcatgaactgggtccgccaggctccagggaaggggctggagtgggtctcatccattagtggtagtagtagttacatgcactacgcagagtcagtgaagggccgattcaccatctccagagacaacgccaagaactcactgtatctgcaaatgaatagcctgagagccgaggacacggctgtgtattactgtgcgagtacggggattatcacgtattactatgcttcgggggtccctgactactggggccagggaaccctggtcaccgtctc

SEQ ID NO: 264 - DNA, 331 bp, synthetic construct
  source 1..331: mol_type = other DNA
cagtctgtgctgactcagccaccctcagcgtctgggacccccgggcagagggtcaccatgtcttgttctggaagcagctccaacatcggaagaaattatgtatactggtaccagcagctcccaggaacggcccccaaactcctcatctataggaataatcagcgaccctcaggggtccctgaccgattctctggctccaagtctggcacctcagtctccctggccatcagtgggctccggtccgaggatgaggctgattattactgtgcagcatgggatgacagcctgagtggtgtggtattcggcggagggaccaagctgaccgtcctag

SEQ ID NO: 265 - DNA, 322 bp, synthetic construct
  source 1..322: mol_type = other DNA
gaaattgtgttgacgcagtctccaggcaccctgtctttgtctccaggggaaagagccaccctctcctgcagggccagtcagagtgttagcagcaacttagcctggtaccagcagaaacctggccaggctcccagggtcctcatctatggtgcatccagcagggccactggcatcccagacaggttcagtggcagtcggtctgggacagacttcactctcaccatcagcagactggagcctgaagattttgcagtgtattactgtcagcggtatgataactcacctcggacgttcggccaagggaccaaggtggaaatcaaac

SEQ ID NO: 266 - DNA, 359 bp, synthetic construct
  source 1..359: mol_type = other DNA
gaggtgcagctggtggagtctgggggaggcttggtaaagcctggggggtcccttagactctcctgtgcagcctctggtttcactttcattaacgcctggatgaactgggtccgccaggctccagggaaggggctggagtgggtcggcctaattaaaagcaaaactgatggtgggacaatagactacgctgcacccgtgaaaggcagattcactatttcaagagatgattcagaaaaaatgttgtatctgcaaatggacagcctgaagaccgaggacacagccgtgtattactgtaccacaatgtatggctggaaggacgcgagggactattggggccagggaaccctggtcaccgtctc

SEQ ID NO: 267 - DNA, 325 bp, synthetic construct
  source 1..325: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggttcctcatctatggtgcgtccagtagggccactggcatcccggacaggttcagtggcagtgggtctgggacagacttcactctcaccatcagcagactggagcctgaagattttgcagtgtattactgtcagcagtatggtagctcaccgtacacttttggccaggggaccaaggtggaaatcaaac

SEQ ID NO: 268 - DNA, 365 bp, synthetic construct
  source 1..365: mol_type = other DNA
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaaaagtgatgggagtagcacaagctacgcggactccgtgaagggccgattcaccatctccagagacaacgccaagaacacgctgtatctgcaaatgaacagtctgagagccgaggacacggctgtgtattactgtgcaagagggggtggcagcagcaactggtacccgggtttctttgactactggggccagggaaccctggtcaccgtctc

SEQ ID NO: 269 - DNA, 356 bp, synthetic construct
  source 1..356: mol_type = other DNA
gaggtgcagctgttggagtctgggggaggcttggtacagccgggggggtccctgagactctcctgtgtagcctctggattcaccttcagcagctatgccatgagctgggtccgccaggctccagggaaggggctggagtgggtctctagtattagtgctagtggtggtagcacatattacgcagactccgtgaagggccggttcaccatctccagagacaattccaagaacacgctgtatctgcaaatgaacagcctgagagccgaggacacggccgtatattactgtgggaattggccggaaggattcccggcctactttcactactggggccagggaaccctggtcaccgtctc

SEQ ID NO: 270 - DNA, 322 bp, synthetic construct
  source 1..322: mol_type = other DNA
gaaatagtgatgacgcagtctccagccaccctgtctgtgtctccaggggaaagagtcaccctctcctgcagggccagtcagagtgttaccagcgagttggcctggtaccagcagaaacctggccaggctcccaggctcctcatctatgatgcatccaccggggccactggtatcccagccaggttcagtggcagtgggtctgggacagacttcactctcaccatcagcagcctgcagtctgaagattttgcagtttattactgtcagcagcataataactggcctctcactttcggcggcgggaccaaggtggaaatcaaac

SEQ ID NO: 271 - DNA, 368 bp, synthetic construct
  source 1..368: mol_type = other DNA
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaagagtgatgggaggagcataagttacgcggactccgtgaagggccgattcaccatctccagagacaacgccaagaacacgctgtatctgcaaatgaacagtctgagagccgaggacacggctgtgtattactgtgcaagagatccccacggcacagcagctcctccccgtgatgcttttgatatctggggccaagggacaatggtcaccgtctc

SEQ ID NO: 272 - DNA, 331 bp, synthetic construct
  source 1..331: mol_type = other DNA
cagtctgccctgactcagcctgcctccgtgtctgggtctcctggacagtcgatcaccatctcctgcactggaaccagcggtgatgttgggagttataaccttgtctcctggtaccaacagtacccaggcaaagcccccaaactcatgatttatgaggtcgataagcggccctcaggggtctctaatcgcttctctggctccaagtctggcaacacggcctccctgacaatttctgggctccgggctgaggacgaggctcattatcactgcttctcatatgcaggtagtttgactttggtattcggtggagggaccaagttgaccgtcctag

SEQ ID NO: 273 - DNA, 331 bp, synthetic construct
  source 1..331: mol_type = other DNA
cagtctgtgctgactcagccaccctcagtgtctgggacccccgggcagagggtcaccatctcttgttctggaagcagctccaacatcggaagtaaaactgcaaactggtaccagaagctcccaggaacggcccccaaactcctcatctatagtaacaatcagcggccctcaggggtccctgaccgattctctggctccaagtcaggcacctcagcctccctggctatcagtgggctccagtctgaggatgaggctgggtactactgcacagcatgggatgacagcctgaatggtccggtgttcggcggagggaccaagctgaccgtcctag

SEQ ID NO: 274 - DNA, 347 bp, synthetic construct
  source 1..347: mol_type = other DNA
gaggtgcagctggtggagtctgggggaggcttggtccagcctggggggtccctgagactctcctgtggagcctctggattcaccgtcagtggcaagtatatgacctgggcccgccaggctcctgagaagggactggagtgggtctcagctatctatcgcggtggtggcacatactacgcagactccgtgaagggcagattcaccatctccagagacaattccaaaaacatgttatatcttcaaatgaacagcctgagagccgaggacacggctgtgtattactgtggggggtccgtgatggtgagtgctactgactactggggccagggaaccctggtcaccgtctc

SEQ ID NO: 275 - DNA, 322 bp, synthetic construct
  source 1..322: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaaggtggagatcaaac

SEQ ID NO: 276 - DNA, 341 bp, synthetic construct
  source 1..341: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatcaaattagtggttactggggccagggaaccctggtcaccgtctc

SEQ ID NO: 277 - DNA, 347 bp, synthetic construct
  source 1..347: mol_type = other DNA
gaggtgcagctggtggagtccgggggaggcttagttcagcctggggggtccctgagactctcctgtgtagcctctggattcaccttcagtagctactggatgcactgggtccgccaagctccagggaaggggctggtgtgggtctcacggatttatcctgatgggactactactgcaaactacgcggactccgtgaagggccgattcaccatctccagagacaacgccaagaacacggtgtatctgcaaatgaacagtctgagagccgaggacacggctgtttattactgtgcaagagatcttcgggagtctgattactggggccagggaaccctggtcaccgtctc

SEQ ID NO: 278 - DNA, 331 bp, synthetic construct
  source 1..331: mol_type = other DNA
cagtctgccctgactcagcctgcctccgtgtctgggtctcctggacagtcgatcaccatctcctgcactggaaccagcagtgacgtgggtaattataattatgtctcctggcaccaacaacacccagggaaagcccccaaactcatgatttatgatgtcagtaatcggccctcaggggtttctaatcgcttctctggctccaagtctggcaacacggcctccctgaccatctctgggctccaggctgaggacgaggctgattattactgcaactcatatacaacctacagcactcacgtcttcggaactgggaccaaggtcaccgtcctag

SEQ ID NO: 279 - protein, 21 aa, synthetic construct
KPAVIPDREALYQDIDEMEEC

SEQ ID NO: 280 - protein, 21 aa, synthetic construct
KPAVIPDREMLIQSIVEMEEC

SEQ ID NO: 281 - protein, 20 aa, synthetic construct
PAVIPDRPMLFQSIVEMEEC

SEQ ID NO: 282 - protein, 9 aa, synthetic construct
  SITE 5: note = X= V or T
PMLFXQSIV

SEQ ID NO: 283 - protein, 8 aa, synthetic construct
PMLFQSIV

SEQ ID NO: 284 - DNA, 352 bp, synthetic construct
  source 1..352: mol_type = other DNA
gaggtgcagctggtggagtctgggggaggcgtggcccagcctgggacgtctctgagactctcctgtgaagcgtctggattcaccttccgtgactatgccatgcgctgggtccgccaggctccaggcaaggggctggagtgggtggcatttatatggaatgatggaagtaagaaatattacacagactccgtgaggggccgattcaccatctccagagacaattccaagaacatgctgtatctgcaaatggacagcctgagagccgaggacacggctctttattactgtgccagaaagatgagtgaagatgatgcttttgatctgtggggccaagggacaatggtcaccgtct

SEQ ID NO: 285 - DNA, 357 bp, synthetic construct
  source 1..357: mol_type = other DNA
gaggtgcagctggtggagtccgggggaggcttagttcagccgggggggtccctgagactctcctgtgcagcctctggattcacattcagtagttactggatggaatgggtccgccaagctccagggaaggggctggtttgggtctcacataaagatattagtagtgatgggagtgttacaaggtacgtggactccgtgaagggccgattcaccatctccagagacaacgccaagaacacgctgtatctgcaaatgaatagtctgagagccgaggacacgggtgtatattattgtgcaaaagatcttcactggaacgctcttgatgtgtggggccaagggacaatggtcaccgtctcg

SEQ ID NO: 286 - protein, 110 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTLLFGGGTKLTVL

SEQ ID NO: 287 - protein, 10 aa, synthetic construct
CSYAGSYTLL

SEQ ID NO: 288 - DNA, 331 bp, synthetic construct
  source 1..331: mol_type = other DNA
cagtctgccctgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcagtaagcggccctcaggggtccctgatcgcttctctggctccaagtctggcaacacggcctccctgaccatctctgggctccaggctgaggatgaggctgattattactgctgctcatatgcaggcagctacactttgttattcggcggagggaccaagctgaccgtcctag

SEQ ID NO: 289 - protein, 121 aa, synthetic construct
EVQLVESGGGLVQPGGSLRLSCAASGFTFSSYWMYWVRQAPGKGLVWVSRINSDGSTTNYADSVKGRFTISRDNAKNTLYLQMNSLRAEDTAVYFCARYAHLGIGWYFDLWGRGTLVTVSS

SEQ ID NO: 290 - protein, 8 aa, synthetic construct
INSDGSTT

SEQ ID NO: 291 - protein, 14 aa, synthetic construct
ARYAHLGIGWYFDL

SEQ ID NO: 292 - DNA, 364 bp, synthetic construct
  source 1..364: mol_type = other DNA
gaggtgcagctggtggagtccgggggaggcttagttcagcctggggggtccctgagactctcctgtgcagcctctggattcaccttcagtagctactggatgtactgggtccgccaagctccagggaaggggctggtgtgggtctcacgtattaatagtgatgggagtaccacgaactacgcggactccgtgaagggccgattcaccatctccagagacaacgccaagaacacgctgtatctgcaaatgaacagtctgagagccgaggacacggctgtgtatttctgtgcaagatatgcccacctggggataggttggtacttcgatctctggggccgtggcaccctggtcaccgtctcctcag

SEQ ID NO: 293 - protein, 108 aa, synthetic construct
DIQMTQSPSTLSASAGDRVTITCRASQSVSKYLAWYQQKPGKAPRLLIYKASSLQSGVPSRFSGSGSGTEFTLTISSLQPDDFATYYCQHYNTYPSWTFGQGTKVEIK

SEQ ID NO: 294 - protein, 6 aa, synthetic construct
QSVSKY

SEQ ID NO: 295 - skipped

SEQ ID NO: 296 - protein, 10 aa, synthetic construct
QHYNTYPSWT

SEQ ID NO: 297 - DNA, 324 bp, synthetic construct
  source 1..324: mol_type = other DNA
gacatccagatgacccagtctccttccaccctgtctgcatctgcaggagacagagtcaccatcacttgccgggccagtcagagtgttagtaagtacttggcctggtatcagcagaaaccagggaaagcccctaggctcctgatctataaggcatctagtttacaaagtggggtcccatcaaggttcagtggcagtggatctgggacagaattcactctcaccatcagcagcctgcagcctgatgattttgctacttattactgccaacactataatacttatccttcgtggacgttcggccaagggaccaaggtggaaatcaaa

SEQ ID NO: 298 - protein, 117 aa, synthetic construct
EVQLVESGGGVAQPGTSLRLSCEASGFTFRDYAMRWVRQAPGKGLEWVAFIWNDGSKKYYTDSVRGRFTISRDNSKNMLYLQMDSLRAEDTALYYCARKMSEDDAFDLWGQGTMVTV

SEQ ID NO: 299 - protein, 8 aa, synthetic construct
GFTFRDYA

SEQ ID NO: 300 - protein, 8 aa, synthetic construct
IWNDGSKK

SEQ ID NO: 301 - protein, 12 aa, synthetic construct
ARKMSEDDAFDL

SEQ ID NO: 302 - protein, 107 aa, synthetic construct
DIQMTQSPSSLSASVGDRVTITCRASQGIRNDLGWYQQKPGKAPKLLIYGASSLQSGVPSRFSGSGSGTDFTLTISSLQPEDIATYYCLQEYTYPLTFGGGTKVEIK

SEQ ID NO: 303 - protein, 6 aa, synthetic construct
QGIRND

SEQ ID NO: 304 - protein, 9 aa, synthetic construct
LQEYTYPLT

SEQ ID NO: 305 - DNA, 321 bp, synthetic construct
  source 1..321: mol_type = other DNA
gacatccagatgacccagtctccatcctccctgtctgcatccgtaggagacagagtcaccatcacttgccgggcaagtcagggcattagaaatgatttaggctggtatcagcagaaaccagggaaagcccctaagctcctgatctatggtgcatccagtttacaaagtggggtcccatcaagattcagcggcagtggatctggcacagatttcactctcaccatcagcagcctgcagcctgaagatattgcaacctattactgtctacaagaatacacttacccgctcactttcggcggagggaccaaggtggaaatcaaa

SEQ ID NO: 306 - protein, 119 aa, synthetic construct
EVQLVESGGGLVQPGGSLRLSCAASGFTFSDYYMDWVRQAPGKGLEWVGRTRNKANSYTTEYAASVKGRFTISRDDSKNSLYLQMNSLKTEDTAVYYCARFLLVADAFDIWGQGTMVTV

SEQ ID NO: 307 - protein, 8 aa, synthetic construct
GFTFSDYY

SEQ ID NO: 308 - protein, 10 aa, synthetic construct
TRNKANSYTT

SEQ ID NO: 309 - protein, 12 aa, synthetic construct
ARFLLVADAFDI

SEQ ID NO: 310 - DNA, 358 bp, synthetic construct
  source 1..358: mol_type = other DNA
gaggtgcagctggtggagtctgggggaggcttggtccagcctggagggtccctgagactctcctgtgcagcctctggattcaccttcagtgactactacatggactgggtccgccaggctccagggaaggggctggagtgggttggccgcactagaaacaaagctaacagttacaccacagaatacgccgcgtctgtgaaaggcagattcaccatctcaagagatgattcaaagaactcactgtatctgcaaatgaacagcctgaaaaccgaggacacggccgtgtattactgtgctagatttctgctggttgcggatgcttttgatatctggggccaagggacaatggtcaccgtct

SEQ ID NO: 311 - protein, 77 aa, synthetic construct
AGKPAVIPDREALYQDIDEMEECLDEAGKPAVIPDREALYQDIDEMEECLDEAGKPAVIPDREALYQDIDEMEECLD

SEQ ID NO: 312 - protein, 21 aa, synthetic construct
IYPLEDMAEPKVERIDAQEDC

SEQ ID NO: 313 - protein, 109 aa, synthetic construct
EIVLTQSPGTLSLSPGERATLSCRASQTSSSTSLAWYQQKRGQAPRLLIYGASRRATGIPDRFSGSGSGTDFTLTISRLEPEDFAVYYCQQFSGSPAYTFGQGTKVEIK

(19) United States
(12) Patent Application Publication
(10) Pub. No.: US 2025/0172552 A1
(43) Pub. Date: May 29, 2025

(54) MONOCLONAL ANTIBODIES FOR DETECTING KAWASAKI DISEASE ANTIGENS

(71) Applicants: Ann and Robert H. Lurie Children's Hospital of Chicago, Chicago, IL (US); Loyola University of Chicago, Maywood, IL (US)

(72) Inventors: Anne H. Rowley, Chicago, IL (US); Stanford T. Shulman, Chicago, IL (US); Susan C. Baker, Maywood, IL (US)

(21) Appl. No.: 18/842,643

(22) PCT Filed: Mar. 3, 2023
(86) PCT No.: PCT/US2023/063740

§ 371 Date: Aug. 29, 2024

Related U.S. Application Data
(60) Provisional application No. 63/268,847, filed on Mar. 3, 2022.
(60) Provisional application No. 63/476,373, filed on Dec. 20, 2022.

(51) Int. Cl. G01N 33/543; C07K 7/08; C07K 16/18; G01N 33/68

(52) U.S. Cl. CPC G01N 33/54388; C07K 7/08; C07K 16/18; G01N 33/6854; C07K 2317/24; C07K 2317/52; C07K 2317/565; G01N 2333/46; G01N 2800/328

(57) ABSTRACT

The present disclosure provides monoclonal antibodies that target intracytoplasmic inclusion bodies in KD and methods for their use. Wherein an isolated antibody or antigen binding fragment thereof comprises: a heavy chain variable domain comprising: CDRH1, CDRH and CDRH3 sequences; and a light chain variable domain comprising: CDRL1, CDRL2, and CDRL3 sequences.

### CROSS-REFERENCE TO RELATED APPLICATIONS

This application claims the benefit of and priority to U.S. Provisional Application No. 63/268,847 filed Mar. 3, 2022, and U.S. Provisional Application No. 63/476,373, filed Dec. 20, 2022, each of which is incorporated herein by reference in its entirety.

### STATEMENT REGARDING FEDERALLY SPONSORED RESEARCH

This invention was made with government support under grant 5RO1AI150719 awarded by the National Institutes of Health. The government has certain rights in this invention.

### REFERENCE TO AN ELECTRONIC SEQUENCE LISTING

The contents of the electronic sequence listing (702581.02308.xml; Size: 289,280 bytes; and Date of Creation: Mar. 3, 2023) is herein incorporated by reference in its entirety.

### BACKGROUND

Kawasaki disease (KD) is an acute febrile illness of childhood with the clinical features of oral and conjunctival erythema, rash, cervical adenopathy, and redness and swelling of the hands and feet. The disorder is distinctive in its potential to cause severe and persisting coronary artery aneurysms that can lead to myocardial infarction, aneurysm rupture, and sudden death^{1,2,3} Many other infectious and inflammatory conditions of childhood share clinical features with KD, and distinguishing KD from these conditions is important to allow for timely treatment with intravenous gammaglobulin, which reduces the prevalence of coronary artery aneurysms. However, diagnosis is currently difficult, because the specific etiology has not been determined and there is no diagnostic test. This recently led to confusion between KD and Multisystem Inflammatory Syndrome in Children (MIS-C), which share some clinical features⁴.

Controversy exists as to the most likely etiologic agent(s) for KD, with some investigators favoring diverse infectious agents as triggers and others favoring a single agent or closely related group of agents as the cause^{5,6}. Close examination of clinical and epidemiologic features provides clues about the most likely etiology. Most cases of KD occur in children 6 months to 5 years of age, a typical age range for acquisition of a ubiquitous infectious agent. Epidemics and outbreaks are commonly reported throughout the world, typical of infection with a single pathogen. The illness rarely recurs, arguing against both a hypothesis of multiple inciting pathogens and of autoimmune disease. Asian children experience the highest attack rates of KD, although children of all racial and ethnic groups can develop KD. In combination, these features strongly support infection with a presently unidentified ubiquitous infectious agent that usually results in asymptomatic infection, but causes KD in genetically susceptible children. Applicant's prior studies support a novel respiratory virus that forms intracytoplasmic inclusion bodies in infected tissues as the cause⁷⁻¹⁰. Recently, mitigation strategies such as masking and social distancing put into place during the COVID-19 pandemic resulted in a significant reduction in KD cases worldwide, providing a real-world experiment supporting a presently unknown respiratory virus as the most likely causative agent^{11-15.}

The applicants previously reported an oligoclonal IgA response in acute KD coronary arteries¹⁶, supporting a specific immune response to a causative agent. Wang and colleagues performed scRNA seq and identified an oligoclonal B cell response in peripheral blood mononuclear cells from children with KD, consistent with the response in the coronary artery tissues⁶. A protein epitope targeted by monoclonal antibodies (MAbs) derived from clonally expanded peripheral blood plasmablasts from children with recently diagnosed KD was also recently identified¹⁷. Incubation of KD MAbs with human protein arrays that display full-length or near full-length proteins of 80% of the canonical proteome did not yield a human target of the antibodies, and a microbial, likely viral, target seems most likely, especially in view of the binding of the MAbs to intracytoplasmic inclusion bodies in KD tissues that the inventors observed to have virus-like particles in close proximity^{10, 17}. Although a recent study proposed that KD pathogenesis was linked to an IL15/IL15RA cytokine storm⁵, prior transcriptional profiling study of KD coronary artery tissues showed no difference in expression of IL15 and IL15RA in coronary artery tissues of children with KD compared with childhood controls¹⁸. Instead, the KD coronary artery immune signature revealed activated cytotoxic T lymphocyte and type I interferon-induced gene upregulation, features characteristic of antiviral response¹⁸.

### SUMMARY OF THE DISCLOSURE

The present disclosure provides antibodies and methods for detecting intracytoplasmic inclusion bodies.

In an aspect, provided herein is an isolated intracytoplasmic inclusion body (ICI) antibody or antigen binding fragment thereof comprising: 1. An isolated antibody or antigen binding fragment thereof comprising:
a heavy chain variable domain comprising:
a CDRH1 region selected from the group consisting of SEQ ID NOs: 2, 14, 22, 30, 42, 50, 62, 78, 86, 93, 101, 123, 126, 139, 149, 152, 164, 176, 186, and 205;
a CDRH2 region selected from the group consisting of SEQ ID NOs: 74, 15, 23, 31, 35, 43, 51, 63, 70, 75, 79, 87, 94, 102, 111, 114, 127, 136, 140, 153, 165, 168, 177, 183, 187, 193, 206, and 212;
a CDRH3 region selected from the group consisting of SEQ ID NOs: 4, 16, 14, 32, 36, 44, 52, 64, 71, 76, 80, 88, 95, 103, 112, 115, 124, 128, 137, 141, 150, 154, 166, 169, 178, 184, 188, 194, 207, 210, and 213; and
a light chain variable domain comprising:
a CDRL1 region selected from the group consisting of SEQ ID NOs: 6, 10, 18, 26, 37, 46, 54, 58, 66, 38, 82, 97, 105, 46, 119, 129, 132, 143, 146, 156, 160, 171, 180, 190, 197, 201, and 215;
a CDRL2 region selected from the group consisting of SEQ ID NOs: 7, 11, 27, 28, 47, 55, 59, 67, 39, 83, 98, 106, 120, 133, 157, 161, 198, 202, and 208; and
a CDRL3 region selected from the group consisting of SEQ ID NOs: 8, 12, 20, 28, 39, 48, 56, 60, 68, 73, 84, 99, 107, 109, 117, 121, 130, 134, 144, 147, 158, 162, 172, 174, 181, 191, 199, 203, and 216.

In another aspect, provided herein is a method of diagnosing Kawasaki Disease in a subject, comprising the steps of: i) obtaining a sample from a subject suspected of having Kawasaki Disease; ii) contacting the sample with any of the antibodies or antigen binding fragments disclosed herein; and ii) detecting the binding of the antibody in the sample, wherein binding of the antibody indicates the presence of Kawasaki Disease. In an embodiment, the sample is a blood sample. In an embodiment, detecting the binding of the antibody in the sample is carried out using ELISA, Western blot, immunostaining, immunoprecipitation, flow cytometry, sensor chips, or magnetic beads.

In another aspect, provided herein is a method of detecting intracytoplasmic inclusion bodies in a subject, comprising the steps of: i) obtaining a sample from a subject suspected of having Kawasaki Disease; ii) contacting the sample with any of the antibodies or antigen binding fragments; and ii) detecting the binding of the antibody in the sample, wherein binding of the antibody indicates the presence of intracytoplasmic inclusion bodies. In an embodiment, the sample is a blood sample.

In an aspect, provided herein is a peptide comprising SEQ ID NO: 281 or a peptide comprising a sequence with 95% similarity to SEQ ID NO: 281.

In an aspect, provided herein is a method of detecting associated with Kawasaki disease in a subject comprising the steps of:
i) obtaining a sample comprising antibodies from a subject suspected of having Kawasaki disease;
ii) contacting the sample with any of the peptides disclosed herein; and
iii) detecting the binding of antibodies to the peptide to form a peptide-antibody complex, wherein the presence of a peptide-antibody complex confirms the presence of antibodies associated with Kawasaki disease in the subject.

In another aspect, provided herein is a kit comprising i) any of the antibodies or antigen binding fragments thereof, or peptides provided herein; and ii) a detection reagent.

### BRIEF DESCRIPTION OF DRAWINGS

FIGS. 1A-1B. Approach to identify antigen-antibody response in KD patients. A. Scheme for analysis of plasmablast response in patients with KD. B. Diagram of the two approaches used to identify a consensus KD epitope and the convergent antibody response in plasmablasts from patients with KD.

FIG. 2A-2F. Amino acid substitution scans using MAbs from multiple KD patients support a consensus PMLFQSIV (SEQ ID NO: 315) epitope. The base peptide used is shown on the far left of each scan. Each amino acid of the base peptide is replaced by each of 20 amino acids and the relative binding of the peptide with each amino acid replacement is shown. Red indicates strong binding while blue indicates inhibition of binding relative to the base peptide. Scans using MAbs from KD patients 1, 3, 5, 6, 7, and 10 are shown; additional scans performed using MAbs from KD patients 2, 4, and 8 gave similar results.

FIGS. 3A-3B. KD MAbs show preferential binding to KD peptide 3 over KD peptide 1. A. ELISA of a subset MAbs to the three peptides. For all antibodies recognizing KD peptide 3, binding to KD peptide 3 was significantly greater than KD peptide 1 with p<0.01. B. Western blots of KD peptide 3 (FP3) and KD peptide 1 (FP1) mouse Fc fusion proteins probed with KD MAbs KD4-2H4 and KD6-2H3. Human IgG is used as a positive control for the secondary anti-human IgG antibody, and stripped blots were re-probed with anti-mouse IgG to assess protein loading. KD peptide 3 is SEQ ID NO: 281, KD peptide 2 is SEQ ID NO: 280, KD peptide 1 is SEQ ID NO: 279.

FIGS. 4A-4B. Analysis of CDR3 sequences of KD MAbs supports a convergent antibody response in KD patients. A. Alignment of CDR3 amino acid sequences from KD MAbs that recognize KDpeptide 3. B. Motif-based sequence analysis of CDR3 amino acid sequences of KD MAbs binding to KD peptide 3. Analysis was performed using the MEME suite (https://meme-suite.org/meme/), revealing a statistically significant consensus motif at 2.3e-6.

FIG. 5. KD MAbs show preferential binding to KD peptide 3 over KD peptide 1. KD peptide 3 is SEQ ID NO: 281, KD peptide 2 is SEQ ID NO: 280, KD peptide 1 is SEQ ID NO: 279.

FIG. 6. Serial dilutions of MAb reactivity against KDpeptide3

FIG. 7. Schematic depicting a model of KD pathogenesis subsequent to infection of the subject with a ubiquitous viral pathogen which may cause mild or asymptomatic infection in other subjects.

### DETAILED DESCRIPTION OF THE INVENTION

The present disclosure describes monoclonal antibodies and antigen binding fragments thereof that can bind to intracytoplasmic inclusion bodies (ICI) and that are specific for antigens found in subjects with Kawasaki Disease (KD) The present disclosure also describes methods of using the disclosed monoclonal antibodies to diagnose and treat KD in a subject.

The present disclosure also provides a peptide specific for the detection of antibodies associated with Kawasaki disease, and the use of this peptide in assays and methods for detection of KD in children. One of these peptides (KD3) binds significantly more strongly to the majority of the Kawasaki disease monoclonal antibodies than previously reported peptides. Thus, protein constructs that use this peptide (KD3) sequence (or epitope) may perform even better than the previously reported peptides in serologic assays for Kawasaki disease, which is urgently needed.

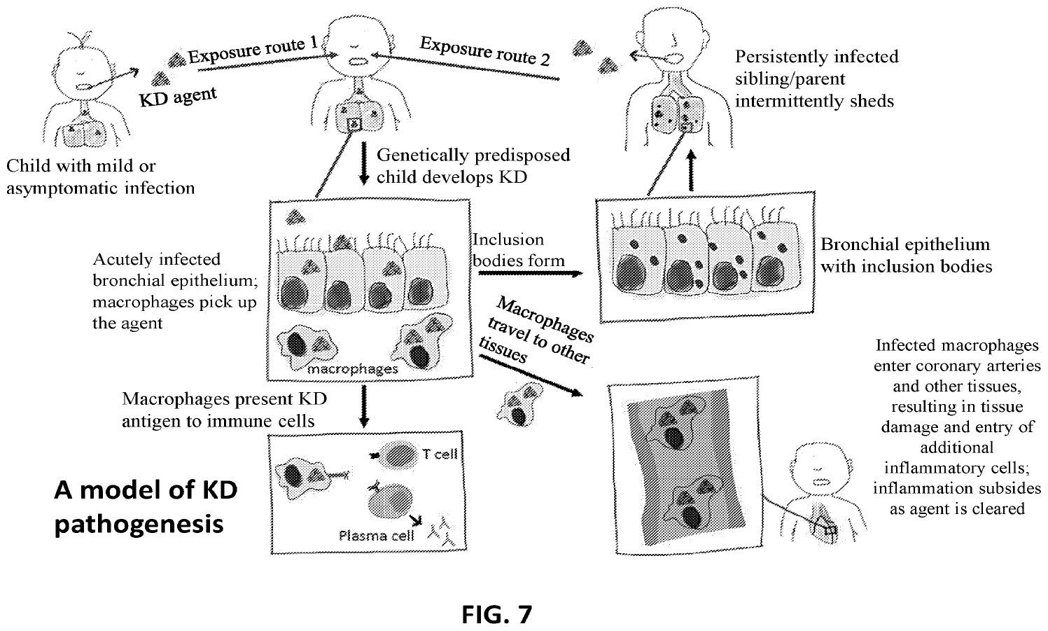

The present disclosure also provides analysis of antibodies and the convergent VH3-74 antibody response in children with Kawasaki Disease recognizing this epitope (see FIG. 7). Of the original group of 11 children with Kawasaki disease in whom the inventors cloned the acute plasmablast response, 9 were identified as having an antibody binding to this specific protein epitope. The inventors presently have identified a total of 16 antibodies from the 9 children that recognize this epitope. The identification of a convergent VH3-74 antibody response in children with Kawasaki disease to a single non-human protein antigen strongly indicates a single infectious etiologic agent for the disease, and the identified epitope appears to represent a prominent epitope or mimotope of this agent. Thus, these antibodies may further provide help for not only treatment of KD and screening mechanisms for detecting KD, but also for determining the causality of KD.

### Antibodies and Antigen Binding Domains Specific for Kawasaki Disease

The inventors herein disclose novel antibodies and antigen binding fragments thereof that are specific for antigens found in Kawasaki disease. Kawasaki Disease (KD) is a febrile illness of young childhood that has clinical and epidemiologic features of an infectious disease including epidemics with geographic wavelike spread. In some cases, Kawasaki disease manifests only as prolonged fever, making timely diagnosis difficult. Furthermore, the exact cause of Kawasaki disease is not known. The inventors hypothesize that the cause of Kawasaki disease is a ubiquitous pathogen. Previously, the inventors discovered that serum samples from of KD patients taken in different geographic locations and from different times in history contained antibodies directed to similar antigens, supporting the inventors' hypothesis.

In a first aspect, antibodies or antigen binding fragments thereof are provided.

An isolated antibody or antigen binding fragment thereof of the present invention may comprise: a heavy chain variable domain comprising: a CDRH1 region selected from the group consisting of SEQ ID NOs: 2, 14, 22, 30, 42, 50, 62, 78, 86, 93, 101, 123, 126, 139, 149, 152, 164, 176, 186, and 205; a CDRH2 region selected from the group consisting of SEQ ID NOs: 74, 15, 23, 31, 35, 43, 51, 63, 70, 75, 79, 87, 94, 102, 111, 114, 127, 136, 140, 153, 165, 168, 177, 183, 187, 193, 206, and 212; a CDRH3 region selected from the group consisting of SEQ ID NOs: 4, 16, 14, 32, 36, 44, 52, 64, 71, 76, 80, 88, 95, 103, 112, 115, 124, 128, 137, 141, 150, 154, 166, 169, 178, 184, 188, 194, 207, 210, and 213; and a light chain variable domain comprising: a CDRL1 region selected from the group consisting of SEQ ID NOs: 6, 10, 18, 26, 37, 46, 54, 58, 66, 38, 82, 97, 105, 46, 119, 129, 132, 143, 146, 156, 160, 171, 180, 190, 197, 201, and 215; a CDRL2 region selected from the group consisting of SEQ ID NOs: 7, 11, 27, 28, 47, 55, 59, 67, 39, 83, 98, 106, 120, 133, 157, 161, 198, 202, and 208; and a CDRL3 region selected from the group consisting of SEQ ID NOs: 8, 12, 20, 28, 39, 48, 56, 60, 68, 73, 84, 99, 107, 109, 117, 121, 130, 134, 144, 147, 158, 162, 172, 174, 181, 191, 199, 203, and 216.

The antibody or antigen binding fragment may comprise any of the monoclonal antibodies shown in Table 1 or Table 2.

The terms “antibody” or “antibody molecule” are used herein interchangeably and refer to immunoglobulin molecules or other molecules which comprise an antigen binding domain. The term “antibody” or “antibody molecule” as used herein is thus intended to include whole antibodies (e.g., IgG, IgA, IgE, IgM, or IgD), monoclonal antibodies, chimeric antibodies, humanized antibodies, and antibody fragments, including single chain variable fragments (ScFv), single domain antibodies, and antigen-binding fragments, genetically engineered antibodies, among others, as long as the characteristic properties (e.g., ability to bind antigens derived from Kawasaki disease) are retained. The term “antibody fragment” as used herein is intended to include any appropriate antibody fragment that displays antigen binding function, for example, Fab, Fab′, F(ab′)2, scFv, Fv, dsFv, ds-scFv, Fd, mini bodies, monobodies, and multimers thereof and bispecific antibody fragments.

The term “antibody” includes “antibody fragments” or “antibody-derived fragments” and “antigen binding fragments” which comprise an antigen binding domain. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), (see for instance Bird et al., Science 242, 423-426 (1988) and Huston et al., PNAS USA 85, 5879-5883 (1988)). Such single chain antibodies are encompassed within the term antibody unless otherwise noted or clearly indicated by context.

Antibodies can be genetically engineered from the CDRs, VH, VL, and monoclonal antibody sequences described herein into antibodies and antibody fragments by using conventional techniques such as, for example, synthesis by recombinant techniques or chemical synthesis. Techniques for producing antibody fragments are well known and described in the art.

One may wish to engraft one or more CDRs from the monoclonal antibodies described herein into alternate scaffolds. For example, standard molecular biological techniques can be used to transfer the DNA sequences encoding the antibody's CDR(s) to (1) full IgG scaffold of human or other species; (2) a scFv scaffold of human or other species, or (3) other specialty vectors. If the CDR(s) have been transferred to a new scaffold all of the previous modifications described can also be performed. For example, one could consult Biotechnol Genet Eng Rev, 2013, 29:175-86 for a review of useful methods.

The antibodies or antibody fragments can be wholly or partially synthetically produced. Thus, the antibody may be from any appropriate source, for example recombinant sources and/or produced in transgenic animals or transgenic plants. Thus, the antibody molecules can be produced in vitro or in vivo. The antibody or antibody fragment can be made that comprises all or a portion of a heavy chain constant region, such as an IgG1, IgG2, IgG3, IgG4, IgA1, IgA2, IgE, IgM or IgD constant region.

Furthermore, the antibody or antibody fragment can further comprise all or a portion of a kappa light chain constant region or a lambda light chain constant region. All or part of such constant regions may be produced wholly or partially synthetic. Appropriate sequences for such constant regions are well known and documented in the art.

The term “fragment” as used herein refers to fragments of biological relevance (functional fragment), e.g., fragments which can contribute to or enable antigen binding, e.g., form part or all of the antigen binding site or can contribute to the prevention of the antigen interacting with its natural ligands. Fragments in some embodiments comprise a heavy chain variable region (VH domain) and light chain variable region (VL) of the disclosure. In some embodiments, the fragments comprise one or more of the heavy chain complementarity determining regions (CDRHs) of the antibodies or of the VH domains, and one or more of the light chain complementarity determining regions (CDRLs), or VL domains to form the antigen binding site.

The term “complementarity determining regions” or “CDRs,” as used herein, refers to part of the variable chains in immunoglobulins (antibodies) and T cell receptors, generated by B-cells and T-cells respectively, where these molecules bind to their specific antigen. As the most variable parts of the molecules, CDRs are crucial to the diversity of antigen specificities generated by lymphocytes. There are three CDRs (CDR1, CDR2 and CDR3), arranged non-consecutively, on the amino acid sequence of a variable domain of an antigen binding site. Since the antigen binding sites are typically composed of two variable domains (on two different polypeptide chains, heavy and light chain), there are six CDRs for each antigen binding site that can collectively come into contact with the antigen. A single whole antibody molecule has two antigen binding sites and therefore contains twelve CDRs. For further example, sixty CDRs can be found on a pentameric IgM molecule.

Within the variable domain, CDR1 and CDR2 may be found in the variable (V) region of a polypeptide chain, and CDR3 includes some of V, and all of diversity (D, heavy chains only) and joining (J) regions. Since most sequence variation associated with immunoglobulins and T cell receptors is found in the CDRs, these regions are sometimes referred to as hypervariable regions. Among these, CDR3 shows the greatest variability as it is encoded by a recombination of VJ in the case of a light chain region and VDJ in the case of heavy chain regions. The tertiary structure of an antibody is important to analyze and design new antibodies.

The human V_{H} complex is composed of approximately 100 gene segments per haploid genome, including at least 51 functional genes, as judged by successful rearrangement in cloned cDNA. On the basis of nucleic acid sequence homology, the V_{H} genes have been grouped into 6-7 families (VH 1-7). Among the seven families, the VH3 family is the largest. In some aspects the antibodies disclosed herein are derived from the VH3-74 family or the VH3-33 family or its paralog.

In some embodiments, the present invention comprises a polypeptide or protein comprising the antigen binding regions of the antibodies described herein, e.g., the CDRs (1-3) of the heavy and light chain that form the antigen binding region. The peptide may further comprise a detectable tag or other molecules.

As used herein, the terms “proteins” and “polypeptides” are used interchangeably herein to designate a series of amino acid residues connected to the other by peptide bonds between the alpha-amino and carboxy groups of adjacent residues. The terms “protein” and “polypeptide” refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing. The antibodies of the present invention are polypeptides, as well the antigen-binding fragments and fragments thereof.

The terms “monoclonal antibody” or “monoclonal antibody composition” as used herein refer to a preparation of antibody molecules of a single amino acid composition that specifically binds to a single epitope of the antigen.

The term “chimeric antibody” refers to an antibody comprising a variable region, i.e., binding region, from one source or species and at least a portion of a constant region derived from a different source or species, usually prepared by recombinant DNA techniques. Other forms of “chimeric antibodies” are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. In some embodiments, the antibodies are chimeric antibodies including heavy chain constant domains from non-human mammals (e.g., mouse, rat, rabbit, or non-human primate). In some embodiments, the antibodies disclosed in the present invention are chimeric antibodies including constant regions from rabbit heavy chain immunoglobulin sequences. Suitable heavy chain constant region sequences from non-human mammals, including mouse, rat, rabbit, and non-human primate are known in the art.

The antibodies disclosed in the present invention are human antibodies, as they include the constant region from human germline immunoglobulin sequences. However, the human CDRs or heavy and light variable chains may be used to make recombinant human antibodies or chimeric antibodies by recombinant means. The term “recombinant human antibody” includes all human antibodies that are prepared, expressed, created, or isolated by recombinant means, such as antibodies isolated from a host cell such as an SP2-0, NS0 or CHO cell (like CHO K1) or from an animal (e.g., a mouse) that is transgenic for human immunoglobulin genes or antibodies expressed using a recombinant expression vector transfected into a host cell. Such recombinant human antibodies have variable and, in some embodiments, constant regions derived from human germline immunoglobulin sequences in a rearranged form. In some embodiments, the antibodies are chimeric antibodies and the heavy chain constant domain is from rabbit, mouse, rat, or nonhuman primate.

ICI and KD specific monoclonal antibodies described herein include the following (Tables 1 and 2). The sequences referenced in Table 1 are nucleotide sequences, whereby the nucleotide sequences encode for the amino acid sequences of the light or heavy chain variable regions in Table 2. Sequence names ending in “L” or “K” indicate a nucleotide sequence that encodes a light chain variable domain and the remainder of the sequence names indicate a nucleotide sequence that encodes a heavy chain variable domain. The light chain constant domain may be a kappa light chain constant domain or a lambda light chain constant domain.

TABLE 1

| Antibody | Chain | Nucleotide Sequence | SEQ ID NO: |
|---|---|---|---|
| KD7-2H5 | Heavy | GAGGTGCAGCTGGTGGAGTCCGGGGGAG | 217 |
| | | GCTTAGTTCAGCCTGGGGGGTCCCTGAG | |
| | | ACTCTCCTGTGCAGCCTCTGGATTCACCT | |
| | | TCAGTAGCTACTGGATGCACTGGGTCCGC | |
| | | CAAGCTCCAGGGAAGGGGCTGGTGTGGG | |
| | | TCTCACGTATTAATAGTGATGGGAGTAGC | |
| | | ACAAGCTACGCGGACTCCGTGAAGGGCC | |
| | | GATTCGCCATCTCCAGAGACAACGCCAA | |
| | | GAACACGCTGTATCTGCAAATGAACAGT | |
| | | CTGAGAGCCGAGGACACGGCTGTGTATT | |
| | | ACTGTGCAAGAGCTTTGGGTGGCTGGGA | |
| | | TATTGACTACTGGGGCCAGGGAACCCTG | |
| | | GTCACCGTCTCG | |
| KD7-2H5L | Light | CAGTCTGTGCTGACGCAGCCACCCTCAGC | 218 |
| | | GTCTGGGACCCCCGGGCAGAGGGTCACC | |
| | | ATCTCTTGTTCTGGAAGCAGCTCCAACAT | |
| | | CGGAAGTAATTATGTATACTGGTACCAG | |
| | | CAGCTCCCAGGAACGGCCCCCAAACTCC | |
| | | TCATCTATAGGAATAATCAGCGGCCCTCA | |
| | | GGGGTCCCTGACCGATTCTCTGGCTCCAA | |
| | | GTCTGGCACCTCAGCCTCCCTGGCCATCA | |
| | | GTGGGCTCCGGTCCGAGGATGAGGCTGA | |
| | | TTATTACTGTGCAGCATGGGATGACAGCC | |
| | | TGAGTGGTGGAGTGTTCGGCGGAGGGAC | |
| | | CAAGCTGACCGTCCTAGGT | |
| KD10-1C3L | Light | CAGTCTGTGCTGACGCAGCCGCCCTCAGT | 219 |
| | | GTCTGCGGCCCCGGGACAGAAGGTCACC | |
| | | ATCTCCTGCTCTGGAGGCAGCTCCTCCAT | |
| | | TGGGAATAATTATGTATCCTGGTACCAAC | |
| | | TGCTCCCAGGAACAGCCCCCAAACTCCT | |
| | | CATTTATGACAATAATAAGCGACCCTCA | |
| | | GGGATTCCTGACCGATTCTCTGGCTCCAA | |
| | | GTCTGGCACGTCAGCCACCCTGGGCATC | |
| | | ACCGGACTCCAGACTGGGGACGAGGCCG | |
| | | ATTATTACTGCGGAACATGGGATAGCGG | |
| | | CCTGAGTGCTGGGGTGTTCGGCGGAGGG | |
| | | ACCAAGCTGACCGTCCTAGGT | |
| KD10-1C3 | Heavy | GTGCAGCTGGTGGAGTCTGGGGGAGGCG | 220 |
| | | TGGTCCAGCCTGGGAGGTCCCTGAGACT | |
| | | CTCCTGTGCAGCCTCTGGATTCACCTTCA | |
| | | GTGACTATGCTGTGCACTGGGTCCGCCAG | |
| | | GCTCCAGGCAAGGGGCTGGAGTGGGCGG | |
| | | CACTTATATCAAATGATGGAGATTTTAAA | |
| | | TATTACGCAGACGCCGTGAAGGGCCGAT | |
| | | TCACCATCTCCAGAGACAATTCCAACAA | |
| | | CACGCTGTATCTACAAATGAACAGCCTG | |
| | | AGAACTGAGGACACGGCTGTGTATTACT | |
| | | GTGCGAGAGAAAGAGGGAGGGATAAAG | |
| | | TATTCCCGCCGGGCTACTGGGGCCAGGG | |
| | | AACCCTGGTCACCGTCTCG | |
| KD7-1B5L | Light | CAGTCTGTGCTGACTCAGCCGCCCTCAGT | 221 |
| | | GTCTGCGGCCCCAGGACAGAAGGTCACC | |
| | | ATCTCCTGCTCTGGAAGCAGCTCCAACAT | |
| | | TGGGAATAATTATGTATCCTGGTACCAGC | |
| | | AGCTCCCAGGAACAGCCCCCAAACTCCT | |
| | | CATTTATGACAATAATAAGCGACCCTCA | |
| | | GGGATTCCTGACCGATTCTCTGGCTCCAA | |
| | | GTCTGGCACGTCAGCCACCCTGGGCATC | |
| | | ACCGGACTCCAGACTGGGGACGAGGCCG | |
| | | ATTATTACTGCGGAACATGGGATAGCAG | |
| | | CCTGAGTGCTGAGGTGTTCGGCGGAGGG | |
| | | ACCAAGCTGACCGTCCTAGGT | |
| KD7-1B5 | Heavy | GTGCAGCTGGTGGAGTCTGGGGGAGGCG | 222 |
| | | TGGTCCAGCCTGGGAGGTCCCTGAGACT | |
| | | CTCCTGTGAAGCGTCTGGATTCACCTTCA | |
| | | GGAGTCGTGCCATGCACTGGGTCCGCCA | |
| | | GGCTCCAGGCAAGGGGCTGGGGTGGGTG | |
| | | GCAGTTATATGGTACGATGGAAGTAATA | |
| | | AATACTATGCAGACTCCGTGAAGGGCCG | |
| | | ATTCACCATCTCCAGAGACAATTCCAGG | |
| | | AACACGCTGTATCTGCAAATGAACAGCC | |
| | | TGAGAGCCGAGGACACGGCTGTATATTA | |
| | | CTGTGCGAGAGATTTGGGTAGTGGTTTTT | |
| | | CCCTTGACTACTGGGGCCAGGGAACCCT | |
| | | GGTCACCGTCTCG | |
| KD6-1A8L | Light | CAGGCTGTGGTGACTCAGGAGCCCTCAC | 223 |
| | | TGACTGTGTCCCCAGGAGGGACAGTCAC | |
| | | TCTCACCTGTGCCTCCAGCACTGGAGCTG | |
| | | TCACCAGTGCTCATTCTCCCCACTGGTTC | |
| | | CGACAGAAGCCTGGCCAAGCCCCCAGGA | |
| | | CACTGATTTATGATACATCCAACAAACCG | |
| | | TCCTGGACACCTGCCCGGTTCTCAGGCTC | |
| | | CCTCCTTGGGGGCAAAGCTGCCCTGACCC | |
| | | TTTCGGGCGCGCAGCCTGAGGATGAGGC | |
| | | TGAGTATTACTGCTTGCTCTCCAATAGTG | |
| | | GAGTCCATTTTCTATTCGGCGGGGGGACC | |
| | | AGGTTGACCGTCCTAGGTCA | |
| KD6-1A8 | Heavy | GAGGTGCAGCTGGTGCAGTCTGGGGCTG | 224 |
| | | AGGTGAAGAAGACTGGGTCCTCAGTGAA | |
| | | GCTCTCCTGCACGGCTACCGGATACACTT | |
| | | TCACCTATCGCTACCTGCACTGGGTGCGA | |
| | | CAGGCCCCCGGACAAGCACTTGAGTGGA | |
| | | TGGGCTACATAACAATTTACAATGGTGA | |
| | | CACCAATTACGCACAGAAATTCCAGGAC | |
| | | AGAGTCACCATTTCCAGGGACATGTCTCT | |
| | | GAGCACAGTCTACATGGAGCTGAGCAGC | |
| | | CTGACATCAGAGGACACGGCCATGTATT | |
| | | TCTGTGTAAGATCCGCATTGTATGGGGAA | |
| | | AATGCTTTTGATTTTTGGGGCCAAGGGAC | |
| | | AATGGTCACCGTCTCA | |
| KD1-1H8 | Heavy | GAGGTGCAGCTGGTGGAGTCGGGGGGAG | 225 |
| | | GCTCAGTTCAGCCGGGGGGGTCCCTGAG | |
| | | ACTCTCCTGTGCAGCCTCTGGATTCACCT | |
| | | TCAGTAACTACTGGATGCACTGGGTCCGC | |
| | | CAAGCTCCAGGGAAGGGGCTGGTGTGGG | |
| | | TCTCACGTCTTAATATTGATGGGAGTAAC | |
| | | ACATTCTACGCGGACTCCGTCAAGGGCC | |
| | | GATTCACCATCTCCAGAGACAACGCCAA | |
| | | GAACACGCTGTATCTGCAAATGAACAGT | |
| | | CTGAGAGCCGAGGACACGGCTGTGTATT | |
| | | ACTGTGTAAGAGGAGGGAGTGACTCCGG | |
| | | TGACTCCGTTCCCTTCGATCTCTGGGGCC | |
| | | GTGGCACCCTGGTCACCGTCTCG | |
| KD1-1H8K | Light | GAAATAGTGATGACGCAGTCTCCAGCCA | 226 |
| | | CCCTGTCTGTGTCTCCAGGGGAAAGGGC | |
| | | CACCCTCTCCTGCAGGGCCAGTCAGAGT | |
| | | GTTAGCAGCAACTTAGCCTGGTACCAGC | |
| | | AGAACCCTGGCCAGGCTCCCAGGCTCCT | |
| | | CATCTATGGTGCATCCACCAGGGCCACTG | |
| | | GTATCCCAGCCAGGTTCAGTGGCAGTGG | |
| | | ATCTGGGACAGAGTTCACTCTCACCATCA | |
| | | GCAGCCTGCAGTCTGAAGATTTTGCAGTT | |
| | | TATTACTGTCAGCACTATAATAACTGGCC | |
| | | GTTCACTTTTGGCCAGGGGACCAAGGTG | |
| | | GAAATCAAAC | |
| KD1-2B1 | Heavy | GAGGTGCAGCTGGTGGAGTCCGGGGGAG | 227 |
| | | GCTTAGTTCAGCCTGGGGGGTCCCTGAG | |
| | | ACTCTCCTGTGCAGCCTCTGGATTCACCT | |
| | | TCAGTAGCTACTGGATGCACTGGGTCCGC | |
| | | CAAGCTCCAGGGAAGGGGCTGGTGTGGG | |
| | | TCTCACGTATTAATAATGATGGGAGTAGC | |
| | | ACAGGCTACGCGGACTCTGTGGAGGGCC | |
| | | GATTCACCATCTCCAGAGACAACGCCAA | |
| | | GAACACGCTGTATCTGCAAATCAACAGT | |
| | | CTGAGAGCCGAGGACACGGCTGTGTATT | |
| | | ACTGTGCAAGACAAGATGGTCACTACTA | |
| | | CTACAGTATGGACGTCTGGGGCCAAGGG | |
| | | ACCACGGTCACCGTCTC | |
| KD1-2B1L | Light | CAGTCTGCCCTGACTCAGCCTCGCTCAGT | 228 |
| | | GTCCGGGTCTCCTGGACAGTCAGTCACCA | |
| | | TCTCCTGCACTGGAACCAGCAGTGATGTT | |
| | | GGTGGTTATAACTATGTCTCCTGGTACCA | |
| | | ACAGCACCCAGGCAAAGCCCCCAAACTC | |
| | | ATGATTTATGATGTCAGTGAGCGGCCCTC | |
| | | AGGGGTCCCTGCTCGCTTCTCTGGCTCCA | |
| | | AGTCTGGCAACACGGCCTCCCTGACCATC | |
| | | TCTGGGCTCCAGGCTGAGGATGAGGCTG | |
| | | ATTATTACTGCTGCTCATATGCAGGCAAC | |
| | | TTCCGTTGGGTGTTCGGCGGAGGGACCA | |
| | | AGCTGACCGTCCTAGG | |
| KD10-1G3 | Heavy | GAGGTGCAGCTGGTGGAGTCTGGGGGAG | 229 |
| | | GCTTGGTCCAGCCGGGGGGGTCCCTGAA | |
| | | ACTCTCCTGTGCAGCCTCTGGGTTCAGTT | |
| | | TCAGTGGCGCTGCGATGCACTGGGTCCG | |
| | | CCAGTCCTCCGGGAGAGGGCTTGAGTGG | |
| | | CTTGGCCGTATTAGAAGCAAAACTAACG | |
| | | ACTATGCGACAGCATATGCAGAGTCGCT | |
| | | GCACGGCAGGTTCACCATCTCCAGAGAT | |
| | | GATGCAAAGAACACGGCGTATCTACAAA | |
| | | TGAACAGGCTGAAAAGCGAGGACACGGC | |
| | | CATATATTATTGTACAACCGTCTTGAGTA | |
| | | AGGGAGATCATGCGGTCTGGTTGGGCCC | |
| | | CTGGGGCCCGGGAACCCTGGTCACCGTC | |
| | | TC | |
| KD10-1G3L | Light | CAGCCTGTGCTGACTCAGCCACCTTCCTC | 230 |
| | | CTCCGCGTCTCCTGGAGAATCCGCCAGAC | |
| | | TCACCTGCACCTTGCCCAGTGACATCAGT | |
| | | GTTGCTGCGTCTGACATTTATTGGTATCA | |
| | | ACAGAAGGCAGGGAGCCCTCCCAACTTT | |
| | | CTCCTCTACGACCCGTCAGACTCACATAA | |
| | | GGGCCAGGACTCTGGAGTCCCCAGCCGC | |
| | | TTCTCTGGATCCAGAGATGGATCAGCCA | |
| | | ATTCAGGGTTTTTACTGATTTCCGGGGTC | |
| | | CAGTCTGAGGATGAGGCTGACTATTACT | |
| | | GCATGGTCTGGCCACCCAATACTGTGGGT | 231 |
| | | GTCGTCTTCGGCGGAGGGACCACTCTGA | |
| | | CCGTC | |
| KD5-2D1L | Light | CAGGCTGTGGTGACTCAGGAGCCCTCAC | |
| | | TGACTGTGTCCCCAGGAGGGACAGTCAC | |
| | | TCTCACCTGTGCTTCCAGCACTGGAGCAG | |
| | | TCACCAGTGGTTACTATCCAAACTGGTTC | |
| | | CAGCAGAAACCTGGACAAGCACCCAGGG | |
| | | CACTGATTTATAGTACAAGCAACAAACA | |
| | | CTCCTGGACCCCTGCCCGGTTCTCAGGCT | |
| | | CCCTCCTTGGGGGCAAAGCTGCCCTGAC | |
| | | ACTGTCAGGTGTGCAGCCTGAGGACGAG | |
| | | GCTGAGTATTACTGCCTGCTCTTCTATGG | |
| | | TGGTGTTCATGTCTTCGGAGCTGGGACCA | |
| | | AGGTCACCGTCCTAGGT | |
| KD5-2D1 | | GAGGTGCAGCTGGTGGAGTCCGGGGGAG | 232 |
| | | ACTTAGTTCAGCCGGGGGGGTCCCTAAG | |
| | | ACTCTCCTGTGCAGCCTCTGGATTCACCT | |
| | | TCAGTACCTACTGGATGCACTGGGTCCGC | |
| | | CAAGCTCCCGGGCAGGGGCTGGTGTGGG | |
| | | TCTCACGTATTAATGGTAATGGGAGAATC | |
| | | ACAAACTACGCGGACTCCGTGAAGGGCC | |
| | | GATTCACCGTCTCCCGAGACAACGCCAA | |
| | | GAACACGGTGGATCTGCAAATGAACAGT | |
| | | CTGAGAGCCGAGGACACGGCTGTGTATT | |
| | | ACTGTGCAAGAATCAATGAATGGGGAGA | |
| | | CGTCTGGGGCAAAGGGACCACGGTCACC | |
| | | GTCTCG | |
| KD10-1A8K | Light | GATATTGTGATGACCCAGACCCCACTCTC | 233 |
| | | TCTGTCCGTCACCCCTGGACAGCCGGCCT | |
| | | CCATCTCCTGCAAGTCTAGTCAGAGCCTC | |
| | | CTGCATAATGATGGAAAGACCTTTTTGCA | |
| | | TTGGTACCTGCAGAAGCCAGGCCAGTCT | |
| | | CCACAGCTCCTAATCTATGAAGTTTCCAG | |
| | | CCGCTTCTCTGGAGTGCCAGATAGGTTCA | |
| | | GTGGCAGCGGGTCAGGGACAGATTTCAC | |
| | | ACTGAAAATCAGCCGGGTGGAGGCTGAG | |
| | | GATGTTGGAGTTTATTACTGCATGCAAGG | |
| | | TATACACCTTCCTCCCACTTTCGGCGGGG | |
| | | GGACCAAGGTGGAAATCAAACGT | |
| KD10-1A8 | Heavy | GAGGTGCAGCTGGTGGAGTCCGGGGGAG | 234 |
| | | GCTTAGTTCAGCCGGGGGGGTCCCTGAG | |
| | | ACTCTCCTGTGCAGCCTCTGGATTCACAT | |
| | | TCAGTAGTTACTGGATGGAATGGGTCCG | |
| | | CCAAGCTCCAGGGAAGGGGCTGGTTTGG | |
| | | GTCTCACATATTAGTAGTGATGGGAGTGT | |
| | | TACAAGGTACGTGGACTCCGTGAAGGGC | |
| | | CGATTCACCATCTCCAGAGACAACGCCA | |
| | | AGAACACGCTGTATCTGCAAATGAATAG | |
| | | TCTGAGAGCCGAGGACACGGGTGTATAT | |
| | | TATTGTGCAAAAGATCTTCACTGGAACGC | |
| | | TCTTGATGTGTGGGGCCAAGGGACAATG | |
| | | GTCACCGTCTCG | |
| KD5-2D10K | Light | GAAATAGTGATGACGCAGTCTCCAGCCA | 235 |
| | | CCCTGTCTGTGTCTCCAGGGGAAAGAGC | |
| | | CACCCTCTCCTGCAGGGCCAGTCAGAGT | |
| | | GTTAGCAGCAACTTAGCCTGGTACCAGC | |
| | | AGAAACCTGGCCAGGCTCCCAGGCTCCT | |
| | | CATCTATGGTGCATCCACCAGGGCCACTG | |
| | | GTATCCCAGCCAGGTTCAGTGGCAGTGG | |
| | | GTCTGGGACAGAGTTCACTCTCACCATCA | |
| | | GCAGCCTGCAGTCTGAAGATTTTGCAGTT | |
| | | TATTACTGTCAGCAGTATAATAACTGGCC | |
| | | TCCGTGGACGTTCGGCCAAGGGACCAAG | |
| | | GTGGAAATCAAACGT | |
| KD5-2D10 | Heavy | GAGGTGCAGCTGGTGGAGTCCGGGGGAG | 236 |
| | | GCTTAGTTCAGCCTGGGGGGTCCCTGAG | |
| | | ACTCTCCTGTGCAGCCTCTGGATTCACCT | |
| | | TCAGTAGCTACTGGATGCACTGGGTCCGC | |
| | | CAAGCTCCAGGGAAGGGGCTGGTGTGGG | |
| | | TCTCACGTATTAATAGTGATGGGAGTAGC | |
| | | ACAAGCTACGCGGACTCCGTGAAGGGCC | |
| | | GATTCACCATCTCCAGAGACAACGCCAA | |
| | | GAACACGCTGTATCTGCAAATGAACAGT | |
| | | CTGAGAGCCGAGGACACGGCTGTGTATT | |
| | | ACTGTGCAAGAGGAGTCCGCAGTGGCTG | |
| | | GTACGCTGATGCTTTTGATATCTGGGGCC | |
| | | AAGGGACAATGGTCACCGTCTCG | |
| KD5-2D7 | Heavy | GAGGTGCAGCTGGTGGAGTCCGGGGGAG | 237 |
| | | GCTTAGTTCAGCCGGGGGGGTCCCTGGG | |
| | | ACTCTCCTGTGCAGCCTCTGGATTCACCT | |
| | | TCAGTAACTACTGGATGCACTGGGTCCGC | |
| | | CAAGCTCCAGGGAAGGGGCTGGTGTGGG | |
| | | TCTCGCGTATTAATAGTGATGGGAGTGAC | |
| | | ACAAGCTACGCGGACTCCGTGAAGGGCC | |
| | | GATTCACCATCTCCAGAGACAACGCCGA | |
| | | GAACACGCTGTATCTGCACATGAGCAGT | |
| | | CTGAGAGCCGAGGACACGGCTGTCTATT | |
| | | ACTGTGCAAGGGAATTGGGCTACGGTGA | |
| | | CTACGGTATGGACGTCTGGGGCAAAGGG | |
| | | ACCACGGTCACCGTCTCG | |
| KD5-2D7L | Light | CAGTCTGTGCTGACGCAGCCACCCTCAGC | 238 |
| | | GTCTGGGACCCCCGGGCAGAGGGTCACC | |
| | | ATCTCTTGTTCTGGAAGCAGCTCCAACAT | |
| | | CGGAAGTCATTCTGTGATCTGGTACCAGC | |
| | | AGCTCCCAGGAACGGCCCCCAAACTCCT | |
| | | CGTCTATAGTGATAATCAGCGGCCCTCAG | |
| | | GGGTCCCTGACCGATTCTCTGGCTCCAAG | |
| | | TCTGGCACCTCAGCCTCCCTGGCCATCAG | |
| | | TGGGCTCCAGTCTGAGGATGAGGCTGAT | |
| | | TATTACTGTGCAGCATGGGATGACAGCCT | |
| | | GAATCATCTTCATGTGGTATTCGGCGGAG | |
| | | GGACCAAGCTGACCGTCCTA | |
| KD10-2F6 | Heavy | GAGGTGCAGCTGGTGGAGTCCGGGGGAG | 239 |
| | | GCTTAGTTCAGCCTGGGGGGTCCCTGAG | |
| | | ACTCTCCTGTGCAGCCTCCGGATTCATCT | |
| | | TCAGTAACTACTGGATGCACTGGGTTCGC | |
| | | CAAGCTCCAGGGGAGGGGCTGGTGTGGG | |
| | | TCTCACGTATTAATAAAGATGGGAGTAG | |
| | | CACATTTTACGCGGACTCCGTGAAGGGC | |
| | | CGATTCACCATCTCCAGAGACAACGCCA | |
| | | AGAACACGCTGTATCTGCAAATGAACAG | |
| | | TCTGAGAGCCGAGGACACGGCTGTGTAT | |
| | | TATTGTACAAGAGATTTCGATTTTTGGAG | |
| | | TGGCTACTGGGGCCAGGGAACCCTGGTC | |
| | | ACCGTCTCG | |
| KD10-2F6L | Light | CAGTCTGCCCTGACTCAGCCTGCCTCCGT | 240 |
| | | GTCTGGGTCTCCTGGACAGTCGATCACCA | |
| | | TCTCCTGCACTGGAACCAGCAGTGACGTT | |
| | | GGTGGTTGTGAGTATGTCTCCTGGTACCA | |
| | | ACAGCACCCAGGCAAAGTCCCCAAACTC | |
| | | ATCATTTATGAGGTCAGTAATCGGCCCTC | |
| | | AGGGGTTTCTAATCGCTTCTCTGGCTCCA | |
| | | AGTCTGGCAACACGGCCTCCCTGACCGTC | |
| | | TCTGGGCTCCAGGCTGAGGACGAGGCTG | |
| | | ATTATTTCTGCAGCTCCTGTACAACCAGC | |
| | | GGCTCTTATGTCTTCGGAGCTGGGACCAA | |
| | | GGTCACCGTCGTA | |
| KD11-2E4 | Heavy | GAGGTGCAGCTGGTGGAGTCCGGGGGAG | 241 |
| | | GCGTAGTTCAGCCTGGGGAGTCCCTGAG | |
| | | ACTCTCCTGTGCAGCCTCCGGAGTCACCT | |
| | | TCAGTAGTTACTGGATGCATTGGGTCCGC | |
| | | CAAGTTCCAGGGAAGGGGCTGGTGTGGG | |
| | | TCGCACGTATTAATATTGATGGGACCAGT | |
| | | ACAACCTACGCGGACTGTGTGAAGGGCC | |
| | | GATTCACCATCTCCAGAGACAACGCCAA | |
| | | GAACACGCTGTATCTGCAAATGAGCAGT | |
| | | CTGAGAGCCGAGGACACGGCTGTTTATT | |
| | | ACTGTGCAACAGAGCGAGGATTACTTAG | |
| | | TGGTGGTCGCTGGCACTCCTCCCACTTTG | |
| | | ACTACTGGGGCCAGGGAACCCTGGTCAC | |
| | | CGTCTCG | |
| KD11-2E4K | | GACATCCAGATGACCCAGTCTCCTTCCAC | 242 |
| | | CCTGTCTGCATCTGTAGGAGACAGAGTC | |
| | | ACCATCACTTGCCGGGCCAGTCAGAGTA | |
| | | TTAGTAGCTGGTTGGCCTGGTATCAGCAG | |
| | | AAACCAGGGAAAGCCCCTAAGGTCCTGA | |
| | | TCTATAAGACGTCTAGTTTAGAAAGTGG | |
| | | GGTCCCATCAAGGTTCAGCGGCAGTGGC | |
| | | TCTGGGACAGAATTCACTCTCACCATCAG | |
| | | CAGCCTGCAGCCTGATGATTTTGCAACTT | |
| | | ATTACTGCCAACAGTATGACAGTTCTTCT | |
| | | CTCACGTGGACGTTCGGCCAAGGGACCA | |
| | | AGGTGGAAATCAAAC | |
| KD11-2E5 | Heavy | GAGGTGCAGCTGGTGGAGTCCGGGGGAG | 243 |
| | | GCTTAGTTCAGCCTGGGGGGTCCCTGAG | |
| | | ACTCTCCTGTGCAGCCTCTGGATTCACCT | |
| | | TCAGTAGGTTCTGGATGCACTGGGTCCGC | |
| | | CAGGTTCCAGGGAAGGGGCTGGTGTGGA | |
| | | TCTCACGTATTAATAGTGATGCGACTAGC | |
| | | TCAAGCTACGCGGACTCCGTGAAGGGCC | |
| | | GATTCACCATCTCCAGAGACAACGCCAA | |
| | | GAACACACTGTTTCTGCAAATGAACAGT | |
| | | CTGAGAGCCGAGGACACGGCTGTCTATT | |
| | | ACTGTGCAACCAGCAATACAGCTCTGGTT | |
| | | TATTTGCCTGAGAACTGGGGCCAGGGAA | |
| | | CCCTGGTCACCGTCTCG | |
| KD11-2E5K | | GAAATTGTGTTGACACAGTCTCCAGCCAC | 244 |
| | | CCTGTCTTTGTCTCCAGGAGAGAGAGCCA | |
| | | CCCTCTCCTGCAGGGCCAGTCAGAGTGTT | |
| | | AGCGACTACTTAGCCTGGTACCAACAGA | |
| | | AACCTGGCCAGGCTCCCAGGCTCCTCATC | |
| | | TATGATGCATCCAACAGGGCCACTGGCA | |
| | | TCCCAGCCAGGTTCAGTGGCGGTGGGTCT | |
| | | GGGACAGACTTCACTCTCACCATCAGCA | |
| | | GCCTAGAGCCTGAAGATTTTGCAGTTTAT | |
| | | TACTGTCAGCAGCGCAGCTACTGGCCTCC | |
| | | GACGTTCGGCCAAGGGACCAAGGTGGAA | |
| | | ATCAAAC | |
| KD2-1D10L | Light | CAGTCTGCCCTGACTCAGCCTCCCTCCGC | 245 |
| | | GTCCGGGTCTCCTGGACAGTCAGTCACCA | |
| | | TCACCTGCACTGGAACCAGCAGTGACGT | |
| | | TGGTGGTTATAACTATGTCTCCTGGTACC | |
| | | AACAGCACCCAGGCAAAGCCCCCAAACT | |
| | | CATGATTTATGAGGTCAGTAAGCGGCCCT | |
| | | CAGGGGTCCCTGATCGCTTCTCTGGCTCC | |
| | | AAGTCTGGCAACACGGCCTCCCTGACCG | |
| | | TCTCTGGGCTCCAGGCTGAGGATGAGGC | |
| | | TGATTATCACTGCAGCTCATTTGCAGGCG | |
| | | ACAACAATTCCCCGGTATTCGGCGGAGG | |
| | | GACCAAATTGACCGTCCTAG | |
| KD2-1D10 | Heavy | GAGGTGCAGCTGGTGGAGTCCGGGGGAG | 246 |
| | | GCTTAATTCACCCGGGGGGGTCCCTGAG | |
| | | ACTCTCCTGTGCAGCCTCTGGATTCACCT | |
| | | TCAGTAGCTACTGGATGCACTGGGTCCGC | |
| | | CAAGCCCCAGGGAAGGGGCTGGTGTGGG | |
| | | TCTCACATATTAAGAGTGATGGGAGTAA | |
| | | CACAATCTACGCGGACTCCGTGAAGGGC | |
| | | CGATTCACCATCTCCAGAGACAACGCCA | |
| | | AGAACACGCTGTATCTACAAATGAACAG | |
| | | TCTGAGAGCCGAGGACACGGCTGTGTAT | |
| | | TACTGTGGAAGAGGGCGCAGTTTGACCC | |
| | | CACGCTCGGCCATTGACTACTGGGGCCA | |
| | | GGGAACCCTGGTCACCGTCTC | |
| KD9-2A4 | Heavy | CTGGTGGAGTCTGGGGGAGGCTTGGTCC | 247 |
| | | AGCCTGGGGGGTCCCTGAGACTCTCCTGT | |
| | | GCAGCCTCTGGATTCACCTTCAGCAGTTA | |
| | | TGCTATGCACTGGGTCCGCCAGGCTCCAG | |
| | | GGAAGGGACTGGAGTTTGCTTCAGCTATT | |
| | | AGTAGTGATGGGGGTACCACATATTACG | |
| | | CAAACTCTGTGAAGGGCAGATTCACCAT | |
| | | CTCCAGAGACAATTCCAAGAACACGCTG | |
| | | TATCTTCAAATGGGCAGCCTGAGGGATG | |
| | | AAGACATGGCTGTGTATTACTGTGCGAG | |
| | | AGATGATCTGAGCACCAGCTGGGACCTT | |
| | | GACTACTGGGGCCAGGGAACCCTGGTCA | |
| | | CCGTCTC | |
| KD9-2A4L | Light | CAGTCTGCCCTGACTCAGCCTGCCTCCGT | 248 |
| | | GTCTGGGTCTCCTGGACAGTCGATCACCA | |
| | | TCTCCTGCACTGGAACCAGCAGTGACGTT | |
| | | GGTGGTTATAACTATGTCTCCTGGTACCA | |
| | | ACAGCACCCAGGCAAAGCCCCCAAACTC | |
| | | ATGATTTATGACGTCAGTAATCGGCCCTC | |
| | | AGGGGTTTCTAATCGCTTCTCTGGCTCCA | |
| | | AGTCTGGCAACACGGCCTCCCTGACCATC | |
| | | TCTGGGCTCCAGGCTGAGGACGAGGCTG | |
| | | ATTATTACTGCAGCTCATATACAATCAGC | |
| | | AGCACTAACGTCTTCGGAACTGGGACCA | |
| | | AGGTCACCGTCCTAG | |
| KD3-1B6L | Light | CAGTCTGCCCTGACTCAGCCTGCCTCCAT | 249 |
| | | GTCTGGGTCTCCTGGACAGTCGATCACCA | |
| | | TCTCCTGCACTGGAACCAGCAGTGATGTT | |
| | | GGAGCTCATAACTTTATCTCCTGGTACCA | |
| | | ACAACACCCAGGCAAAGCCCCCAAACTC | |
| | | ATGATTTTTGAGGTCAATAAGCGGCCCTC | |
| | | AGGGGTTTCTAATCGCTTCTCTGGCTCCA | |
| | | AGTCTGGCAACACGGCCTCCCTGACAAT | |
| | | CTCTGGCCTCCAGGCTGAGGACGAGGCT | |
| | | GATTATTTCTGCTGCTCATATGCAGGTTA | |
| | | TAGCACTTGGGTGTTCGGCGGAGGGACC | |
| | | AAGCTGACCGTCCTAG | |
| KD3-1B6 | Heavy | GAGGTGCAGCTGGTGGAGTCCGGGGGAG | 250 |
| | | GCGTAGTCCAGCCTGGGGGGTCCCTGAG | |
| | | ACTCTCCTGTGAAGTCTCTGGATTCATCT | |
| | | TCAGTAGCTACTGGATGCACTGGGTCCGC | |
| | | CAAGTTGCAGGGAAGGGGCTGGTGTGGG | |
| | | TCTCACGTATAAATAGTGATGGGAGTAG | |
| | | TACAAGTTACGCGGACTCCGTGAAGGGC | |
| | | CGATTCACCATCTCCAGAGACAACGCCA | |
| | | AGAATACGCTGAATCTGCAAATGAATTTT | |
| | | CTGAGAGCCGAGGACACGGCTGTGTATT | |
| | | ATTGTGCAAGAGGGGGTGATCACGGTGA | |
| | | CTACGGCTTCTTTGAGTCTTGGGGCCAGG | |
| | | GAACCCTGGTCACCGTCTC | |
| KD3-1C10 | Heavy | GAGGTGCAGCTGGTGGAGTCCGGGGGAG | 251 |
| | | GGTTAGTTCAGCCTGGGGGGTCCCTGAG | |
| | | ACTCTCCTGTGCAGCCTCTGGATTCACCT | |
| | | TCAGTAATCACTGGATGCACTGGGTCCGC | |
| | | CAAGCTCCAGGGAAGGGGCTGCTGTGGG | |
| | | TCTCGCGTATTGATACTGGTGGGAGTACC | |
| | | ACAAACTACGCGGACTCCGTGAAGGGCC | |
| | | GATTCACCATCTCCAGAGACAGCGCCAA | |
| | | GAACACGGTGTATCTACAAATGAACAGT | |
| | | CTGAGAGCCGAAGACACGGCTGTTTATT | |
| | | ACTGTGCAAGAGGCGGTCTCTTCTACTAC | |
| | | GGTATGGACGTCTGGGGCCAAGGGACCA | |
| | | CGGTCACCGTCTC | |
| KD3-1C10K | Light | GAAATTGTGTTGACGCAGTCTCCAGGCA | 252 |
| | | CCCTGTCTTTGTCTCCAGGGGAAAGGGCC | |
| | | ACCCTCTCCTGCAGGGCCAGTCAGACTA | |
| | | GTAGCAGCACCTCCTTAGCCTGGTATCAG | |
| | | CAGAAACGTGGCCAGGCTCCCAGGCTCC | |
| | | TCATCTATGGTGCATCCCGCAGGGCCACT | |
| | | GGCATCCCAGACAGGTTCAGTGGCAGTG | |
| | | GGTCTGGGACAGACTTCACTCTCACCATC | |
| | | AGCAGACTGGAGCCTGAAGATTTTGCAG | |
| | | TATATTACTGTCAGCAATTTAGTGGCTCA | |
| | | CCTGCGTACACTTTTGGCCAGGGGACCA | |
| | | AGGTGGAAATCAAAC | |
| KD6-2H8K | Light | GATATTGTGATGACCCAGACTCCACTCTC | 253 |
| | | CCTGCCCGTCACCCCTGGAGAGCCGGCC | |
| | | TCCATCTCCTGTAGGTCTAGTCAGAGCCT | |
| | | CTTGGATAGTGCTGATGGAAACACCTATT | |
| | | TGGACTGGTACCTGCAGAAGCCAGGGCA | |
| | | GTCTCCACAGCTCCTGATCCATACGCTTT | |
| | | CCCATCGGGCCTCTGGAGTCCCAGACAG | |
| | | GTTCAGTGGCAGTGGGTCAGGCACTGAT | |
| | | TTCACACTGAAAATCAGCAGGGTGGAGG | |
| | | CTGAAGATGTTGGAGTTTATTACTGCATG | |
| | | CAACGTATAGCCTTTCCCGTCACTTTCGG | |
| | | CCCTGGGACCAAAGTGGATATCAAAC | |
| KD6-2H8 | Heavy | GAGGTGCAGCTGGTGGAGTCCGGGGGAG | 254 |
| | | GCTTAGTTCAGCCTGGGGGGTCCCTGAG | |
| | | ACTCTCCTGTGCAGCCTCTGGATTCACCT | |
| | | TCAGTAACTACTGGATGCACTGGGTCCGC | |
| | | CAAGCTCCAGGGGAGGGGCTGGAGTGGG | |
| | | TCTCACGTATTAATAGTGATGGGAATAA | |
| | | GGTAAGTTACGCGGACTCCGTGAGGGGC | |
| | | CGATTCACCATCTCCAGAGACAACGCCA | |
| | | AGAACACGCTGTATCTACAAATGAACAG | |
| | | CCTGACAGGCGAGGACACGGCTGTGTAT | |
| | | TATTGTGCAAGATCTAACTGGGGATCGG | |
| | | CAGACTACTGGGGCCAGGGAACCCTGGT | |
| | | CACCGTCTC | |
| KD6-1H10 | Heavy | GAGGTGCAGCTGGTGGAGTCTGGGGGAG | 255 |
| | | GCTCAGTTCAGCCTGGGGGGTCCCTGAG | |
| | | ACTCTCCTGTGCTGCCTCTGGATTCACCT | |
| | | TCAGTAGATACTGGATGCACTGGGTCCG | |
| | | CCAGGCTCCAGGGGAGGGGCTCGTTTGG | |
| | | GTCTCACGTATAAATAGTGATGAGACTG | |
| | | ACAAGCTTTACGCGGACTCCGTGAAGGG | |
| | | CCGATTCTCCATCTTCAGAGACAACGCCA | |
| | | AGAACACACTATATCTGCAAATGAACAG | |
| | | ACTGAGAGCCGAGGACACGGCTGTATAC | |
| | | TACTGTGCAAGAGATCGAGAGGATGTTG | |
| | | TAGTGGGGCCAGCTACTCAACACACCAT | |
| | | CTTTAACTCCTGGGGCCAGGGAACCCTG | |
| | | GTCACCGTCTC | |
| KD6-1H10L | Light | CAGTCTGCCCTGACTCAGCCTGCCTCCGT | 256 |
| | | GTCTGGGTCTCCTGGACAGTCGATCACCA | |
| | | TCTCCTGCACTGGAACCAGCAGTGATGTT | |
| | | GGGAGTTCTAACTTTGTCTCCTGGTACCA | |
| | | ACAACACCCAGGCAAAGCCCCCAAACTC | |
| | | ATCCTTTATGAGGTCAGTAAGCGGCCCGC | |
| | | TGGAGTTTCTAGTCGCTTCTCTGGCTCCA | |
| | | GGTCTGGCAACACGGCCTCCCTGACAAT | |
| | | CTCTGGAGCCCAGGCTGAGGACGAGGCT | |
| | | GACTATTCCTGCTGCTCAACTTCTTCTGTT | |
| | | GGCACTCTTTATGTCTTCGGAACTGGGAC | |
| | | CAAGGTAACCGTCCTAG | |
| KD8-2E9L | Light | CAGTCTGTGCTGACGCAGCCGCCCTCAGT | 257 |
| | | GTCTGCGGCCCCAGGACAGAAGGTCACC | |
| | | ATCTCCTGCTCTGGAAGCAACTACAACAT | |
| | | TGGGAATAATTATGTATCCTGGTACCAGC | |
| | | GACTCCCAGGAACAGCCCCCAAACTCCT | |
| | | CATTTATGACAATAATAAGCGACCCTCA | |
| | | GGGATTCCTGACCGATTCTCTGGCTCCAA | |
| | | GTCTGGCACGTCAGCCACCCTGGGCATC | |
| | | ACCGGACTCCAGACTGGGGACGAGGCCG | |
| | | ATTATTACTGCGGAACATGGGATAGCAG | |
| | | CCTGAGGGCTGGGGTGTTCGGCGGAGGG | |
| | | ACCAAGCTGACCGTCCTAG | |
| KD8-2E9 | Heavy | GAGGTGCAGCTGGTGGAGTCCGGGGGAG | 258 |
| | | GCTTAGTTCAGCCTGGGGGGTCCCTGAG | |
| | | ACTCTCCTGTGCAGCCTCTGGATTCACTT | |
| | | TCAGTAGGCACTGGATGCACTGGGTCCG | |
| | | CCAAGCTCCAGGGAAGGGGCTGGTGTGG | |
| | | GTCTCACGTATTAATAGTGATGGGAGTA | |
| | | GCACAAGTAACGCGGACTCCGTGAAGGG | |
| | | CCGAATCACCATCTCCAGAGACAACGGC | |
| | | AAGAACACGCTGTATCTGCAGATGAACA | |
| | | GTCTGAGAGCCGAGGACACGGCTGTGTA | |
| | | TTACTGTGCAAGGGAGATAGCATCGGGG | |
| | | ACAGATGCTTTTGATATCTGGGGCCAAG | |
| | | GGACAATGGTCACCGTCTC | |
| KD9-2H6 | Heavy | GAGGTGCAGCTGGTGGAGTCTGGGGGAG | 259 |
| | | GCTTAGTAAAGCCTGGGGAGTCCCTTAG | |
| | | ACTCTCCTGTGTAGGTTCTGGATTCACTC | |
| | | TCACTAACGCCTGGATGATCTGGGTCCGC | |
| | | CAGACTTCAGGGAAGGGGCTGGAATGGG | |
| | | TTGGTCGCATCAAGAGTAAAATTGATGG | |
| | | TGGGGCAATCGACTACGGTGCACCCGTG | |
| | | AAAGGTAGATTTACCATCTCAAGAGATG | |
| | | ATACAAAAAACACGGTGTATCTGCAAAT | |
| | | GAACAGCCTGCAAACCGACGACACAGGC | |
| | | GTCTATTTCTGTACCACAGATCGTTATAG | |
| | | TACTGGCTACTACGGCATGGACGACTACT | |
| | | GGGGCCAGGGAACCCTGGTCACCGTCTC | |
| KD9-2H6L | Light | TCCTATGAGCTGACTCAGCCACCCTCAGT | 260 |
| | | GTCCGTGTCCCCAGGACAGACAGCCACC | |
| | | ATCACCTGCTCTGGAGATAAATTGGGAG | |
| | | ATAAATATTCTTTCTGGTATCAACAGAAG | |
| | | CCAGGCCAGTCCCCTGTGGTGGTCATCTA | |
| | | TCAAGATTCCAAGCGGCCCTCAGGGATC | |
| | | CCTGAGCGATTCTCTGGCTCCAACTCTGG | |
| | | GAACACAGCCACTCTGACCATCAGCGGG | |
| | | ACCCAGGCTATGGATGAGGCTGACTATT | |
| | | ACTGTCAGGTGTGGGACACCGACTCTGC | |
| | | AGTCTTCGGAACTGGGACCAGGGTCACC | |
| | | GTCCT | |
| KD12-1F10K | Light | GACATCCAGATGACCCAGTCTCCATCCTC | 261 |
| | | CCTGTCTGCATCTGTAGGAGACAGAGTC | |
| | | ACCATCACTTGCCGGGCAAGTCAGAGCA | |
| | | TTAGCAGCTATTTAAATTGGTATCAGCAG | |
| | | AAACCAGGGAAAGCCCCTAAGCTCCTGA | |
| | | TCTATGCTGCATCCAGTTTGCAAAGTGGG | |
| | | GTCCCATCAAGGTTCAGTGGCAGTGGAT | |
| | | CTGGGACAGATTTCACTCTCACCATCAGC | |
| | | AGTCTGCAACCTGAAGATTTTGCAACTTA | |
| | | CTACTGTCAACAGAGTTACAGTACCCCGT | |
| | | ACACTTTTGGCCAGGGGACCAAGGTGGA | |
| | | AATCAAAC | |
| KD12-1F10 | Heavy | GAGGTGCAGCTGGTGGAGTCTGGGGGAG | 262 |
| | | GCCTGGTCAAGCCTGGGGGGTCCCTGAG | |
| | | ACTCTCCTGTGCGGCCTCTGGATTCACCT | |
| | | TCAGTAGCTATAGCATGAACTGGGTCCG | |
| | | CCAGGCTCCAGGGAAGGGGCTGGAGTGG | |
| | | GTCTCATCCATTAGTAGTAGTAGTAGTTA | |
| | | CATATACTACGCAGACTCAGTGAAGGGC | |
| | | CGATTCACCATCTCCAGAGACAACGCCA | |
| | | AGAACTCACTGTATCTGCAAATGAACAG | |
| | | CCTGAGAGCCGAGGACACGGCTGTGTAT | |
| | | TACTGTGCGAGAGATACGATTTCTCGATA | |
| | | CTGGGGCCAGGGAACCCTGGTCACCGTC | |
| | | TC | |
| KD12-1A10 | Heavy | GAGGTGCAGCTGGTGGAGTCTGGGGGAG | 263 |
| | | GCCTGGTCAAGCCTGGGGGGTCCCTGAG | |
| | | ACTTTCCTGTGCAGCCTCTGGATTCACCT | |
| | | TCAGTAGTTACAGCATGAACTGGGTCCG | |
| | | CCAGGCTCCAGGGAAGGGGCTGGAGTGG | |
| | | GTCTCATCCATTAGTGGTAGTAGTAGTTA | |
| | | CATGCACTACGCAGAGTCAGTGAAGGGC | |
| | | CGATTCACCATCTCCAGAGACAACGCCA | |
| | | AGAACTCACTGTATCTGCAAATGAATAG | |
| | | CCTGAGAGCCGAGGACACGGCTGTGTAT | |
| | | TACTGTGCGAGTACGGGGATTATCACGT | |
| | | ATTACTATGCTTCGGGGGTCCCTGACTAC | |
| | | TGGGGCCAGGGAACCCTGGTCACCGTCT | 264 |
| | | C | |
| KD12-1A10L | Light | CAGTCTGTGCTGACTCAGCCACCCTCAGC | |
| | | GTCTGGGACCCCCGGGCAGAGGGTCACC | |
| | | ATGTCTTGTTCTGGAAGCAGCTCCAACAT | |
| | | CGGAAGAAATTATGTATACTGGTACCAG | |
| | | CAGCTCCCAGGAACGGCCCCCAAACTCC | |
| | | TCATCTATAGGAATAATCAGCGACCCTCA | |
| | | GGGGTCCCTGACCGATTCTCTGGCTCCAA | |
| | | GTCTGGCACCTCAGTCTCCCTGGCCATCA | |
| | | GTGGGCTCCGGTCCGAGGATGAGGCTGA | |
| | | TTATTACTGTGCAGCATGGGATGACAGCC | |
| | | TGAGTGGTGTGGTATTCGGCGGAGGGAC | |
| | | CAAGCTGACCGTCCTAG | |
| KD12-2A10K | Light | GAAATTGTGTTGACGCAGTCTCCAGGCA | 265 |
| | | CCCTGTCTTTGTCTCCAGGGGAAAGAGCC | |
| | | ACCCTCTCCTGCAGGGCCAGTCAGAGTGT | |
| | | TAGCAGCAACTTAGCCTGGTACCAGCAG | |
| | | AAACCTGGCCAGGCTCCCAGGGTCCTCA | |
| | | TCTATGGTGCATCCAGCAGGGCCACTGG | |
| | | CATCCCAGACAGGTTCAGTGGCAGTCGG | |
| | | TCTGGGACAGACTTCACTCTCACCATCAG | |
| | | CAGACTGGAGCCTGAAGATTTTGCAGTG | |
| | | TATTACTGTCAGCGGTATGATAACTCACC | |
| | | TCGGACGTTCGGCCAAGGGACCAAGGTG | |
| | | GAAATCAAAC | |
| KD12-2A10 | Heavy | GAGGTGCAGCTGGTGGAGTCTGGGGGAG | 266 |
| | | GCTTGGTAAAGCCTGGGGGGTCCCTTAG | |
| | | ACTCTCCTGTGCAGCCTCTGGTTTCACTT | |
| | | TCATTAACGCCTGGATGAACTGGGTCCGC | |
| | | CAGGCTCCAGGGAAGGGGCTGGAGTGGG | |
| | | TCGGCCTAATTAAAAGCAAAACTGATGG | |
| | | TGGGACAATAGACTACGCTGCACCCGTG | |
| | | AAAGGCAGATTCACTATTTCAAGAGATG | |
| | | ATTCAGAAAAAATGTTGTATCTGCAAAT | |
| | | GGACAGCCTGAAGACCGAGGACACAGCC | |
| | | GTGTATTACTGTACCACAATGTATGGCTG | |
| | | GAAGGACGCGAGGGACTATTGGGGCCAG | |
| | | GGAACCCTGGTCACCGTCTC | |
| KD12-2A1K | Light | GAAATTGTGTTGACGCAGTCTCCAGGCA | 267 |
| | | CCCTGTCTTTGTCTCCAGGGGAAAGAGCC | |
| | | ACCCTCTCCTGCAGGGCCAGTCAGAGTGT | |
| | | TAGCAGCAGCTACTTAGCCTGGTACCAG | |
| | | CAGAAACCTGGCCAGGCTCCCAGGTTCC | |
| | | TCATCTATGGTGCGTCCAGTAGGGCCACT | |
| | | GGCATCCCGGACAGGTTCAGTGGCAGTG | |
| | | GGTCTGGGACAGACTTCACTCTCACCATC | |
| | | AGCAGACTGGAGCCTGAAGATTTTGCAG | |
| | | TGTATTACTGTCAGCAGTATGGTAGCTCA | |
| | | CCGTACACTTTTGGCCAGGGGACCAAGG | |
| | | TGGAAATCAAAC | |
| KD12-2A1 | Heavy | GAGGTGCAGCTGGTGGAGTCCGGGGGAG | 268 |
| | | GCTTAGTTCAGCCTGGGGGGTCCCTGAG | |
| | | ACTCTCCTGTGCAGCCTCTGGATTCACCT | |
| | | TCAGTAGCTACTGGATGCACTGGGTCCGC | |
| | | CAAGCTCCAGGGAAGGGGCTGGTGTGGG | |
| | | TCTCACGTATTAAAAGTGATGGGAGTAG | |
| | | CACAAGCTACGCGGACTCCGTGAAGGGC | |
| | | CGATTCACCATCTCCAGAGACAACGCCA | |
| | | AGAACACGCTGTATCTGCAAATGAACAG | |
| | | TCTGAGAGCCGAGGACACGGCTGTGTAT | |
| | | TACTGTGCAAGAGGGGGTGGCAGCAGCA | |
| | | ACTGGTACCCGGGTTTCTTTGACTACTGG | |
| | | GGCCAGGGAACCCTGGTCACCGTCTC | |
| KD12-2H1 | Heavy | GAGGTGCAGCTGTTGGAGTCTGGGGGAG | 269 |
| | | GCTTGGTACAGCCGGGGGGGTCCCTGAG | |
| | | ACTCTCCTGTGTAGCCTCTGGATTCACCT | |
| | | TCAGCAGCTATGCCATGAGCTGGGTCCG | |
| | | CCAGGCTCCAGGGAAGGGGCTGGAGTGG | |
| | | GTCTCTAGTATTAGTGCTAGTGGTGGTAG | |
| | | CACATATTACGCAGACTCCGTGAAGGGC | |
| | | CGGTTCACCATCTCCAGAGACAATTCCAA | |
| | | GAACACGCTGTATCTGCAAATGAACAGC | |
| | | CTGAGAGCCGAGGACACGGCCGTATATT | |
| | | ACTGTGGGAATTGGCCGGAAGGATTCCC | |
| | | GGCCTACTTTCACTACTGGGGCCAGGGA | |
| | | ACCCTGGTCACCGTCTC | 270 |
| KD12-2H1K | Light | GAAATAGTGATGACGCAGTCTCCAGCCA | |
| | | CCCTGTCTGTGTCTCCAGGGGAAAGAGTC | |
| | | ACCCTCTCCTGCAGGGCCAGTCAGAGTGT | |
| | | TACCAGCGAGTTGGCCTGGTACCAGCAG | |
| | | AAACCTGGCCAGGCTCCCAGGCTCCTCAT | |
| | | CTATGATGCATCCACCGGGGCCACTGGT | |
| | | ATCCCAGCCAGGTTCAGTGGCAGTGGGT | |
| | | CTGGGACAGACTTCACTCTCACCATCAGC | |
| | | AGCCTGCAGTCTGAAGATTTTGCAGTTTA | |
| | | TTACTGTCAGCAGCATAATAACTGGCCTC | |
| | | TCACTTTCGGCGGCGGGACCAAGGTGGA | |
| | | AATCAAAC | |
| KD12-2C10 | Heavy | GAGGTGCAGCTGGTGGAGTCCGGGGGAG | 271 |
| | | GCTTAGTTCAGCCTGGGGGGTCCCTGAG | |
| | | ACTCTCCTGTGCAGCCTCTGGATTCACCT | |
| | | TCAGTAGCTACTGGATGCACTGGGTCCGC | |
| | | CAAGCTCCAGGGAAGGGGCTGGTGTGGG | |
| | | TCTCACGTATTAAGAGTGATGGGAGGAG | |
| | | CATAAGTTACGCGGACTCCGTGAAGGGC | |
| | | CGATTCACCATCTCCAGAGACAACGCCA | |
| | | AGAACACGCTGTATCTGCAAATGAACAG | |
| | | TCTGAGAGCCGAGGACACGGCTGTGTAT | |
| | | TACTGTGCAAGAGATCCCCACGGCACAG | |
| | | CAGCTCCTCCCCGTGATGCTTTTGATATC | |
| | | TGGGGCCAAGGGACAATGGTCACCGTCT | |
| | | C | |
| KD12-2C10L | Light | CAGTCTGCCCTGACTCAGCCTGCCTCCGT | 272 |
| | | GTCTGGGTCTCCTGGACAGTCGATCACCA | |
| | | TCTCCTGCACTGGAACCAGCGGTGATGTT | |
| | | GGGAGTTATAACCTTGTCTCCTGGTACCA | |
| | | ACAGTACCCAGGCAAAGCCCCCAAACTC | |
| | | ATGATTTATGAGGTCGATAAGCGGCCCTC | |
| | | AGGGGTCTCTAATCGCTTCTCTGGCTCCA | |
| | | AGTCTGGCAACACGGCCTCCCTGACAATT | |
| | | TCTGGGCTCCGGGCTGAGGACGAGGCTC | |
| | | ATTATCACTGCTTCTCATATGCAGGTAGT | |
| | | TTGACTTTGGTATTCGGTGGAGGGACCAA | |
| | | GTTGACCGTCCTAG | |
| KD12-1G7L | Light | CAGTCTGTGCTGACTCAGCCACCCTCAGT | 273 |
| | | GTCTGGGACCCCCGGGCAGAGGGTCACC | |
| | | ATCTCTTGTTCTGGAAGCAGCTCCAACAT | |
| | | CGGAAGTAAAACTGCAAACTGGTACCAG | |
| | | AAGCTCCCAGGAACGGCCCCCAAACTCC | |
| | | TCATCTATAGTAACAATCAGCGGCCCTCA | |
| | | GGGGTCCCTGACCGATTCTCTGGCTCCAA | |
| | | GTCAGGCACCTCAGCCTCCCTGGCTATCA | |
| | | GTGGGCTCCAGTCTGAGGATGAGGCTGG | |
| | | GTACTACTGCACAGCATGGGATGACAGC | |
| | | CTGAATGGTCCGGTGTTCGGCGGAGGGA | |
| | | CCAAGCTGACCGTCCTAG | |
| KD12-1G7 | Heavy | GAGGTGCAGCTGGTGGAGTCTGGGGGAG | 274 |
| | | GCTTGGTCCAGCCTGGGGGGTCCCTGAG | |
| | | ACTCTCCTGTGGAGCCTCTGGATTCACCG | |
| | | TCAGTGGCAAGTATATGACCTGGGCCCG | |
| | | CCAGGCTCCTGAGAAGGGACTGGAGTGG | |
| | | GTCTCAGCTATCTATCGCGGTGGTGGCAC | |
| | | ATACTACGCAGACTCCGTGAAGGGCAGA | |
| | | TTCACCATCTCCAGAGACAATTCCAAAA | |
| | | ACATGTTATATCTTCAAATGAACAGCCTG | |
| | | AGAGCCGAGGACACGGCTGTGTATTACT | |
| | | GTGGGGGGTCCGTGATGGTGAGTGCTAC | |
| | | TGACTACTGGGGCCAGGGAACCCTGGTC | |
| | | ACCGTCTC | |
| KD12-1H2K | Light | GACATCCAGATGACCCAGTCTCCATCCTC | 275 |
| | | CCTGTCTGCATCTGTAGGAGACAGAGTC | |
| | | ACCATCACTTGCCGGGCAAGTCAGAGCA | |
| | | TTAGCAGCTATTTAAATTGGTATCAGCAG | |
| | | AAACCAGGGAAAGCCCCTAAGCTCCTGA | |
| | | TCTATGCTGCATCCAGTTTGCAAAGTGGG | |
| | | GTCCCATCAAGGTTCAGTGGCAGTGGAT | |
| | | CTGGGACAGATTTCACTCTCACCATCAGC | |
| | | AGTCTGCAACCTGAAGATTTTGCAACTTA | |
| | | CTACTGTCAACAGAGTTACAGTACCCCGT | |
| | | ACACTTTTGGCCAGGGGACCAAGGTGGA | |
| | | GATCAAAC | |
| KD12-1H2 | Heavy | GAGGTGCAGCTGGTGGAGTCTGGGGGAG | 276 |
| | | GCCTGGTCAAGCCTGGGGGGTCCCTGAG | |
| | | ACTCTCCTGTGCAGCCTCTGGATTCACCT | |
| | | TCAGTAGCTATAGCATGAACTGGGTCCG | |
| | | CCAGGCTCCAGGGAAGGGGCTGGAGTGG | |
| | | GTCTCATCCATTAGTAGTAGTAGTAGTTA | |
| | | CATATACTACGCAGACTCAGTGAAGGGC | |
| | | CGATTCACCATCTCCAGAGACAACGCCA | |
| | | AGAACTCACTGTATCTGCAAATGAACAG | |
| | | CCTGAGAGCCGAGGACACGGCTGTGTAT | |
| | | TACTGTGCGAGAGATCAAATTAGTGGTT | |
| | | ACTGGGGCCAGGGAACCCTGGTCACCGT | |
| | | CTC | |
| KD12-2F2 | Heavy | GAGGTGCAGCTGGTGGAGTCCGGGGGAG | 277 |
| | | GCTTAGTTCAGCCTGGGGGGTCCCTGAG | |
| | | ACTCTCCTGTGTAGCCTCTGGATTCACCT | |
| | | TCAGTAGCTACTGGATGCACTGGGTCCGC | |
| | | CAAGCTCCAGGGAAGGGGCTGGTGTGGG | |
| | | TCTCACGGATTTATCCTGATGGGACTACT | |
| | | ACTGCAAACTACGCGGACTCCGTGAAGG | |
| | | GCCGATTCACCATCTCCAGAGACAACGC | |
| | | CAAGAACACGGTGTATCTGCAAATGAAC | |
| | | AGTCTGAGAGCCGAGGACACGGCTGTTT | |
| | | ATTACTGTGCAAGAGATCTTCGGGAGTCT | |
| | | GATTACTGGGGCCAGGGAACCCTGGTCA | |
| | | CCGTCTC | |
| KD12-2F2L | Light | CAGTCTGCCCTGACTCAGCCTGCCTCCGT | 278 |
| | | GTCTGGGTCTCCTGGACAGTCGATCACCA | |
| | | TCTCCTGCACTGGAACCAGCAGTGACGT | |
| | | GGGTAATTATAATTATGTCTCCTGGCACC | |
| | | AACAACACCCAGGGAAAGCCCCCAAACT | |
| | | CATGATTTATGATGTCAGTAATCGGCCCT | |
| | | CAGGGGTTTCTAATCGCTTCTCTGGCTCC | |
| | | AAGTCTGGCAACACGGCCTCCCTGACCA | |
| | | TCTCTGGGCTCCAGGCTGAGGACGAGGC | |
| | | TGATTATTACTGCAACTCATATACAACCT | |
| | | ACAGCACTCACGTCTTCGGAACTGGGAC | |
| | | CAAGGTCACCGTCCTAG | |

TABLE 2
Antibody amino acid sequences including CDRs

| | | | Entire Sequence | CDR |
|---|---|---|---|---|
| Antibody | Chain | Sequence | SEQ ID NO: | SEQ ID NO: |
| KD7-2H5 | Heavy | EVQLVESGGGLVQPGGSLRLSC | 1 | CDR1: 42 |
| | | AASGFTFSSYWMHWVRQAPG | | CDR2: 74 |
| | | KGLVWVSRINSDGSSTSYADSV | | CDR3: 4 |
| | | KGRFAISRDNAKNTLYLQMNSL | | |
| | | RAEDTAVYYCARALGGWDIDY | | |
| | | WGQGTLVTVS | | |
| KD7-2H5L | Light | QSVLTQPPSASGTPGQRVTISCS | 5 | CDR1: 6 |
| | | GSSSNIGSNYVYWYQQLPGTAP | | CDR2: 7 |
| | | KLLIYRNNQRPSGVPDRFSGSKS | | CDR3: 8 |
| | | GTSASLAISGLRSEDEADYYCA | | |
| | | AWDDSLSGGVFGGGTKLTVLG | | |
| KD10-1C3L | Light | QSVLTQPPSVSAAPGQKVTISCS | 9 | CDR1: 10 |
| | | GGSSSIGNNYVSWYQLLPGTAP | | CDR2: 11 |
| | | KLLIYDNNKRPSGIPDRFSGSKS | | CDR3: 8 |
| | | GTSATLGITGLQTGDEADYYCG | | |
| | | TWDSGLSAGVFGGGTKLTVLG | | |
| KD10-1C3 | Heavy | VQLVESGGGVVQPGRSLRLSCA | 13 | CDR1: 10 |
| | | ASGFTFSDYAVHWVRQAPGKG | | CDR2: 11 |
| | | LEWAALISNDGDFKYYADAVK | | CDR3: 8 |
| | | GRFTISRDNSNNTLYLQMNSLR | | |
| | | TEDTAVYYCARERGRDKVFPP | | |
| | | GYWGQGTLVTVS | | |
| KD7-1B5L | Light | QSVLTQPPSVSAAPGQKVTISCS | 17 | CDR1: 18 |
| | | GSSSNIGNNYVSWYQQLPGTAP | | CDR2: 11 |
| | | KLLIYDNNKRPSGIPDRFSGSKS | | CDR3: 8 |
| | | GTSATLGITGLQTGDEADYYCG | | |
| | | TWDSSLSAEVFGGGTKLTVLG | | |
| KD7-1B5 | Heavy | VQLVESGGGVVQPGRSLRLSCE | 21 | CDR1: 22 |
| | | ASGFTFRSRAMHWVRQAPGKG | | CDR2: 23 |
| | | LGWVAVIWYDGSNKYYADSVK | | CDR3: 8 |
| | | GRFTISRDNSRNTLYLQMNSLR | | |
| | | AEDTAVYYCARDLGSGFSLDY | | |
| | | WGQGTLVTVS | | |
| KD6-1A8L | Light | QAVVTQEPSLTVSPGGTVTLTC | 25 | CDR1: 26 |
| | | ASSTGAVTSAHSPHWFRQKPG | | CDR2: 27 |
| | | QAPRTLIYDTSNKPSWTPARFSG | | CDR3: 8 |
| | | SLLGGKAALTLSGAQPEDEAEY | | |
| | | YCLLSNSGVHFLFGGGTRLTVL | | |
| | | G | | |
| KD6-1A8 | Heavy | EVQLVQSGAEVKKTGSSVKLSC | 29 | CDR1: 30 |
| | | TATGYTFTYRYLHWVRQAPGQ | | CDR2: 31 |
| | | ALEWMGYITIYNGDTNYAQKF | | CDR3: 8 |
| | | QDRVTISRDMSLSTVYMELSSLT | | |
| | | SEDTAMYFCVRSALYGENAFD | | |
| | | FEWGQGTMVTVS | | |
| KD1-1H8 | Heavy | EVQLVESGGGSVQPGGSLRLSC | 33 | CDR1: 78 |
| | | AASGFTFSNYWMHWVRQAPG | | CDR2: 35 |
| | | KGLVWVSRLNIDGSNTFYADSV | | CDR3: 8 |
| | | KGRFTISRDNAKNTLYLQMNSL | | |
| | | RAEDTAVYYCVRGGSDSGDSV | | |
| | | PFDLWGRGTLVTVS | | |
| KD1-1H8K | Light | EIVMTQSPATLSVSPGERATLSC | 37 | CDR1: 38 |
| | | RASQSVSSNLAWYQQNPGQAP | | CDR2: 39 |
| | | RLLIYGASTRATGIPARFSGSGS | | CDR3: 8 |
| | | GTEFTLTISSLQSEDFAVYYCQH | | |
| | | YNNWPFTFGQGTKVEIK | | |
| KD1-2B1 | Heavy | EVQLVESGGGLVQPGGSLRLSC | 41 | CDR1: 42 |
| | | AASGFTFSSYWMHWVRQAPG | | CDR2: 43 |
| | | KGLVWVSRINNDGSSTGYADSV | | CDR3: 8 |
| | | EGRFTISRDNAKNTLYLQINSLR | | |
| | | AEDTAVYYCARQDGHYYYSM | | |
| | | DVWGQGTTVTV | | |
| KD1-2B1L | Light | QSALTQPRSVSGSPGQSVTISCT | 45 | CDR1: 46 |
| | | GTSSDVGGYNYVSWYQQHPGK | | CDR2: 47 |
| | | APKLMIYDVSERPSGVPARFSGS | | CDR3: 8 |
| | | KSGNTASLTISGLQAEDEADYY | | |
| | | CCSYAGNFRWVFGGGTKLTVL | | |
| KD10-1G3 | Heavy | EVQLVESGGGLVQPGGSLKLSC | 49 | CDR1: 50 |
| | | AASGFSFSGAAMHWVRQSSGR | | CDR2: 51 |
| | | GLEWLGRIRSKINDYATAYAES | | CDR3: 8 |
| | | LHGRFTISRDDAKNTAYLQMNR | | |
| | | LKSEDTAIYYCTTVLSKGDHAV | | |
| | | WLGPWGPGTLVTV | | |
| KD10-1G3L | Light | QPVLTQPPSSSASPGESARLTCT | 53 | CDR1: 54 |
| | | LPSDISVAASDIYWYQQKAGSPP | | CDR2: 55 |
| | | NFLLYDPSDSHKGQDSGVPSRF | | CDR3: 8 |
| | | SGSRDGSANSGFLLISGVQSEDE | | |
| | | ADYYCMVWPPNTVGVVFGGG | | |
| | | TTLTV | | |
| KD5-2D1L | Light | QAVVTQEPSLTVSPGGTVTLTC | 57 | CDR1: 58 |
| | | ASSTGAVTSGYYPNWFQQKPG | | CDR2: 59 |
| | | QAPRALIYSTSNKHSWTPARFSG | | CDR3: 8 |
| | | SLLGGKAALTLSGVQPEDEAEY | | |
| | | YCLLFYGGVHVFGAGTKVTVLG | | |
| KD5-2D1 | Heavy | EVQLVESGGDLVQPGGSLRLSC | 61 | CDR1: 62 |
| | | AASGFTFSTYWMHWVRQAPG | | CDR2: 63 |
| | | QGLVWVSRINGNGRITNYADSV | | CDR3: 8 |
| | | KGRFTVSRDNAKNTVDLQMNS | | |
| | | LRAEDTAVYYCARINEWGDV | | |
| | | WGKGTTVTVS | | |
| KD10-1A8K | Light | DIVMTQTPLSLSVTPGQPASISC | 65 | CDR1: 66 |
| | | KSSQSLLHNDGKTFLHWYLQK | | CDR2: 67 |
| | | PGQSPQLLIYEVSSRFSGVPDRFS | | CDR3: 8 |
| | | GSGSGTDFTLKISRVEAEDVGV | | |
| | | YYCMQGIHLPPTFGGGTKVEIKR | | |
| KD10-1A8 | Heavy | EVQLVESGGGLVQPGGSLRLSC | 69 | CDR1: 42 |
| | | AASGFTFSSYWMEWVRQAPGK | | CDR2: 70 |
| | | GLVWVSHISSDGSVTRYVDSVK | | CDR3: 8 |
| | | GRFTISRDNAKNTLYLQMNSLR | | |
| | | AEDTGVYYCAKDLHWNALDV | | |
| | | WGQGTMVTVS | | |
| KD5-2D10K | Light | EIVMTQSPATLSVSPGERATLSC | 72 | CDR1: 38 |
| | | RASQSVSSNLAWYQQKPGQAP | | CDR2: 39 |
| | | RLLIYGASTRATGIPARFSGSGS | | CDR3: 8 |
| | | GTEFTLTISSLQSEDFAVYYCQQ | | |
| | | YNNWPPWTFGQGTKVEIKR | | |
| KD5-2D10 | Heavy | EVQLVESGGGLVQPGGSLRLSC | 42 | CDR1: 42 |
| | | AASGFTFSSYWMHWVRQAPG | | CDR2: 75 |
| | | KGLVWVSRINSDGSSTSYADSV | | CDR3: 8 |
| | | KGRFTISRDNAKNTLYLQMNSL | | |
| | | RAEDTAVYYCARGVRSGWYA | | |
| | | DAFDIWGQGTMVTVS | | |
| KD5-2D7 | Heavy | EVQLVESGGGLVQPGGSLGLSC | 77 | CDR1: 78 |
| | | AASGFTFSNYWMHWVRQAPG | | CDR2: 79 |
| | | KGLVWVSRINSDGSDTSYADSV | | CDR3: 8 |
| | | KGRFTISRDNAENTLYLHMSSL | | |
| | | RAEDTAVYYCARELGYGDYG | | |
| | | MDVWGKGTTVTVS | | |
| KD5-2D7L | Light | QSVLTQPPSASGTPGQRVTISCS | 81 | CDR1: 82 |
| | | GSSSNIGSHSVIWYQQLPGTAPK | | CDR2: 83 |
| | | LLVYSDNQRPSGVPDRFSGSKS | | CDR3: 8 |
| | | GTSASLAISGLQSEDEADYYCA | | |
| | | AWDDSLNHLHVVFGGGTKLTVL | | |
| KD10-2F6 | Heavy | EVQLVESGGGLVQPGGSLRLSC | 85 | CDR1: 86 |
| | | AASGFIFSNYWMHWVRQAPGE | | CDR2: 87 |
| | | GLVWVSRINKDGSSTFYADSVK | | CDR3: 8 |
| | | GRFTISRDNAKNTLYLQMNSLR | | |
| | | AEDTAVYYCTRDFDEWSGYW | | |
| | | GQGTLVTVS | | |
| KD10-2F6L | Light | QSALTQPASVSGSPGQSITISCTG | 89 | CDR1: 90 |
| | | TSSDVGGCEYVSWYQQHPGKV | | CDR2: 67 |
| | | PKLIIYEVSNRPSGVSNRFSGSKS | | CDR3: 8 |
| | | GNTASLTVSGLQAEDEADYFCS | | |
| | | SCTTSGSYVFGAGTKVTVV | | |
| KD11-2E4 | Heavy | EVQLVESGGGVVQPGESLRLSC | 92 | CDR1: 93 |
| | | AASGVTFSSYWMHWVRQVPG | | CDR2: 94 |
| | | KGLVWVARINIDGTSTTYADCV | | CDR3: 8 |
| | | KGRFTISRDNAKNTLYLQMSSL | | |
| | | RAEDTAVYYCATERGLLSGGR | | |
| | | WHSSHFDYWGQGTLVTVS | | |
| KD11-2E4K | Light | DIQMTQSPSTLSASVGDRVTITC | 96 | CDR1: 97 |
| | | RASQSISSWLAWYQQKPGKAP | | CDR2: 98 |
| | | KVLIYKTSSLESGVPS | | CDR3: 8 |
| | | RFSGSGSGTEFTLTISSLQPDDFA | | CDR3: 8 |
| | | TYYCQQYDSSSLTWTFGQGTK | | |
| | | VEIK | | |
| KD11-2E5 | | EVQLVESGGGLVQPGGSLRLSC | 101 | |
| | | AASGFTFSRFWMHWVRQVPG | | |
| | | KGLVWISRINSDATSSSYADSVK | | |
| | | GRFTISRDNAKNTLFLQMNSLR | | |
| | | AEDTAVYYCATSNTALVYLPE | | |
| | | NWGQGTLVTVS | | |
| KD11-2E5K | Light | EIVLTQSPATLSLSPGERATLSCR | 104 | CDR1: 105 |
| | | ASQSVSDYLAWYQQKPGQAPR | | CDR2: 106 |
| | | LLIYDASNRATGIPA | | CDR3: 8 |
| | | RFSGGGSGTDFTLTISSLEPEDFA | | |
| | | VYYCQQRSYWPPTFGQGTKVE | | |
| | | IK | | |
| KD2-1D10L | Light | QSALTQPPSASGSPGQSVTITCT | 108 | CDR1: 46 |
| | | GTSSDVGGYNYVSWYQQHPGK | | CDR2: 67 |
| | | APKLMIYEVSKRPSGVPDRFSGS | | CDR3: 8 |
| | | KSGNTASLTVSGLQAEDEADYH | | |
| | | CSSFAGDNNSPVFGGGTKLTVL | | |
| KD2-1D10 | Heavy | EVQLVESGGGLIHPGGSLRLSCA | 110 | CDR1: 42 |
| | | ASGFTFSSYWMHWVRQAPGK | | CDR2: 111 |
| | | GLVWVSHIKSDGSNTIYADSVK | | CDR3: 8 |
| | | GRFTISRDNAKNTLYLQMNSLR | | |
| | | AEDTAVYYCGRGRSLTPRSAID | | |
| | | YWGQGTLVTV | | |
| KD9-2A4 | Heavy | LVESGGGLVQPGGSLRLSCAAS | 113 | CDR1: 2 |
| | | GFTFSSLAMHWVRQAPGKGLE | | CDR2: 114 |
| | | FASAISSDGGTTYYANSVKGRF | | CDR3: 8 |
| | | TISRDNSKNTLYLQMGSLRDED | | |
| | | MAVYYCARDDLSTSWDLDYW | | |
| | | GQGTLVTV | | |
| KD9-2A4L | Light | QSALTQPASVSGSPGQSITISCTG | 116 | CDR1: 46 |
| | | TSSDVGGYNYVSWYQQHPGKA | | CDR2: 47 |
| | | PKLMIYDVSNRPSGVSNRFSGSK | | CDR3: 8 |
| | | SGNTASLTISGLQAEDEADYYCS | | |
| | | SYTISSTNVFGTGTKVTVL | | |
| KD3-1B6L | Light | QSALTQPASMSGSPGQSITISCT | 118 | CDR1: 119 |
| | | GTSSDVGAHNFISWYQQHPGK | | CDR2: 120 |
| | | APKLMIFEVNKRPSGVSNRFSGS | | CDR3: 8 |
| | | KSGNTASLTISGLQAEDEADYFC | | |
| | | CSYAGYSTWVFGGGTKLTVL | | |
| KD3-1B6 | Heavy | EVQLVESGGGVVQPGGSLRLSC | 122 | CDR1: 123 |
| | | EVSGFIFSSYWMHWVRQVAGK | | CDR2: 75 |
| | | GLVWVSRINSDGSSTSYADSVK | | CDR3: 8 |
| | | GRFTISRDNAKNTLNLQMNFLR | | |
| | | AEDTAVYYCARGGDHGDYGF | | |
| | | FESWGQGTLVTV | | |
| KD3-1C10 | Heavy | EVQLVESGGGLVQPGGSLRLSC | 125 | CDR1: 126 |
| | | AASGFTFSNHWMHWVRQAPG | | CDR2: 127 |
| | | KGLLWVSRIDTGGSTTNYADSV | | CDR3: 8 |
| | | KGRFTISRDSAKNTVYLQMNSL | | |
| | | RAEDTAVYYCARGGLFYYGM | | |
| | | DVWGQGTTVTV | | |
| KD3-1C10K | Light | EIVLTQSPGTLSLSPGERATLSCR | 313 | CDR1: 129 |
| | | ASQTSSSTSLAWYQQKRGQAPR | | CDR2: 39 |
| | | LLIYGASRRATGIP | | CDR3: 8 |
| | | DRFSGSGSGTDFTLTISRLEPEDF | | |
| | | AVYYCQQFSGSPAYTFGQGTK | | |
| | | VEIK | | |
| KD6-2H8K | Light | DIVMTQTPLSLPVTPGEPASISCR | 131 | CDR1: 132 |
| | | SSQSLLDSADGNTYLDWYLQK | | CDR2: 133 |
| | | PGQSPQLLIHTLSHRASGVPDRF | | CDR3: 8 |
| | | SGSGSGTDFTLKISRVEAEDVGV | | |
| | | YYCMQRIAFPVTFGPGTKVDIK | | |
| KD6-2H8 | Heavy | EVQLVESGGGLVQPGGSLRLSC | 135 | CDR1: 78 |
| | | AASGFTFSNYWMHWVRQAPG | | CDR2: 136 |
| | | EGLEWVSRINSDGNKVSYADSV | | CDR3: 8 |
| | | RGRFTISRDNAKNTLYLQMNSL | | |
| | | TGEDTAVYYCARSNWGSADY | | |
| | | WGQGTLVTV | | |
| KD6-1H10 | Heavy | EVQLVESGGGSVQPGGSLRLSC | 138 | CDR1: 139 |
| | | AASGFTFSRYWMHWVRQAPG | | CDR2: 140 |
| | | EGLVWVSRINSDETDKLYADSV | | CDR3: 8 |
| | | KGRFSIFRDNAKNTLYLQMNRL | | |
| | | RAEDTAVYYCARDREDVVVGP | | |
| | | ATQHTIFNSWGQGTLVTV | | |
| KD6-1H10L | Light | QSALTQPASVSGSPGQSITISCTG | 142 | CDR1: 143 |
| | | TSSDVGSSNFVSWYQQHPGKAP | | CDR2: 67 |
| | | KLILYEVSKRPAGVSSRFSGSRS | | CDR3: 8 |
| | | GNTASLTISGAQAEDEADYSCC | | |
| | | STSSVGTLYVFGTGTKVTVL | | |
| KD8-2E9L | Light | QSVLTQPPSVSAAPGQKVTISCS | 145 | CDR1: 146 |
| | | GSNYNIGNNYVSWYQRLPGTA | | CDR2: 11 |
| | | PKLLIYDNNKRPSGIP | | CDR3: 8 |
| | | DRFSGSKSGTSATLGITGLQTGD | | |
| | | EADYYCGTWDSSLRAGVFGGG | | |
| | | TKLTVL | | |
| KD8-2E9 | Heavy | EVQLVESGGGLVQPGGSLRLSC | 148 | CDR1: 149 |
| | | AASGFTFSRHWMHWVRQAPG | | CDR2: 75 |
| | | KGLVWVSRINSDGSSTSNADSV | | CDR3: 8 |
| | | KGRITISRDNGKNTLYLQMNSL | | |
| | | RAEDTAVYYCAREIASGTDAF | | |
| | | DIWGQGTMVTV | | |
| KD9-2H6 | Heavy | EVQLVESGGGLVKPGESLRLSC | 151 | CDR1: 152 |
| | | VGSGFTLTNAWMIWVRQTSGK | | CDR2: 153 |
| | | GLEWVGRIKSKIDGGAIDYGAP | | CDR3: 8 |
| | | VKGRFTISRDDTKNTVYLQMNS | | |
| | | LQTDDTGVYFCTTDRYSTGYY | | |
| | | GMDDYWGQGTLVTV | | |
| KD9-2H6L | Light | SYELTQPPSVSVSPGQTATITCS | 155 | CDR1: 156 |
| | | GDKLGDKYSFWYQQKPGQSPV | | CDR2: 157 |
| | | VVIYQDSKRPSGIPERFSGSNSG | | CDR3: 8 |
| | | NTATLTISGTQAMDEADYYCQV | | |
| | | WDTDSAVFGTGTRVTV | | |
| KD12-1F10K | Light | DIQMTQSPSSLSASVGDRVTITC | 159 | CDR1: 160 |
| | | RASQSISSYLNWYQQKPGKAPK | | CDR2: 161 |
| | | LLIYAASSLQSGVPSRFSGSGSGT | | CDR3: 8 |
| | | DFTLTISSLQPEDFATYYCQQSY | | |
| | | STPYTFGQGTKVEIK | | |
| KD12-1F10 | Heavy | EVQLVESGGGLVKPGGSLRLSC | 163 | CDR1: 164 |
| | | AASGFTFSSYSMNWVRQAPGK | | CDR2: 165 |
| | | GLEWVSSISSSSSYIYYADSVKG | | CDR3: 8 |
| | | RFTISRDNAKNSLYLQMNSLRA | | |
| | | EDTAVYYCARDTISRYWGQGT | | |
| | | LVTV | | |
| KD12-1A10 | Heavy | EVQLVESGGGLVKPGGSLRLSC | 167 | CDR1: 164 |
| | | AASGFTFSSYSMNWVRQAPGK | | CDR2: 168 |
| | | GLEWVSSISGSSSYMHYAESVK | | CDR3: 8 |
| | | GRFTISRDNAKNSLYLQMNSLR | | |
| | | AEDTAVYYCASTGIITYYYASG | | |
| | | VPDYWGQGTLVTV | | |
| KD12-1A10L | Light | QSVLTQPPSASGTPGQRVTMSC | 170 | CDR1: 171 |
| | | SGSSSNIGRNYVYWYQQLPGTA | | CDR2: 7 |
| | | PKLLIYRNNQRPSGVPDRFSGSK | | CDR3: 8 |
| | | SGTSVSLAISGLRSEDEADYYCA | | |
| | | AWDDSLSGVVFGGGTKLTVL | | |
| KD12-2A10K | Light | EIVLTQSPGTLSLSPGERATLSCR | 173 | CDR1: 38 |
| | | ASQSVSSNLAWYQQKPGQAPR | | CDR2: 39 |
| | | VLIYGASSRATGIPDRFSGSRSGT | | CDR3: 8 |
| | | DFTLTISRLEPEDFAVYYCQRYD | | |
| | | NSPRTFGQGTKVEIK | | |
| KD12-2A10 | Heavy | EVQLVESGGGLVKPGGSLRLSC | 175 | CDR1: 176 |
| | | AASGFTFINAWMNWVRQAPG | | CDR2: 177 |
| | | KGLEWVGLIKSKTDGGTIDYAA | | CDR3: 8 |
| | | PVKGRFTISRDDSEKMLYLQMD | | |
| | | SLKTEDTAVYYCTTMYGWKD | | |
| | | ARDYWGQGTLVTV | | |
| KD12-2A1K | Light | EIVLTQSPGTLSLSPGERATLSCR | 179 | CDR1: 180 |
| | | ASQSVSSSYLAWYQQKPGQAPR | | CDR2: 39 |
| | | FLIYGASSRATGIPDRFSGSGSGT | | CDR3: 8 |
| | | DFTLTISRLEPEDFAVYYCQQY | | |
| | | GSSPYTFGQGTKVEIK | | |
| KD12-2A1 | Heavy | EVQLVESGGGLVQPGGSLRLSC | 182 | CDR1: 42 |
| | | AASGFTFSSYWMHWVRQAPG | | CDR2: 183 |
| | | KGLVWVSRIKSDGSSTSYADSV | | CDR3: 8 |
| | | KGRFTISRDNAKNTLYLQMNSL | | |
| | | RAEDTAVYYCARGGGSSNWYP | | |
| | | GFFDYWGQGTLVTV | | |
| KD12-2H1 | Heavy | EVQLLESGGGLVQPGGSLRLSC | 185 | CDR1: 286 |
| | | VASGFTFSSYAMSWVRQAPGK | | CDR2: 187 |
| | | GLEWVSSISASGGSTYYADSVK | | CDR3: 8 |
| | | GRFTISRDNSKNTLYLQMNSLR | | |
| | | AEDTAVYYCGNWPEGFPAYFH | | |
| | | YWGQGTLVTV | | |
| KD12-2H1K | Light | EIVMTQSPATLSVSPGERVTLSC | 189 | CDR1: 190 |
| | | RASQSVTSELAWYQQKPGQAP | | CDR2: 106 |
| | | RLLIYDASTGATGIPARFSGSGS | | CDR3: 8 |
| | | GTDFTLTISSLQSEDFAVYYCQQ | | |
| | | HNNWPLTFGGGTKVEIK | | |
| KD12-2C10 | Heavy | EVQLVESGGGLVQPGGSLRLSC | 192 | CDR1: 42 |
| | | AASGFTFSSYWMHWVRQAPG | | CDR2: 193 |
| | | KGLVWVSRIKSDGRSISYADSV | | CDR3: 8 |
| | | KGRFTISRDNAKNTLYLQMNSL | | |
| | | RAEDTAVYYCARDPHGTAAPP | | |
| | | RDAFDIWGQGTMV TV | | |
| KD12-2C10L | Light | QSALTQPASVSGSPGQSITISCTG | 196 | CDR1: 197 |
| | | TSGDVGSYNLVSWYQQYPGKA | | CDR2: 198 |
| | | PKLMIYEVDKRPSGVSNRFSGS | | CDR3: 8 |
| | | KSGNTASLTISGLRAEDEAHYH | | |
| | | CESYAGSLTLVFGGGTKLTVL | | |
| KD12-1G7L | Light | QSVLTQPPSVSGTPGQRVTISCS | 200 | CDR1: 201 |
| | | GSSSNIGSKTANWYQKLPGTAP | | CDR2: 202 |
| | | KLLIYSNNQRPSGVPDRFSGSKS | | CDR3: 8 |
| | | GTSASLAISGLQSEDEAGYYCT | | |
| | | AWDDSLNGPVFGGGTKLTVL | | |
| KD12-1G7 | Heavy | EVQLVESGGGLVQPGGSLRLSC | 204 | CDR1: 205 |
| | | GASGFTVSGKYMTWARQAPEK | | CDR2: 206 |
| | | GLEWVSAIYRGGGTYYADSVK | | CDR3: 8 |
| | | GRFTISRDNSKNMLYLQMNSLR | | |
| | | AEDTAVYYCGGSVMVSATDY | | |
| | | WGQGTLVTV | | |
| KD12-1H2K | Light | DIQMTQSPSSLSASVGDRVTITC | 159 | CDR1: 160 |
| | | RASQSISSYLNWYQQKPGKAPK | | CDR2: 208 |
| | | LLIYAASSLQSGVPSRFSGSGSGT | | CDR3: 8 |
| | | DFTLTISSLQPEDFATYYCQQSY | | |
| | | STPYTFGQGTKVEIK | | |
| KD12-1H2 | Heavy | EVQLVESGGGLVKPGGSLRLSC | 209 | CDR1: 164 |
| | | AASGFTFSSYSMNWVRQAPGK | | CDR2: 165 |
| | | GLEWVSSISSSSSYIYYADSVKG | | CDR3: 8 |
| | | RFTISRDNAKNSLYLQMNSLRA | | |
| | | EDTAVYYCARDQISGYWGQGT | | |
| | | LVTV | | |
| KD12-2F2 | Heavy | EVQLVESGGGLVQPGGSLRLSC | 211 | CDR1: 42 |
| | | VASGFTFSSYWMHWVRQAPG | | CDR2: 212 |
| | | KGLVWVSRIYPDGTTTANYADS | | CDR3: 8 |
| | | VKGRFTISRDNAKNTVYLQMNS | | |
| | | LRAEDTAVYYCARDLRESDYW | | |
| | | GQGTLVTV | | |
| KD12-2F2L | Light | QSALTQPASVSGSPGQSITISCTG | 214 | CDR1: 215 |
| | | TSSDVGNYNYVSWHQQHPGKA | | CDR2: 47 |
| | | PKLMIYDVSNRPSGVSNRFSGSK | | CDR3: 8 |
| | | SGNTASLTISGLQAEDEADYYC | | |
| | | NSYTTYSTHVFGTGTKVTVL | | |

In an aspect, provided herein are nucleic acids or polynucleotides encoding antibodies or antigen binding fragments described herein. In some aspects, the antibodies or fragments comprise the sequences in Table 1 or Table 2, or a sequence having at least 90% or 95% sequence identity to the sequences in Table 1 or Table 2.

In some aspects the ICI or KD antibody or antigen binding fragment thereof comprises an antibody capable of binding to a epitope. In some aspects the epitope comprises P⁸, L¹⁰, Q¹², S¹³ and I¹⁴, in some aspects the epitope may comprise M⁹ and V¹⁵, in some aspects the epitope may comprise F¹¹, wherein the capital letter is the amino acid, and the superscript number is the position relative to SEQ ID NO: 281 or KDpeptide3. In some aspects the epitope comprises SEQ ID NO: 280 or KDpeptide2, in some aspects the epitope comprises SEQ ID NO: 279 or KDpeptide1, in some aspects the epitope comprises SEQ ID NO: 281. In some aspects the epitope comprises the amino acid sequence of PMLF(V,T)QSIV of SEQ ID NO: 282, or SEQ ID NO: 283 (PMLFQSIV) or sequences 90% identical thereof.

Protein] and nucleic acid sequence identities are evaluated using the Basic Local Alignment Search Tool (“BLAST”) which is well known in the art (Karlin and Altschul, 1990, Proc. Natl. Acad. Sci. USA 87:2267-2268; Altschul et al., 1997, Nucl. Acids Res. 25:3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 85% sequence identity to the SEQ ID. Alternatively, percent identity can be any integer from 85% to 100%. More preferred embodiments include at least: 85%, 86%, 87%, 88%, 89%, 90%, 91%, 92%, 93%, 94%, 95%, 96%, 97%, 98% or 99% compared to a reference sequence using the programs described herein, preferably BLAST using standard parameters, as described. These values can be appropriately adjusted to determine corresponding identity of proteins encoded by two nucleotide sequences by taking into account codon degeneracy, amino acid similarity, reading frame positioning, and the like.

“Substantial identity” of amino acid sequences for purposes of this invention normally means polypeptide sequence identity of at least 85%. Preferred percent identity of polypeptides can be any integer from 85% to 100%. More preferred embodiments include at least 85%, 86%, 87%, 88%, 89%, 90%, 91%, 92%, 93%, 94%, 95%, 96%, 97%, 98%, or 99%.

The antibody may be linked to a tag, detectable label or additional moiety. The isolated antibody or fragment thereof is directly or indirectly linked to a tag or detectable label. The antibody or fragment thereof may be conjugated to the tag or detectable label. The tag or detectable label is a polypeptide, wherein the polypeptide is translated concurrently with the antibody polypeptide sequence.

The term “tag” or “detectable label” as used herein includes any useful moiety that allows for the purification, identification, detection, diagnosing, imaging, or therapeutic use of the antibody of the present invention and are readily known in the art. Suitable tags or detection labels include epitope tags, detection markers and/or imaging moieties, including, for example, enzymatic markers, fluorescence markers, radioactive markers, among others.

The term “additional moiety” includes other molecules that may be linked to the antibody or antibody fragment thereof. Suitable additional moieties include, but are not limited to, for example, therapeutic agents, small molecules, and drugs, among others. The additional moieties can also include diagnostic agents.

The detectable label may be a biotin or a biotinylated tag. The detectable label may be a fluorescent protein, luciferase, a fluorescent compound, or a colorimetric reagent.

In an aspect, provided herein is a nucleic acid or polynucleotide encoding an antibody or antibody fragment described herein. The polynucleotide may be a polynucleotide construct encoding the polypeptide, antibody or antibody fragment described herein. The nucleic acid construct may be an expression vector or vector capable of expressing the protein or polynucleotide in a host cell.

The present invention also provides expression vectors comprising a polynucleotide encoding the antibodies or fragments of the present invention. Advantageously, the expression vector is a recombinant expression vector comprising an “expression cassette” or an “expression construct” according to the present invention. Within the construct, the polynucleotide may operatively linked to a transcriptional promoter (e.g., a heterologous promoter) allowing the construct to direct the transcription of said polynucleotide in a host cell. Such vectors are referred to herein as “recombinant constructs,” “expression constructs,” “recombinant expression vectors” (or simply, “expression vectors” or “vectors”).

Suitable vectors are known in the art and contain the necessary elements in order for the gene encoded within the vector to be expressed as a protein in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments encoding the antibodies or fragments thereof. Another type of vector is a viral vector, wherein additional DNA segments may be ligated into the viral genome to be expressed in viral particles to be infected into cells and allow expression of the viral vectors carried within the viral particles.

Certain vectors are capable of autonomous replication in a host cell into which they are introduced. Other vectors can be integrated into the genome of a host cell upon introduction into the host cell, and thereby are replicated along with the host genome (e.g. lentiviral vectors). Viral vectors include those known in the art, e.g., replication defective retroviruses (including lentiviruses), adenoviruses and adeno-associated viruses (rAAV)), which serve equivalent functions. Lentiviral vectors may be used to make suitable lentiviral vector particles by methods known in the art to transform cells in order to express the antibody or antigen binding fragment thereof described herein.

The present invention also provides a host cell comprising the isolated nucleic acids or expression vectors described herein that are capable of producing the antibodies or antibody fragments thereof. In one embodiment, the host cell is a hybridoma cell. In another embodiment, the host cell contains a recombinant expression cassette or a recombinant expression vector according to the present invention and is able to express the encoded antibody or antigen binding fragment thereof. The host cell can be a prokaryotic or eukaryotic host cell. Suitable host cells include, but are not limited to, mammalian cells, bacterial cells and yeast cells. In some embodiments, the host cell may be a eukaryotic cell. The term “host cell” includes a cell into which exogenous nucleic acid has been introduced, including the progeny of such cells. Host cells also include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity that was screened or selected for in the originally transformed cell are included herein. It should be appreciated that the host cell can be any cell capable of expressing antibodies, for example fungi; mammalian cells; insect cells, using, for example, a baculovirus expression system; plant cells, such as, for example, corn, rice, Arabidopsis, and the like. See, generally, Verma, R. et al., J Immunol Methods. 1998 Jul. 1; 216 (1-2): 165-81. Host cell also include hybridomas that produce the monoclonal antibodies described herein.

### Peptides Specific for Kawasaki Disease

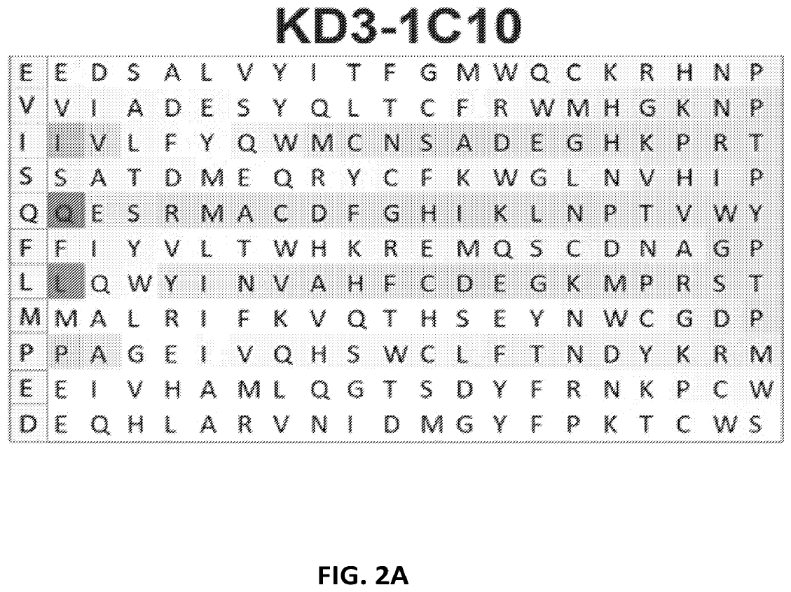

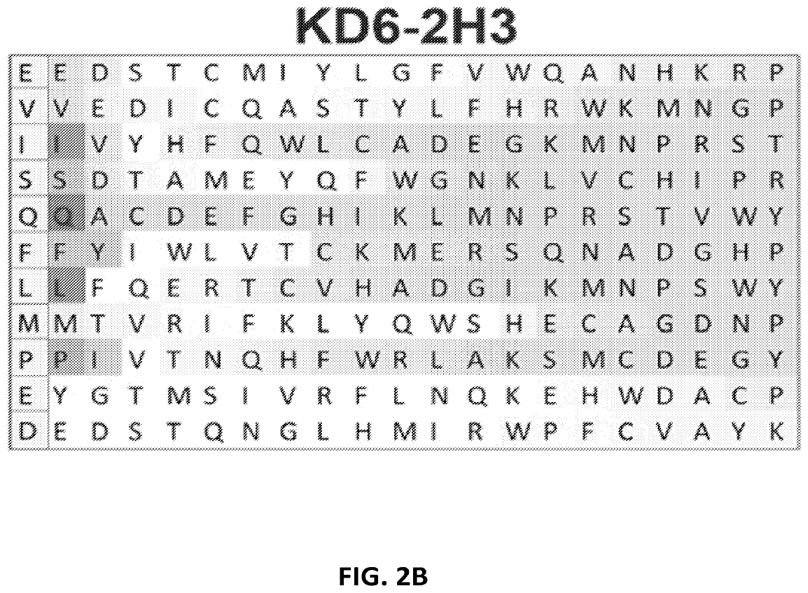

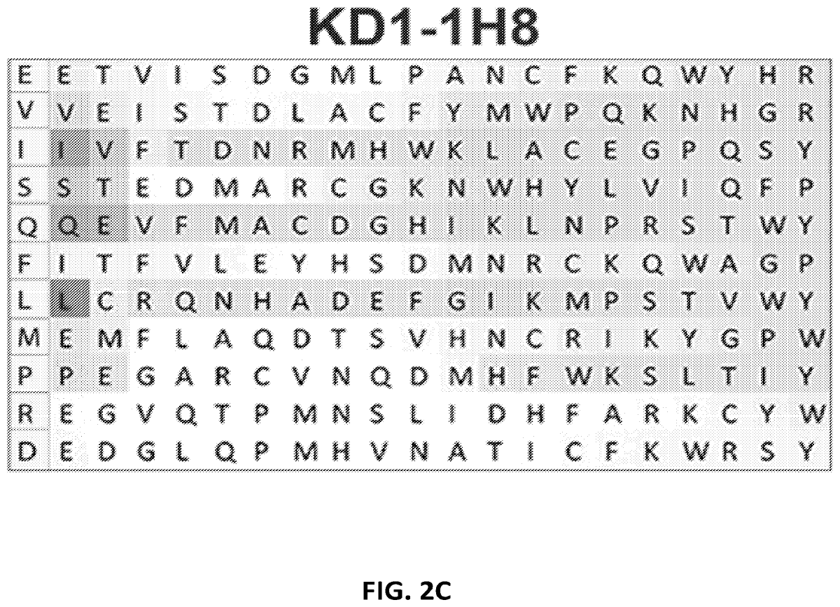

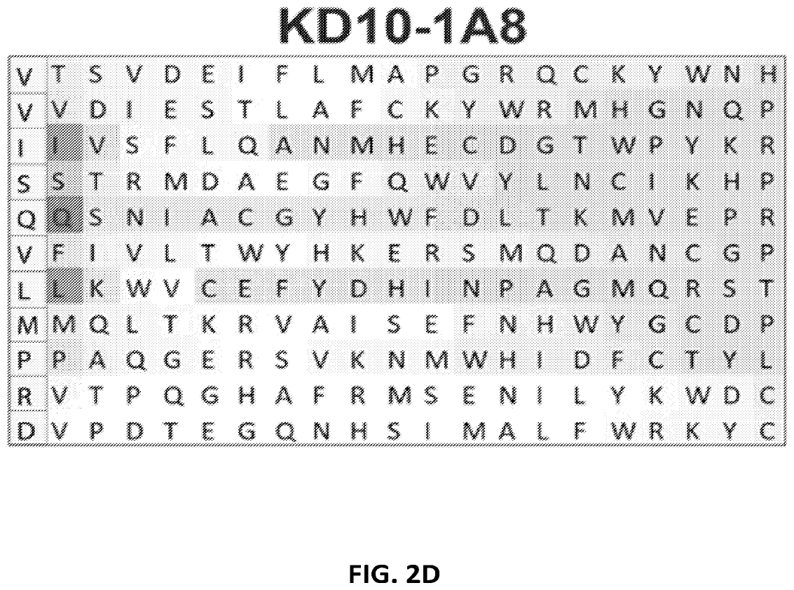

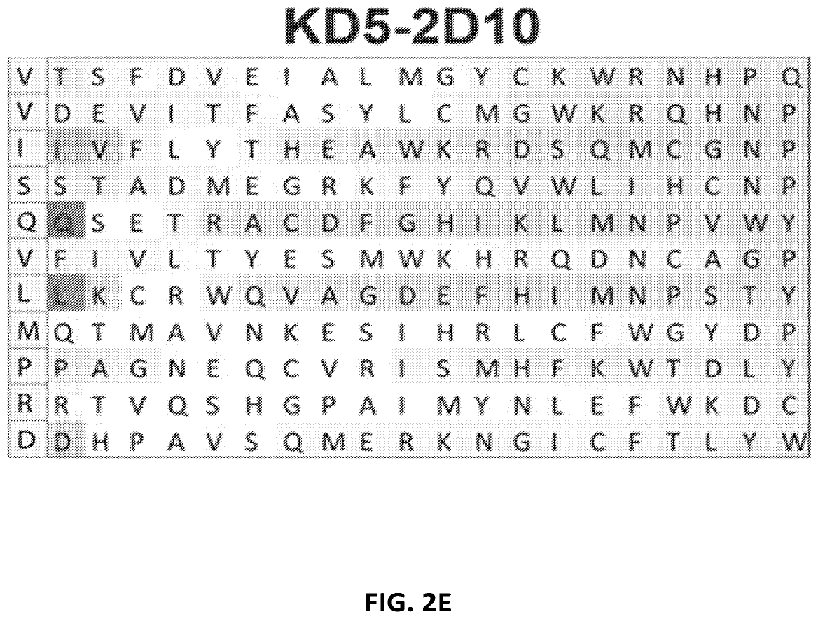

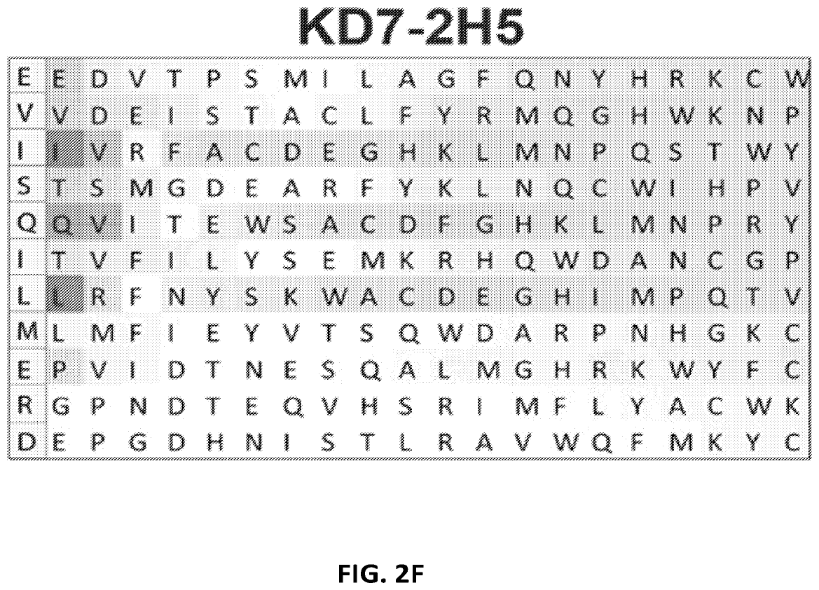

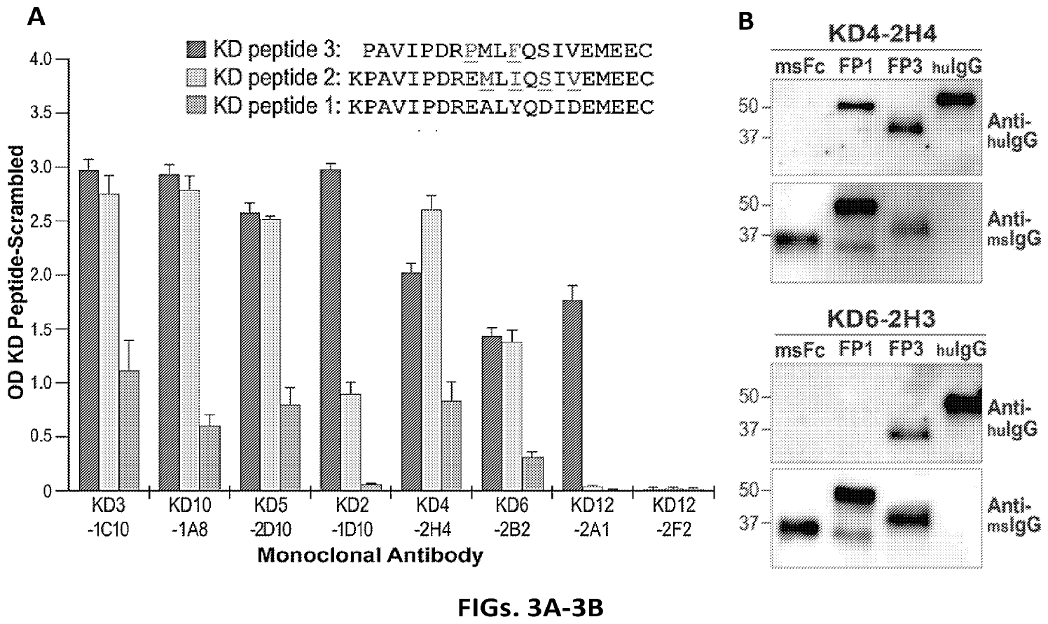

The inventors discovered that patients diagnosed with Kawasaki disease produce antibodies that recognize a particular peptide (see FIGS. 2 and 3). Accordingly, in another aspect of the current disclosure, peptides specific for binding antibodies found in subjects with Kawasaki disease are provided. The peptides comprise SEQ ID NO: 281 (KD3) or a sequence with 95% similarity to SEQ ID NO: 281. The peptides may comprise a detectable label or tag. The peptides may be linked to a solid support. The peptides may be used in a method of detecting the presence of antibodies associated with Kawasaki diseases. A polynucleotide construct capable of expressing the polypeptide is also provided.

As used herein, “solid support” refers to any suitable material for the immobilization peptides (including, but not limited to epitope fragments, antibodies, or antibody fragments thereof) of the instant disclosure. For example, the solid support may be beads, particles, tubes, wells, probes, dipsticks, pipette tips, slides, fibers, membranes, papers, natural and modified celluloses, polyacrylamides, agaroses, glass, polypropylene, polyethylene, polystyrene, dextran, nylon, amylases, plastics, magnetite or any other suitable material readily known to one of skill in the art.

### Methods of Detection, Imaging and Diagnosing Kawasaki Disease

In an aspect, methods of detecting antibodies associated with Kawasaki disease and treating KD are provided using the polypeptide KD3 are described herein

The peptide KD3 (SEQ ID NO: 281) or a fragment having at least 95% sequence identity, more particularly at least 98% sequence identity, is used as a detection agent for detecting the presence of antibodies associated with Kawasaki Disease (KD) in a sample. For example, the method of detecting antibodies associated with Kawasaki disease in a subject may comprise the steps of: i) obtaining a sample from a subject suspected of having Kawasaki disease; ii) contacting the sample with the peptide described here in (e.g., KD3, i.e., SEQ ID NO: 281 or a polypeptide having at least 95% sequence similarity to SEQ ID NO: 281); and iii) detecting the specific binding of antibodies to the peptide to form a peptide-antibody complex, wherein the presence of a peptide-antibody complex confirms the presence of antibodies associated with Kawasaki disease in the subject. Methods of detection of the peptide-antibody complex include methods known in the art, for example, enzyme-linked immunoabsorbent assay (ELISA), Western blot, immunostaining, immunoprecipitation, flow cytometry, sensor chips, magnetic beads, nanoparticles, and the like. If antibodies are detected within the sample, the method can further comprise: iv) treating the subject having antibodies associated with Kawasaki disease with intravenous immunoglobulin (IV Ig). Other treatment options for KD could also be employed and considered within the scope of this invention.

The methods may be carried out using a kit comprising the peptide. The peptide may be linked to a solid support. The peptide may further comprise or be linked to a detectable label.

The detecting of the peptide-antibody complex may comprise contacting the peptide-antibody complex with a secondary antibody wherein the secondary antibody is optionally linked to a detectable label. Suitable secondary antibodies include, but are not limited to anti-human antibodies that specifically bind to human Fc region of antibodies, particularly to IgG antibodies. Other suitable anti-human antibodies that could specifically recognize human antibodies bound to the peptide are also contemplated.

The methods may comprise the steps of i) obtaining a sample from a subject suspected of having Kawasaki Disease; ii) contacting the sample with an antibody or antigen binding fragment thereof of Table 2; iii) detecting the binding of the antibody to a component of the sample, whereby binding of the antibody to the component of the sample indicates the presence of an antigen associated with Kawasaki Disease. The detection can confirm the diagnosis of Kawasaki disease in the subject. As used herein, “component of the sample” refers to any molecule present in a subject's sample which is capable of being bound by an Kawasaki-specific antibodies described herein, for example, proteins, peptides, viral particles, carbohydrates, glycoproteins, and the like, that is specific for the Kawasaki disease associated antibody and does not bind to a control antibody.

The term “contacting” or “exposing,” as used herein refers to bringing a disclosed antibody and a cell, a target receptor, a biological sample, or other biological entity, together in such a manner that the antibody can detect and/or affect the activity of the target, either directly; i.e., by interacting with the target itself, or indirectly; i.e., by interacting with another molecule, co-factor, factor, or protein that is attached to said target.

The methods may further comprise iv) treating the subject having a component associated with Kawasaki disease (and in some cases, diagnosed with Kawasaki disease) with intravenous administrated immunoglobulin (IV Ig). As used herein, the terms “treating” or “to treat” each mean to alleviate symptoms, eliminate the causation of resultant symptoms either on a temporary or permanent basis, and/or to prevent or slow the appearance or to reverse the progression or severity of symptoms of Kawasaki disease. As used herein, “intravenous Ig” refers to an effective amount of pooled IgG from donor subjects. Trade names of intravenous immunoglobulin formulations include Flebogamma®, Gamunex®, Privigen®, Octagam®, and Gammagard®, while trade names of subcutaneous formulations include Cutaquig®, Cuvitru®, HyQvia®, Hizentra®, Gamunex-C®, and Gammaked®.

The “sample” or “biological sample” for the detection methods described herein are any biological sample obtained from the patient or subject that comprises antibodies. The sample may be a blood sample, a tissue sample, or a serum sample.

Detecting the binding of the antibody or antigen binding fragment thereof in the sample may be carried out using ELISA, Western blot, immunostaining, immunoprecipitation, flow cytometry, sensor chips, magnetic beads, nanoparticles and the like

Patients with KD have intracytoplasmic inclusion bodies present in some tissues, e.g., respiratory epithelium. Therefore, in another aspect of the current disclosure, methods of detecting intracytoplasmic inclusion bodies in a subject are provided. The methods may comprise the steps of: i) obtaining a sample from a subject suspected of having Kawasaki Disease; ii) contacting the sample with an antibody or antigen binding fragment thereof of Table 2; and iii) detecting the binding of the antibody or antigen binding fragment thereof in the sample, whereby binding of the antibody indicates the presence of intracytoplasmic inclusion bodies. The methods may further comprise iv) treating the subject having ICI bodies with intravenous immunoglobulin (IV Ig).

Suitable methods of detection are known in the art and include, but are not limited to, for example, ELISA, Western blot, immunostaining, immunoprecipitation, flow cytometry, sensor chips, magnetic beads, and the like.

In another aspect of the current disclosure, further methods of diagnosing Kawasaki disease are provided. The methods may comprise the steps of: i) obtaining a sample comprising antibodies from a subject suspected of having Kawasaki disease; ii) contacting the sample with a peptide comprising SEQ ID NO: 281, or a peptide comprising a sequence with 95% similarity to SEQ ID NO: 281; and iii) detecting the binding of antibodies to the peptide to form an peptide-antibody complex, wherein the presence of a peptide-antibody complex confirms the diagnosis of Kawasaki disease in the subject. The methods may further comprise iv) treating the subject diagnosed with Kawasaki disease with intravenous immunoglobulin (IV Ig). The peptide may be linked to a solid support. Detecting may comprise contacting the peptide-antibody complex with a secondary antibody wherein the secondary antibody is optionally linked to a detectable label.

### Kits for Diagnosing Kawasaki Disease and Detecting Intracytoplasmic Inclusion Bodies

In another aspect of the current disclosure, kits are provided. Any suitable kits comprising the components to carry out the methods described herein are contemplated.

The kits may comprise: i) an antibody or antigen binding fragment thereof of Table 2; and ii) a detection reagent. The kits may further comprise: iii) a solid support. The antibody or antigen binding fragment thereof may be linked to the solid support. The solid support may comprise a lateral flow device. The solid support may be the inner, bottom surface of a well of a microtiter plate or a substrate that is included as part of a lateral flow device, for example. The reagents employed in the methods of using the kit may be dried or immobilized onto the solid support, which may comprise a chromatographic support, contained within the device.

An exemplary lateral flow device is the lateral flow device that is described in U.S. Pat. No. 5,726,010, which is incorporated herein by reference in its entirety. The device for performing a lateral flow assay may be a SNAP® device, which is commercially available from IDEXX Laboratories, Inc. of Westbrook, Me. However, it is to be understood that the skilled artisan will recognize that a large variety of other lateral flow devices that are not SNAP® devices or described by U.S. Pat. No. 5,726,010 allow for the immobilization of an antibody thereon, and therefore would be suitable for being used in the methods and kits device of the present invention.

Peptide and antibodies used in the methods and kits of the invention may be immobilized on the solid support by any methodology known in the art, including, for example, covalently or non-covalently, directly or indirectly, attaching the antibodies to the solid support. Therefore, while these antibodies may be attached to the solid support by physical adsorption (i.e., without the use of chemical linkers), it is also true that these antibodies may be immobilized to the solid support by any chemical binding (i.e., with the use of chemical linkers) method readily known to one of skill in the art.

In some embodiments, the kits may comprise: i) a peptide comprising SEQ ID NO: 281, or a peptide comprising a sequence with 95% similarity to SEQ ID NO: 281; and ii) a detection reagent. The kits may comprise iii) a solid support. The detection reagent may comprise a secondary antibody optionally linked to a detectable agent or label. The detection agent may be linked to the polypeptide. The kit may include instructions. The polypeptide may be linked to a solid support. The solid support may be a lateral flow device. The kit may further comprise an anti-human Fc antibody capable of detecting human antibodies.

A suitable kit may be an ELISA kit capable of detecting the binding the peptide to a human antibody, and therefore the kit may further comprise an antibody capable of binding the Fc portion of human antibodies.

The present invention has been described in terms of one or more preferred embodiments, and it should be appreciated that many equivalents, alternatives, variations, and modifications, aside from those expressly stated, are possible and within the scope of the invention.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

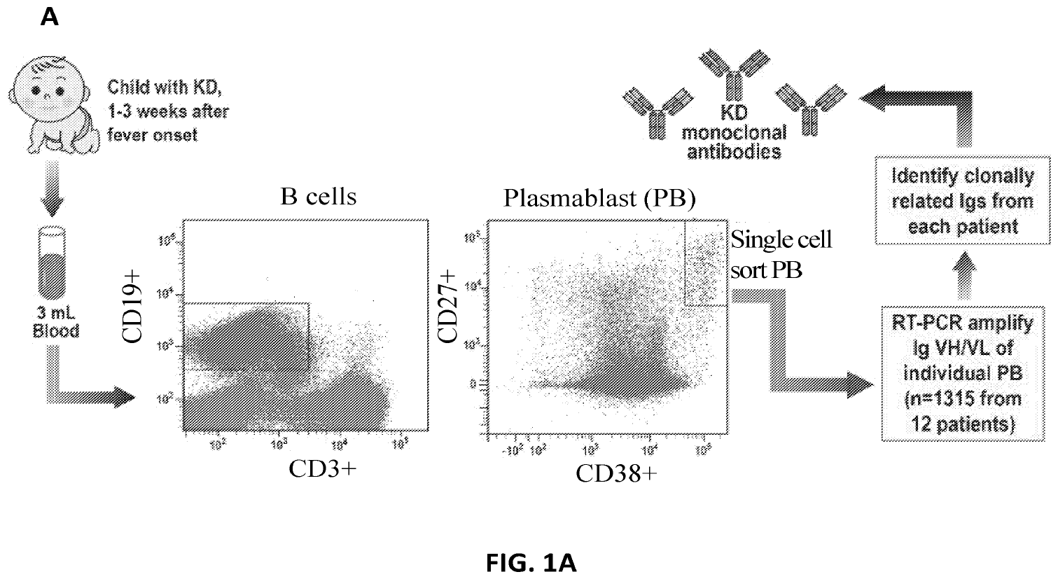

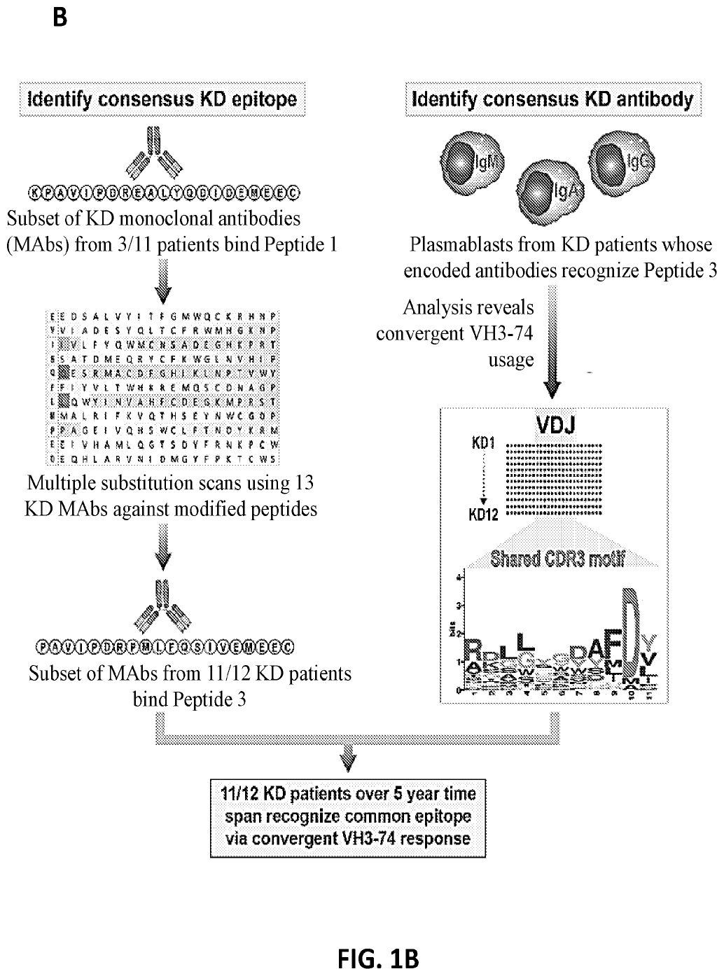

To characterize the antibody response in KD patients, the inventors generated MAbs from clonally expanded peripheral blood plasmablasts obtained by single cell sorting from 12 children with KD (FIG. 1A, Table 3). In prior work, the inventors reported that KD MAbs from 3 patients, designated KD4-2H4, KD6-2B2, and KD8-1D4, bind to intracytoplasmic inclusion bodies in tissues from fatal KD cases and recognize a specific epitope designated as KDpeptide1. These MAbs did not recognize a human protein by protein array analyses 17. The MAbs are described in PCT Application No. PCT/US2020/020440 which is incorporated herein by reference in its entirety. In the present disclosure, the inventors prepared additional KD MAbs from a total of 12 KD patients to determine whether the specific epitope could be refined to optimize binding to KD MAbs. The inventors also analyzed the KD MAbs that bound to the refined epitope, to determine if they shared sequence similarity between KD patients (FIG. 1B).

TABLE 3
Clinical and plasmablast data for Kawasaki disease patients in this study¹

| | | | | | Day | Day | | | |
|---|---|---|---|---|---|---|---|---|---|
| | | | | | of | of | | | Total |
| | | | | | illness | illness | | Zmax | # |
| | | | | Race/ | of | IVIG | Incomplete | RCA/ | Plasmablasts |
| Patient | Age | | Gender | Ethnicity | sample | given | KD | LAD | (isotype) |
| KD1 | 4 | yr | F | Hispanic | 17 | 13 | No | RCA 4.7 | 119 |
| | | | | | | | | | (49A, 28G, 42M) |
| KD2 | 4 | yr | F | Caucasian | 24 | 8 | No | NL | 115 |
| | | | | | | | | | (64A, 22G, 29M) |
| KD3 | 5 | yr | M | Black | 20 | 19 | Yes | NL | 67 (33A, 31G, 3M) |
| KD4 | 3 | mo | M | Hispanic | 8 | 6 | Yes | RCA 3.0, | 153 |
| | | | | | | | | LAD | (102G, 30A, 21M) |
| | | | | | | | | 3.5 | |
| KD5 | 11 | mo | F | ½ Caucasian, | 14 | 10 | Yes | NL | 115 |
| | | | | ½ Asian | | | | | (63G, 39A, 13M) |
| | | | | (Vietnamese) | | | | | |
| KD6 | 4 | yr | F | Black | 13 | 5 and 8 | No | RCA 3.0 | 80 (43A, 23G, |
| | | | | | | | | | 14M) |
| KD7 | 17 | mo | M | Caucasian | 13 | 9 | No | RCA 9.8, | 121 |
| | | | | | | | | LAD | (56A, 50G, 15M) |
| | | | | | | | | 5.7 | |
| KD8 | 17 | mo | M | Caucasian | 14 | 12 | Yes | LAD 3.5 | 85 |
| | | | | | | | | | (41A, 34G, 10M) |
| KD9 | 3 | yr | M | Asian (Indian) | 9 | 6 | No | NL | 81 |
| | | | | | | | | | (30A, 38G, 13M) |
| KD10 | 5 | yr | F | Asian (½ Japanese, | 11 | 7 | No | RCA 5.4, | 88 (41A, 41G, 6M) |
| | | | | ½ Korean) | | | | LAD | |
| | | | | | | | | 7.4, distal | |
| | | | | | | | | large R, L | |
| | | | | | | | | CAA | |
| KD11 | 3 | yr | F | Caucasian | 15 | 5 | No | RCA 2.55, | 132 (36A, 50G, |
| | | | | | | | | distal L | 46M) |
| | | | | | | | | main 5.6 | |
| KD12 | 7 | mo | M | Caucasian | 22 | 18 | No | RCA 4.3 | 159 (69A, 34G, |
| | | | | | | | | LAD 11.6 | 56M) |

Zmax = maximal Z score;
RCA = right coronary artery,
LAD = left anterior descending coronary artery,
IVIG = intravenous gammaglobulin
¹Patients 1-11 previously reported in Rowley A H et al., J Infect Dis 222, 158-168 (2020)

Sequencing analysis of MAbs recognizing KDpeptide1 shows use of the VH3-74 family and its paralogs. KD4-2H4, KD6-2B2, and KD8-1D4, the MAbs originally identified as binding to KDpeptide1, are members of the VH3-74, VH3-33, and VH3-72 families, which are paralog VH families with similarity scores of ≥82% 21. To determine if plasmablasts encoding antibodies from the VH3-74 family preferentially bind to the epitope, in this study the inventors prepared additional MAbs from eight additional KD patients. This yielded VH3-74 antibodies that recognized the peptide epitope by ELISA from KD3 (KD3-1C10), KD5 (KD5-2D7 and KD5-2D10), and KD10 (KD10-1A8) (Table 4, VH3-74 MAbs bolded, Table 5). These results suggest that VH3-74 antibodies and their paralogs might be preferentially used by children with KD to respond to the protein epitope, presumably because the antibody structure of this family allows for binding to this KD antigen.

TABLE 4
MAbs generated from plasmablasts of patients with KD that bind to KD peptide

| | | | | | IGHV AA | | | IGK(L)V | |
|---|---|---|---|---|---|---|---|---|---|
| | | | | HCDR3 | mutations from | | K(L) | AA | PB |
| Patient | MAb | IGHV | JH | length | GL | IGK(L)V | CDR3 | mutations | Isotype |
| KD4 | KD4-2H4 | 3- | 2 | 14 | 4 | L2-11*01 | 10 | 0 | IgG |
| | | 74*01 | | | | | | | |
| KD6 | KD6-2B2 | 3- | 3 | 12 | 15 | K1-5*03 | 10 | 9 | IgA |
| | | 33*01 | | | | | | | |
| KD8 | KD8-1D4 | 3- | 3 | 12 | 1 | K1-6*01 | 9 | 4 | IgA |
| | | 72*01 | | | | | | | |
| KD1 | KD1-2B1 | 3- | 6 | 13 | 4 | L2-11*01 | 10 | 3 | IgA |
| | | 74*01 | | | | | | | |
| KD1 | KD1-1H8 | 3- | 2 | 15 | 7 | K3- | 9 | 2 | IgA |
| | | 74*01 | | | | 15*01 | | | |
| KD2 | KD2-1D10 | 3- | 4 | 14 | 7 | L2-8*01 | 11 | 4 | IgG |
| | | 74*01 | | | | | | | |
| KD3 | KD3-1C10 | 3- | 6 | 12 | 10 | K3- | 10 | 9 | IgA |
| | | 74*01 | | | | 20*01 | | | |
| KD5 | KD5-2D7 | 3- | 6 | 13 | 6 | L1-44*01 | 13 | 6 | IgG |
| | | 74*01 | | | | | | | |
| KD5 | KD5-2D10 | 3- | 3 | 15 | 0 | K3- | 10 | 0 | IgG |
| | | 74*01 | | | | 15*01 | | | |
| KD6 | KD6-2H3 | 3- | 3 | 19 | 9 | K3- | 8 | 5 | IgG |
| | | 15*01 | | | | 15*01 | | | |
| KD7 | KD7-2H5 | 3- | 4 | 11 | 1 | L1-47*01 | 11 | 0 | IgA |
| | | 74*01 | | | | | | | |
| KD7 | KD7-1B5 | 3- | 4 | 12 | 6 | L1-51*01 | 11 | 0 | IgA |
| | | 33*01 | | | | | | | |
| KD10 | KD10-1G3 | 3- | 5 | 15 | 14 | L5-37*01 | 11 | 20 | IgA |
| | | 73*01 | | | | | | | |
| KD10 | KD10-1A8 | 3- | 3 | 11 | 8 | K2- | 9 | 3 | IgG |
| | | 74*01 | | | | 29*02 | | | |
| KD11 | KD11-2E4 | 3- | 4 | 19 | 11 | K1-5*03 | 11 | 4 | IgA |
| | | 74*01 | | | | | | | |
| KD11 | KD11- | 3- | 4 | 13 | 0 | K3- | 10 | 0 | IgA |
| | 2A12 | 21*01 | | | | 20*01 | | | |
| KD12 | KD12-2A1 | 3- | 4 | 16 | 1 | K3- | 9 | 1 | IgA |
| | | 74*01 | | | | 20*01 | | | |
| KD12 | KD12- | 3- | 4 | 8 | 0 | K1- | 9 | 0 | IgA |
| | 1F10 | 21*01 | | | | 39*01 | | | |
| KD12 | KD12- | 3- | 4 | 12 | 7 | K3- | 9 | 7 | IgG |
| | 2A10 | 15*07 | | | | 20*01 | | | |
| KD12 | KD12-1G7 | 3- | 4 | 11 | 10 | L1-44*01 | 11 | 6 | IgA |
| | | 66*01 | | | | | | | |

Group 1: KD Mabs previously described to bind Kdpep1¹⁷, shown in this report to have improved binding to Kdpep3. Group 2: KD Mabs generated from 8 different KD patients in 2017-2018 that bind to Kdpep3. Group 3: KD Mabs generated from one KD patient in 2022 that recognize Kdpep3. Members of the IGHV 3-74 gene family are bolded; all others are paralogs of the VH3-74 family.

TABLE 5
Genetic characteristics of KD MAbs prepared in this study

| | | Heavy chain | | | | | Light chain | | | | |
|---|---|---|---|---|---|---|---|---|---|---|---|
| | | | | | CDR3 | Number of | | | CDR3 | Number of | Number of |
| Patient # | | | | | length | mutations | | | length | mutations | plasmablasts |
| (total PB) | MAb | IGHV | IGHD | IGHJ | (aa) | (aa) | IGV | IGJ | (aa) | (aa) | (G, A, M) |
| KD1 | 1H8 | IGHV3-74*01 | IGHD4-17*01 | IGHJ2*01 | 15 | 7 | IGKV3-16*01 | IGKJ2*01 | 9 | 2 | 0, 1, 0 |
| | 2B1 | IGHV3-74*01 | IGHD5-24*01 | IGHJ6*02 | 13 | 4 | IGLV2-11*01 | IGLJ3*02 | 10 | 3 | 0, 1, 0 |
| KD2 | 1D10 | IGHV3-74*01 | IGHD3-9*01 | IGHJ4*02 | 14 | 7 | IGLV2-8*01 | IGLJ2*01 | 11 | 4 | 1, 0, 0 |
| KD3 | 1B6 | IGHV3-74*01 | IGHD4-17*01 | IGHJ4*02 | 14 | 7 | IGLV2-23*02 | IGLJ3*02 | 10 | 9 | 0, 1, 0 |
| | 1C10 | IGHV3-74*01 | — | IGHJ6*02 | 12 | 10 | IGKV3-20*01 | IGKJ2*01 | 10 | 9 | 0, 1, 0 |
| KD5 | 2D1 | IGHV3-74*01 | IGHD7-27*01 | IGHJ6*03 | 9 | 11 | IGLV2-43*01 | IGLJ1*01 | 9 | 3 | 1, 0, 0 |
| | 2D10 | IGHV3-74*01 | IGHD6-19*01 | IGHJ3*02 | 15 | 0 | IGKV3-15*01 | IGKJ1*01 | 10 | 0 | 1, 0, 0 |
| | 2D7 | IGHV3-74*01 | IGHD4-17*01 | IGHJ6*04 | 13 | 6 | IGLV1-44*01 | IGLJ2*01 | 13 | 5 | 1, 0, 0 |
| KD6 | 2H8 | IGHV3-74*01 | IGHD7-27*01 | IGHJ4*02 | 10 | 9 | IGKV2-40*01 | IGKJ3*01 | 9 | 4 | 0, 1, 0 |
| | 1H10 | IGHV3-74*01 | IGHD2-15*01 | IGHJ4*02 | 20 | 11 | IGLV2-23*02 | IGLJ1*01 | 11 | 8 | 0, 1, 0 |
| KD7 | 2H5 | IGHV3-74*01 | IGHD6-19*01 | IGHJ4*02 | 11 | 1 | IGLV1-47*01 | IGLJ3*02 | 11 | 0 | 0, 1, 0 |
| | 1B5 | IGHV3-33*01 | IGHD3-22*01 | IGHJ4*02 | 12 | 6 | IGLV1-51*01 | IGLJ3*02 | 11 | 0 | 0, 1, 0 |
| KD8 | 2E9 | IGHV3-74*01 | IGHD6-25*01 | IGHJ3*02 | 13 | 6 | IGLV1-51*01 | IGLJ3*02 | 11 | 4 | 1, 0, 0 |
| KD9 | 2A4 | IGHV3-64*01 | IGHD6-13*01 | IGHJ4*02 | 13 | 6 | IGLV2-14*01 | IGLJ1*01 | 10 | 2 | 0, 1, 0 |
| | 2H6 | IGHV3-15*01 | IGHD3-22*01 | IGHJ4*02 | 15 | 18 | IGLV3-1*01 | IGLJ1*01 | 9 | 7 | 0, 1, 0 |
| KD10 | 1C3 | IGHV3-30*04 | IGHD5-12*01 | IGHJ4*02 | 14 | 10 | IGLV1-51*01 | IGLJ3*02 | 11 | 4 | 0, 1, 0 |
| | 1G3 | IGHV3-73*01 | IGHD1-14*01 | IGHJ5*02 | 15 | 14 | IGLV5-37*01 | IGLJ2*01 | 11 | 20 | 0, 1, 0 |
| | 1A8 | IGHV3-74*01 | IGHD1-1*01 | IGHJ3*01 | 11 | 8 | IGKV2-29*02 | IGKJ4*01 | 9 | 3 | 1, 0, 0 |
| | 2F6 | IGHV3-74*01 | IGHD3-3*01 | IGHJ4*02 | 10 | 6 | IGLV2-14*01 | IGLJ1*01 | 10 | 10 | 1, 0, 0 |
| KD11 | 2E4 | IGHV3-74*01 | IGHD2-15*01 | IGHJ4*02 | 19 | 11 | IGKV1-6*003 | IGKJ1*01 | 11 | 4 | 0, 1, 0 |
| | 2E5 | IGHV3-74*01 | IGHD5-18*01 | IGHJ4*02 | 13 | 8 | IGKV3-11*01 | IGKJ1*01 | 9 | 3 | 0, 1, 0 |
| KD12 | 1F10 | IGHV3-21*01 | IGHD3-3*01 | IGHJ4*02 | 8 | 0 | IGKV1-39*01 | IGKJ2*02 | 9 | 0 | 0, 2, 1 |
| | 1H2 | IGHV3-21*01 | IGHD2-15*01 | IGHJ4*02 | 8 | 0 | IGKV1-39*01 | IGKJ2*01 | 9 | 0 | |
| | 1A10 | IGHV3-21*01 | IGHD3-10*01 | IGHJ4*02 | 17 | 4 | IGLV1-47*01 | IGLJ2*01 | 11 | 3 | 2, 0, 0 |
| | 2A10 | IGHV3-15*07 | IGHD1-1*01 | IGHJ4*02 | 12 | 7 | IGKV3-20*01 | IGKJ1*01 | 9 | 7 | 1, 0, 1 |
| | 2A1 | IGHV3-74*01 | IGHD6-13*01 | IGHJ4*02 | 16 | 1 | IGKV3-20*01 | IGKJ2*01 | 9 | 1 | 0, 2, 0 |
| | 2H1 | IGHV3-23*01 | IGHD1-14*01 | IGHJ4*02 | 13 | 4 | IGKV3-15*01 | IGKJ4*01 | 9 | 7 | 0, 2, 0 |
| | 2C10 | IGHV3-74*01 | IGHD6-6*01 | IGHJ4*02 | 17 | 3 | IGLV2-23*02 | IGLJ2*01 | 10 | 8 | 0, 2, 1 |
| | 1G7 | IGHV3-66*01 | IGHD2-21*01 | IGHJ4*02 | 11 | 10 | IGLV1-44*01 | IGLJ3*02 | 11 | 6 | 0, 2, 0 |
| | 2F2 | IGHV3-74*01 | IGHD3-10*01 | IGHJ4*02 | 9 | 7 | IGLV1-14*03 | IGLJ1*01 | 10 | 6 | 1, 1, 0 |

Optimizing the epitope recognized by KD MAbs using amino acid substitution scans. To determine if peptides with improved binding to KD MAbs could be identified, the inventors performed substitution matrix analysis using MAbs recognizing the initial KD epitope KPAVIPDREALYQDIDEMEEC (KDpeptide1) (SEQ ID NO: 279). Each position in the peptide was substituted with all other amino acids and binding of KDMAbs was evaluated by peptide array. The inventors performed substitution analysis using a total of 13 KD MAbs, with representative results using 6 KD MAbs shown in FIGS. 2A-2F. The results are depicted in a heat map with red indicating strong binding and blue indicating decreased or no binding compared to the base peptide. The sequential use of the substitution scans led to development of a second KD peptide KPAVIPDREMLIQSIVEMEEC (KDpeptide2) (SEQ ID NO: 280) and then to a third peptide PAVIPDRPMLFQSIVEMEEC (KDpeptide3) (SEQ ID NO: 281). Multiple substitution scans demonstrated the essential or near-essential nature of P⁸, L¹⁰, Q¹², S¹³ and I¹⁴ of KDpeptide3 for binding to the MAbs, with M⁹ and V¹⁵ highly favored and F¹¹ less essential, allowing for substitution by V, T, or I at that position (FIG. 2, amino acid numbering is from KDpeptide3).

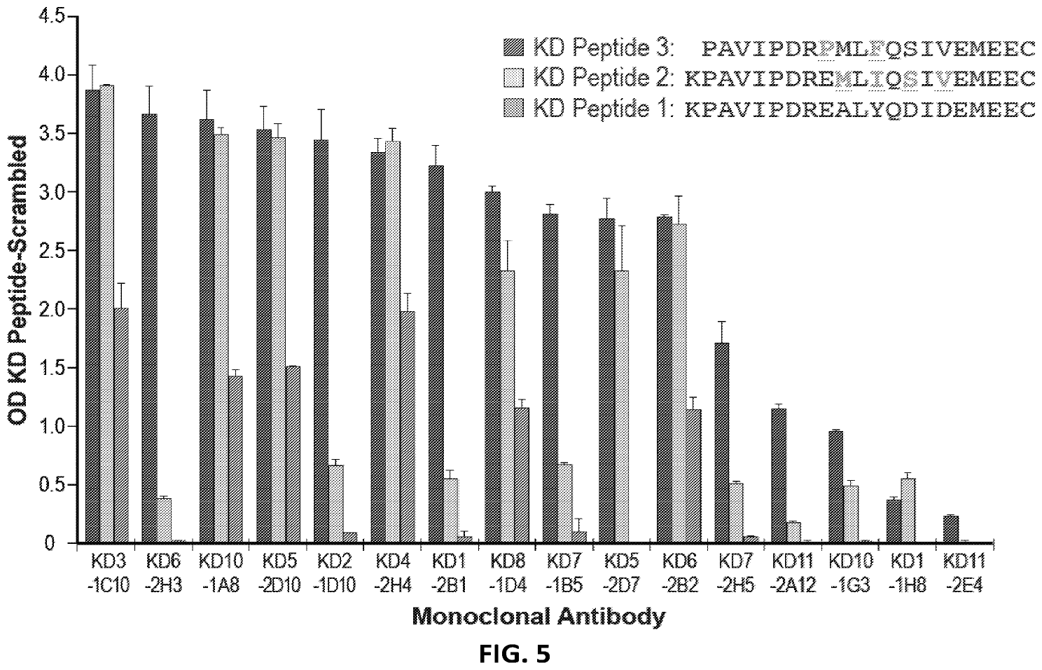

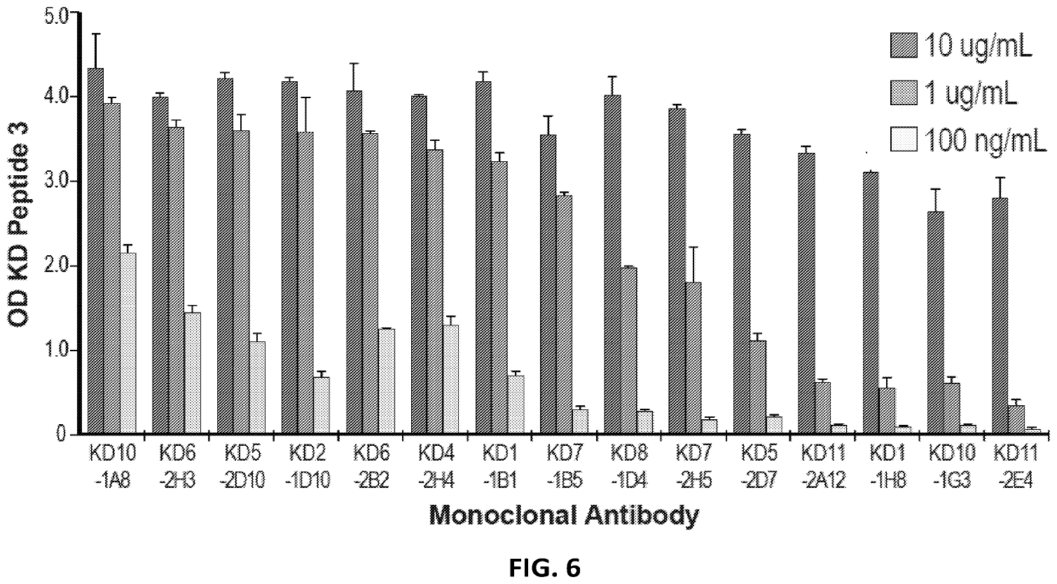

MAbs from children with KD show preferential binding to KDpeptide3. The inventors directly compared binding of KD MAbs to KDpeptide1, KDpeptide2, and KDpeptide3, as shown in FIG. 3A and FIG. 5. Serial dilutions of MAb reactivity against KDpeptide3 are shown in FIG. 6. The three previously identified KD MAbs that bind to KDpeptide1 and 13 newly generated KD MAbs from multiple patients preferentially recognize KDpeptide3 over other peptides. A subset of antibodies demonstrated binding to KDpeptide3 and KDpeptide2, but did not bind to KD peptide 1 (KD1-2B1, KD1-1H8, KD2-1D10, KD5-2D7, KD6-2H3, KD7-2H5, KD7-1B5, KD10-1G3, KD11-2A12, and KD11-2E4) (Table 3, FIGS. 3A, 5, and 6, Table 5). Of note, substitution of P⁸ in KDpeptide3 for E at that position in KDpeptide1 and KDpeptide2 resulted in a significant enhancement of binding for multiple KD MAbs. BLAST analyses of the core sequence PMLF(V,T)QSIV (SEQ ID NO: 282) did not yield human protein hits. Because The inventors have previously demonstrated that KD inclusion bodies identified by KD MAbs contain RNA and not DNA⁸, the inventors also performed BLASTp analyses restricted to RNA viruses, yielding remote hits to diverse RNA viruses. These analyses were largely uninformative because of the limitations of the short core sequence length of about 8 amino acids, similar to the length of other linear epitopes bound by antibodies²², and because the KD agent is likely one whose sequence is not present in established databases. Moreover, acute plasmablast responses to viral infection are often targeted at viral surface proteins, which are less conserved between viral families and can even be divergent between different viral genotypes of the same virus, making it less likely that homology to a known virus could be readily identified^{23-25.}

The inventors also prepared mouse-Fc fusion proteins containing three copies of KDpeptide1 and KDpeptide3 for use in Western blot assays with KD MAbs to assess binding of the MAbs to these fusion proteins. Western blot results using the MAbs were comparable to ELISA using the peptides themselves. KD MAbs demonstrated enhanced binding to fusion proteins containing KDpeptide3 compared with those containing KDpeptide1 (KD4-2H4, FIG. 3B). Some MAbs recognized fusion proteins containing KDpeptide3 but did not recognize those containing KDpeptide1, similar to the ELISA results (KD6-2H3, FIG. 3B and FIG. 5).

Importantly, all KD MAbs that were initially discovered to bind to KDpeptide1 showed stronger binding to KDpeptide3 (FIG. 3a, p value <0.01 for all MAbs), indicating that KDpeptide3 is a common epitope in the KD response. These results suggest that the KDpeptide3 sequence more closely resembles the sequence of the antigen in the KD triggering agent (either as a linear sequence or as a mimotope) than does the KDpeptide1 sequence.

KD MAbs binding to KDpeptide3 from 10 of 11 KD patients use heavy chain VH3-74 or its paralogs. All MAbs binding to KDpeptide3 derived from VH3-74 or its paralog families with similarity scores of ≥82%²¹ (Table 4, Table 5). These MAbs were identified in 10 of 11 children with KD, including all of 8 children who developed coronary aneurysms (Tables 3 and 4). The single child (KD9) from whom the inventors did not identify a MAb that binds to any of the three peptides did not have plasmablasts encoding VH3-74 or paralogs VH3-33 or VH3-72 among the 81 single cells sequenced. This patient fulfilled clinical diagnostic criteria for KD but did not develop coronary artery abnormalities.

The genetic features of the MAbs prepared for this report are detailed in Table 5. MAbs recognizing KDpeptide3 differed in CDR3 sequence, with lengths varying from 11-19 amino acids, and with 0-15 amino acid mutations from germline. D genes D4-17*01, D3-9*01, D6-13*01, and D6-19*01, and light chains L2-11*01, L1-44*01, K3-15*01, K3-20*01, and K1-5*03 were used by two or more MAbs in the dataset (Table 5). Overall, 10 of 15 (67%) VH3-74 MAbs and 2 of 3 (67%) VH3-33 MAbs that the inventors produced from plasmablasts from 11 children with KD recognized KDpeptide3 (Table 4 and Table 5). The inventors note that neither 3 VHI nor 4 VH4 antibodies from clonally expanded plasmablasts from the original 11 KD patients recognized KDpeptide1, 2, or 3, nor did 5 VH3-74 MAbs from these patients, suggesting that the response to KD likely includes additional epitopes that the inventors have not yet identified and/or that some VH3-74 MAbs circulating in the plasmablast pool were not responding to KD (Table 5).

The prevalence of VH3-74 MAbs from children with KD that bind to KDpeptide3 define a convergent plasmablast response to a specific protein epitope in KD.

Plasmablast analysis of KD patient 12 also yields MAbs binding to KDpeptide3. Our initial study of KD plasmablasts included children with KD diagnosed in 2017-2018¹⁷. To determine if the antigen that includes the KDpeptide3 sequence was also recognized by a KD patient presenting 5 years following our initial study, the inventors sequenced 159 single plasmablasts from KD12, an infant with classic KD who developed a giant coronary artery aneurysm in 2022. In this patient, SARS-CoV-2 antibody was negative and there was no lymphopenia, hypotension, or myocardial dysfunction to suggest MIS-C. Nine sets of clonally expanded plasmablasts were identified in this child's peripheral blood (Table 5). These included 3 sets of clonally expanded VH3-74 plasmablasts, two of which included plasmablasts of more than one isotype within the set, compatible with isotype switching during an acute response to infection. One of these VH3-74 plasmablasts, KD12-2A1, recognized KDpeptide3 but not KDpeptide1 or KDpeptide2 (Table 4, FIG. 3A). MAbs made from three additional clonally related sets of plasmablasts, KD12-1F10, KD12-1G7, and KD12-2A10, also recognized KDpeptide3 albeit with lower binding; these MAbs were of the VH3-21, VH3-66, and VH3-15 families, respectively, which are paralogs of VH3-74 with similarity scores of ≥82% 21 (Table 4, Table 5).

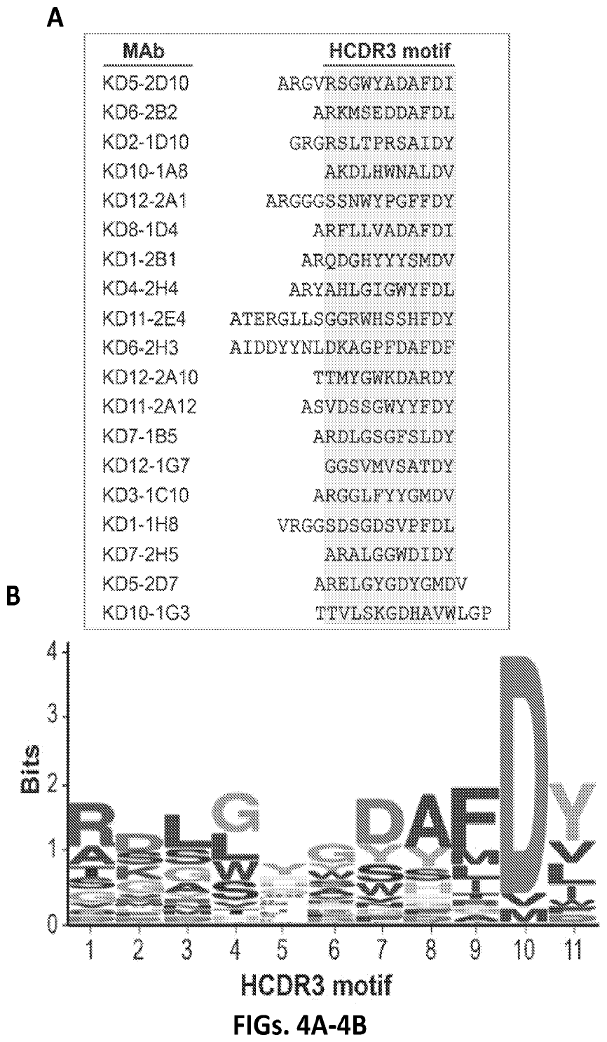

KD MAbs binding to KD peptide3 share a common CDR3 epitope. The inventors found that MAbs from KD patients 1-12 that recognize KDpeptide3 did not have the same CDR3 sequences (FIG. 4A). However, motif-based sequence analysis of the CDR3 amino acid sequences (https://meme-suite.org/meme/) revealed a statistically significant common motif at 2.3e-6 (FIG. 4B). Of the 20 KD MAbs recognizing KDpeptide3 (Table 4), 19 share the motif, with KD12-1F10 the only exception. These results provide further support for a convergent VH3-74 antibody response to the KDpeptide3 protein antigen in children with KD presenting over a 5-year time period.

KD MAbs do not demonstrate cross-reactive binding to SARS-CoV-2 proteins by ELISA. Because of some clinical similarities between SARS-CoV-2-induced MIS-C and KD, some investigators have suggested that KD might be caused by a virus with homology to SARS-CoV-2. To determine if KD MAbs were cross-reactive with SARS-CoV-2 spike or nucleocapsid proteins, the inventors performed ELISA of KD MAbs against these proteins. Control antibodies to these proteins gave positive results, while none of 13 KD MAbs reacted with these proteins, including¹¹ that react with KDpeptide3, and 2 others whose targets remain unknown (data not shown).

### DISCUSSION

KD is characterized by significant inflammation of a variety of organs and tissues, most notably the coronary arteries, but the inciting agent of this response has remained a mystery. The differential diagnosis of KD is wide, since the clinical features are shared by many infectious and inflammatory conditions of childhood, hampering diagnosis and institution of appropriate treatment 1. Because no specific infectious etiologic agent has been identified as the cause of KD to date, diverse triggering etiologies have been suggested. However, this theory does not explain the restricted age group affected, the rarity of recurrence, and the worldwide reports of outbreaks and epidemics of illness, which are much more compatible with a single causative agent or group of closely related agents that results in lifelong immunity following infection in most cases. In the present study, the inventors report a refined protein epitope targeted by a convergent VH3-74 antibody response in 11/12 children with KD. These findings are consistent with immune response to an antigen derived from the same causative agent in these 11 patients, as summarized in FIG. 1. The one patient in whom this response was not identified had typical features of the illness but did not develop coronary artery abnormalities. The possibilities for a lack of detection of a similar response in this single patient include an insufficient number of plasmablasts sequenced, misdiagnosis of another illness of childhood as KD, or a different KD triggering pathogen. Convergent antibody responses as observed in this study are characteristic of responses to specific antigens of infectious agents^{19,20,26} These data support our hypothesis of a single, presently unidentified, respiratory virus as the predominant or sole cause of KD and provide a direction for the development of serologic assays^{11,17,27.}

Because of some clinical similarities between KD and MIS-C and the observation that some children with MIS-C can have dilation of the coronary arteries during their acute illness, some investigators have postulated that a virus closely related to SARS-CoV-2 might be the cause of KD. However, coronary artery dilation arising from MIS-C is mild, short-lived, and peaks during the acute febrile illness, features that are distinct from KD and similar to what has been observed in other inflammatory conditions associated with marked cytokine release such as systemic onset juvenile idiopathic arthritis²⁸. Moreover, autopsy studies on fatal MIS-C cases to date have not revealed coronary artery inflammation, which is the hallmark of KD^{1,29-33}. The inventors tested KD MAbs to determine if they showed cross-reactivity with SARS-CoV-2 proteins, with negative results.

Determining the nucleic acid sequence of the putative KD viral agent is a research priority. Identification of a novel virus in this patient population is particularly challenging. The target tissues of the disease, the coronary arteries, are unavailable to the researcher in the living patient. The disease affects very young children, limiting clinical samples available for research. It is unlikely that the KD agent is present in blood samples at the time of clinical presentation, because high throughput sequencing of blood samples has not yielded the agent. Fatalities generally occur weeks into the illness as a complication of coronary artery inflammation, a time when the immune system may have cleared the pathogen or reduced it to a very low level in KD tissues. If the agent is dissimilar to known viruses, then its identification among unassigned sequences in a high-throughput sequencing dataset could be very challenging. Moreover, available KD tissues containing virus-like inclusion bodies are virtually all formalin-fixed and paraffin-embedded, yielding fragmented RNA that could be resistant to assembly of a genome.

These results identify a specific protein epitope targeted by VH3-74 plasmablasts encoding a common CRD3 motif in children with KD. The findings support a research focus toward identification of a predominant etiologic agent for KD and provide insights into the pathogenesis of this potentially fatal illness of childhood.

This work reports a convergent VH3-74 plasmablast response to a specific protein epitope in 11 children with KD. The inventors identified this epitope using substitution matrix analyses of the epitope in our original report¹⁷. Because convergent antibody responses are typical of B cell response to distinct antigens of specific pathogens, the refined epitope likely represents either a linear epitope or a mimotope of an antigen derived from the triggering agent of KD^{19,20} These results strongly favor one predominant cause of KD, providing significant progress toward identifying the etiology and pathogenesis.

### Methods

Patients. This study was approved by the Institutional Review Board of the Ann and Robert H. Lurie Children's Hospital of Chicago, and patients were enrolled following informed consent. Peripheral blood was obtained from KD patients on day 8-24 after fever onset. Patients KD1 through KD11 presented in April 2017 through July 2018, and were previously reported¹⁷. Patient KD 12 presented in February 2022. Clinical and plasmablast data on the 12 patients are described in Table 3.

Flow Cytometry. CD3⁻CD19⁺CD38⁺⁺CD27⁺⁺ peripheral blood mononuclear cells were gated and single cells sorted into individual wells of 96-well plates, as previously described 17.

Amplification, Sequencing, and Cloning of Immunoglobulin Variable Regions. Reverse transcription and polymerase chain reaction amplification of heavy and light chain variable genes were performed as previously described¹⁷. Light chains were cloned into human immunoglobulin K and A light chain expression vectors³⁴ and heavy chains were cloned into human γ1 and rabbit γ (pFUSEss vectors, Invivogen) heavy chain expression vectors, to enable production of human and rabbit versions of the MAbs. Heavy and light chains of the MAbs produced for this study have been submitted to GenBank with accession numbers OP207904 through OP207952.

Antibody Production and Analysis. Antibodies were produced by transfection of 293F suspension cells using a 1.5:1 ratio of light chain:heavy chain DNAs and Freestyle MAX reagent, and purified over protein A agarose beads (ThermoFisher Scientific).

Substitution Analysis. Substitution analysis was performed on peptides recognized by KD MAbs by creating a peptide array that includes stepwise substitution of all amino acid positions of the peptide with all 20 amino acids, to determine the amino acids that yielded optimal antibody binding (PEPperPRINT, www.pepperprint.com).

ELISA for Binding of peptides to KD Monoclonal Antibodies. Immulon 2 HB 96-well plates (ThermoFisher) were coated with 800 ng of synthetic peptides (Anaspec) per well and incubated with rabbit KD MAbs at 10, 1, and 0.1 μg/mL in triplicate followed by horseradish peroxidase (HRP)-labeled goat anti-rabbit antibody at 1:2500 (Southern Biotech). Absorbance at 450 nm was determined on a Multiskan FC spectrophotometer after addition of ultra 3,3′,5,5′-tetramethylbenzidine followed by 1.5 M sulfuric acid solution. Absorbance of the KD peptides were recorded after subtraction of results obtained using scrambled versions of the peptides. An OD reading more than ten times the background reactivity of scrambled peptides was considered positive; OD readings of KD MAbs with scrambled peptides and negative MAbs with KD peptides were consistently ≤0.05. The inventors used two-sample t-tests to compare the differences of intensity between KD Peptide 1 and KD Peptide 3 for each monoclonal antibody.

ELISA for reactivity of KD MAbs with SARS-CoV-2 proteins. Immulon 2 HB plates were coated with SARS-CoV-2 spike hexapro at 50 ng/well (plasmid graciously provided by F. Krammer) or full-length nucleocapsid protein 100-μL/well (Invivogen, his-sars2-n) in coating buffer (50 mM sodium carbonate/bicarbonate, pH 9.6) and incubated overnight at 4° C. KD rabbit MAbs were applied at 2 μg/mL in triplicate followed by HRP-labeled goat anti-rabbit IgG antibody (Southern Biotech) at 1:4000. Positive controls were rabbit antibody to SARS-CoV-2 spike protein (Thermo Scientific 703971) and rabbit antibody to SARS-CoV-2 nucleocapsid (Thermo Scientific MA5-36086). Absorbance was determined as above.

Western Blot Assay using Mouse Fc-Concatemerized KD peptide Fusion Proteins. The inventors optimized the nucleotide sequence that codes for KD peptide sequences for expression in 293F cells and prepared multimers of KD peptide sequences linked by short spacers. For KDpeptide1, the sequence was: AGKPAVIPDREALYQDIDEMEECLDEAGKPAVIPDREALYQDIDEMEECLDEAGKPAVI PDREALYQDIDEMEECLD (SEQ ID NO: 279). For KDpeptide3, the sequence was: AGVIPDRPMLFQSIVEMEECLDEAGVIPDRPMLFQSIVEMEECLDEAGVIPDRPMLFQSIV EMEECLD (SEQ ID NO: 281). The sequences were cloned into pINFUSE-mlgG2b-Fc2 (Invivogen), and the fusion protein prepared by transfection in 293 cells followed by protein A agarose bead purification. Western blot assays were performed using 100 ng of each construct and electrophoresis on 12% Tris-glycine gels (Biorad) with transfer to PVDF membrane (Fisher). After blocking the membranes, KD MAbs at 0.1 μg/mL were incubated with membranes overnight at 4° C. Following incubation, membranes were washed and incubated with HRP-labelled goat anti-human IgG (ThermoFisher A18811) at a dilution of 1:5000 and developed using Supersignal West Femto Substrate (ThermoFisher).

TABLE 6
Informal Sequence Listing

| SEQ ID | |
|---|---|
| NO | Sequence |
| 1 | EVQLVESGGGLVQPGGSLRLSCAASGFTFSSYWMHWVRQAPGKGLV |
| | WVSRINSDGSSTSYADSVKGRFAISRDNAKNTLYLQMNSLRAEDTAV |
| | YYCARALGGWDIDYWGQGTLVTVS |
| 42 | GFTFSSYW |
| 75 | INSDGSST |
| 4 | ARALGGWDIDY |
| 5 | QSVLTQPPSASGTPGQRVTISCSGSSSNIGSNYVYWYQQLPGTAPKLLI |
| | YRNNQRPSGVPDRFSGSKSGTSASLAISGLRSEDEADYYCAAWDDSLS |
| | GGVFGGGTKLTVLG |
| 6 | SSNIGSNY |
| 7 | RNN |
| 8 | AAWDDSLSGGV |
| 9 | QSVLTQPPSVSAAPGQKVTISCSGGSSSIGNNYVSWYQLLPGTAPKLLI |
| | YDNNKRPSGIPDRFSGSKSGTSATLGITGLQTGDEADYYCGTWDSGLS |
| | AGVFGGGTKLTVLG |
| 10 | SSSIGNNY |
| 11 | DNN |
| 12 | GTWDSGLSAGV |
| 13 | VQLVESGGGVVQPGRSLRLSCAASGFTFSDYAVHWVRQAPGKGLEW |
| | AALISNDGDFKYYADAVKGRFTISRDNSNNTLYLQMNSLRTEDTAVY |
| | YCARERGRDKVFPPGYWGQGTLVTVS |
| 14 | GFTFSDYA |
| 15 | SNDGDFK |
| 16 | ARERGRDKVFPPGY |
| 17 | QSVLTQPPSVSAAPGQKVTISCSGSSSNIGNNYVSWYQQLPGTAPKLLI |
| | YDNNKRPSGIPDRFSGSKSGTSATLGITGLQTGDEADYYCGTWDSSLS |
| | AEVFGGGTKLTVLG |
| 18 | SSNIGNNY |
| 19 | DNN |
| 20 | GTWDSSLSAEV |
| 21 | VQLVESGGGVVQPGRSLRLSCEASGFTFRSRAMHWVRQAPGKGLGW |
| | VAVIWYDGSNKYYADSVKGRFTISRDNSRNTLYLQMNSLRAEDTAVY |
| | YCARDLGSGFSLDYWGQGTLVTVS |
| 22 | GFTFRSRA |
| 23 | IWYDGSNK |
| 24 | ARDLGSGFSLDY |
| 25 | QAVVTQEPSLTVSPGGTVTLTCASSTGAVTSAHSPHWFRQKPGQAPRT |
| | LIYDTSNKPSWTPARFSGSLLGGKAALTLSGAQPEDEAEYYCLLSNSG |
| | VHFLFGGGTRLTVLG |
| 26 | TGAVTSAHS |
| 27 | DTS |
| 28 | LLSNSGVHFL |
| 29 | EVQLVQSGAEVKKTGSSVKLSCTATGYTFTYRYLHWVRQAPGQALE |
| | WMGYITIYNGDTNYAQKFQDRVTISRDMSLSTVYMELSSLTSEDTAM |
| | YFCVRSALYGENAFDFWGQGTMVTVS |
| 30 | GYTFTYRY |
| 31 | ITIYNGDT |
| 32 | VRSALYGENAFDF |
| 33 | EVQLVESGGGSVQPGGSLRLSCAASGFTFSNYWMHWVRQAPGKGLV |
| | WVSRLNIDGSNTFYADSVKGRFTISRDNAKNTLYLQMNSLRAEDTAV |
| | YYCVRGGSDSGDSVPFDLWGRGTLVTVS |
| 78 | GFTFSNYW |
| 35 | LNIDGSNT |
| 36 | VRGGSDSGDSVPFDL |
| 37 | EIVMTQSPATLSVSPGERATLSCRASQSVSSNLAWYQQNPGQAPRLLIY |
| | GASTRATGIPARFSGSGSGTEFTLTISSLQSEDFAVYYCQHYNNWPFTF |
| | GQGTKVEIK |
| 38 | QSVSSN |
| 39 | GAS |
| 40 | QHYNNWPFT |
| 41 | EVQLVESGGGLVQPGGSLRLSCAASGFTFSSYWMHWVRQAPGKGLV |
| | WVSRINNDGSSTGYADSVEGRFTISRDNAKNTLYLQINSLRAEDTAVY |
| | YCARQDGHYYYSMDVWGQGTTVTV |
| 42 | GFTFSSYW |
| 43 | INNDGSST |
| 44 | ARQDGHYYYSMDV |
| 45 | QSALTQPRSVSGSPGQSVTISCTGTSSDVGGYNYVSWYQQHPGKAPKL |
| | MIYDVSERPSGVPARFSGSKSGNTASLTISGLQAEDEADYYCCSYAGN |
| | FRWVFGGGTKLTVL |
| 46 | SSDVGGYNY |
| 47 | DVS |
| 48 | CSYAGNFRWV |
| 49 | EVQLVESGGGLVQPGGSLKLSCAASGFSFSGAAMHWVRQSSGRGLEW |
| | LGRIRSKTNDYATAYAESLHGRFTISRDDAKNTAYLQMNRLKSEDTAI |
| | YYCTTVLSKGDHAVWLGPWGPGTLVTV |
| 50 | GFSFSGAA |
| 51 | IRSKTNDYAT |
| 52 | TTVLSKGDHAVWLGP |
| 53 | QPVLTQPPSSSASPGESARLTCTLPSDISVAASDIYWYQQKAGSPPNFLL |
| | YDPSDSHKGQDSGVPSRFSGSRDGSANSGFLLISGVQSEDEADYYCMV |
| | WPPNTVGVVFGGGTTLTV |
| 54 | SDISVAASD |
| 55 | DPSDSHK |
| 56 | MVWPPNTVGVV |
| 57 | QAVVTQEPSLTVSPGGTVTLTCASSTGAVTSGYYPNWFQQKPGQAPR |
| | ALIYSTSNKHSWTPARFSGSLLGGKAALTLSGVQPEDEAEYYCLLFYG |
| | GVHVFGAGTKVTVLG |
| 58 | TGAVTSGYY |
| 59 | STS |
| 60 | LLFYGGVHV |
| 61 | EVQLVESGGDLVQPGGSLRLSCAASGFTFSTYWMHWVRQAPGQGLV |
| | WVSRINGNGRITNYADSVKGRFTVSRDNAKNTVDLQMNSLRAEDTAV |
| | YYCARINEWGDVWGKGTTVTVS |
| 62 | GFTFSTYW |
| 63 | INGNGRIT |
| 64 | ARINEWGDV |
| 65 | DIVMTQTPLSLSVTPGQPASISCKSSQSLLHNDGKTFLHWYLQKPGQSP |
| | QLLIYEVSSRFSGVPDRFSGSGSGTDFTLKISRVEAEDVGVYYCMQGIH |
| | LPPTFGGGTKVEIKR |
| 66 | QSLLHNDGKTF |
| 67 | EVS |
| 68 | MQGIHLPPT |
| 69 | EVQLVESGGGLVQPGGSLRLSCAASGFTFSSYWMEWVRQAPGKGLV |
| | WVSHISSDGSVTRYVDSVKGRFTISRDNAKNTLYLQMNSLRAEDTGV |
| | YYCAKDLHWNALDVWGQGTMVTVS |
| 42 | GFTFSSYW |
| 70 | ISSDGSVT |
| 71 | AKDLHWNALDV |
| 72 | EIVMTQSPATLSVSPGERATLSCRASQSVSSNLAWYQQKPGQAPRLLIY |
| | GASTRATGIPARFSGSGSGTEFTLTISSLQSEDFAVYYCQQYNNWPPWT |
| | FGQGTKVEIKR |
| 38 | QSVSSN |
| 39 | GAS |
| 73 | QQYNNWPPWT |
| 74 | EVQLVESGGGLVQPGGSLRLSCAASGFTFSSYWMHWVRQAPGKGLV |
| | WVSRINSDGSSTSYADSVKGRFTISRDNAKNTLYLQMNSLRAEDTAVY |
| | YCARGVRSGWYADAFDIWGQGTMVTVS |
| 42 | GFTFSSYW |
| 75 | INSDGSST |
| 76 | ARGVRSGWYADAFDI |
| 77 | EVQLVESGGGLVQPGGSLGLSCAASGFTFSNYWMHWVRQAPGKGLV |
| | WVSRINSDGSDTSYADSVKGRFTISRDNAENTLYLHMSSLRAEDTAVY |
| | YCARELGYGDYGMDVWGKGTTVTVS |
| 78 | GFTFSNYW |
| 79 | INSDGSDT |
| 80 | ARELGYGDYGMDV |
| 81 | QSVLTQPPSASGTPGQRVTISCSGSSSNIGSHSVIWYQQLPGTAPKLLVY |
| | SDNQRPSGVPDRFSGSKSGTSASLAISGLQSEDEADYYCAAWDDSLNH |
| | LHVVFGGGTKLTVL |
| 82 | SSNIGSHS |
| 83 | SDN |
| 84 | AAWDDSLNHLHVV |
| 85 | EVQLVESGGGLVQPGGSLRLSCAASGFIFSNYWMHWVRQAPGEGLV |
| | WVSRINKDGSSTFYADSVKGRFTISRDNAKNTLYLQMNSLRAEDTAV |
| | YYCTRDFDFWSGYWGQGTLVTVS |
| 86 | GFIFSNYW |
| 87 | INKDGSST |
| 88 | TRDFDFWSGY |
| 89 | QSALTQPASVSGSPGQSITISCTGTSSDVGGCEYVSWYQQHPGKVPKLII |
| | YEVSNRPSGVSNRFSGSKSGNTASLTVSGLQAEDEADYFCSSCTTSGSY |
| | VFGAGTKVTVV |
| 90 | SSDVGGCEY |
| 67 | EVS |
| 91 | SSCTTSGSYV |
| 92 | EVQLVESGGGVVQPGESLRLSCAASGVTFSSYWMHWVRQVPGKGLV |
| | WVARINIDGTSTTYADCVKGRFTISRDNAKNTLYLQMSSLRAEDTAVY |
| | YCATERGLLSGGRWHSSHFDYWGQGTLVTVS |
| 93 | GVTFSSYW |
| 94 | INIDGTST |
| 95 | ATERGLLSGGRWHSSHFDY |
| 96 | DIQMTQSPSTLSASVGDRVTITCRASQSISSWLAWYQQKPGKAPKVLIY |
| | KTSSLESGVPSRFSGSGSGTEFTLTISSLQPDDFATYYCQQYDSSSLTWT |
| | FGQGTKVEIK |
| 97 | QSISSW |
| 98 | KTS |
| 99 | QQYDSSSLTWT |
| 100 | EVQLVESGGGLVQPGGSLRLSCAASGFTFSRFWMHWVRQVPGKGLV |
| | WISRINSDATSSSYADSVKGRFTISRDNAKNTLFLQMNSLRAEDTAVY |
| | YCATSNTALVYLPENWGQGTLVTVS |
| 101 | GFTFSRFW |
| 102 | INSDATSS |
| 103 | ATSNTALVYLPEN |
| 104 | EIVLTQSPATLSLSPGERATLSCRASQSVSDYLAWYQQKPGQAPRLLIY |
| | DASNRATGIPARFSGGGSGTDFTLTISSLEPEDFAVYYCQQRSYWPPTF |
| | GQGTKVEIK |
| 105 | QSVSDY |
| 106 | DAS |
| 107 | QQRSYWPPT |
| 108 | QSALTQPPSASGSPGQSVTITCTGTSSDVGGYNYVSWYQQHPGKAPKL |
| | MIYEVSKRPSGVPDRFSGSKSGNTASLTVSGLQAEDEADYHCSSFAGD |
| | NNSPVFGGGTKLTVL |
| 46 | SSDVGGYNY |
| 67 | EVS |
| 109 | SSFAGDNNSPV |
| 110 | EVQLVESGGGLIHPGGSLRLSCAASGFTFSSYWMHWVRQAPGKGLVW |
| | VSHIKSDGSNTIYADSVKGRFTISRDNAKNTLYLQMNSLRAEDTAVYY |
| | CGRGRSLTPRSAIDYWGQGTLVTV |
| 42 | GFTFSSYW |
| 111 | IKSDGSNT |
| 112 | GRGRSLTPRSAIDY |
| 113 | LVESGGGLVQPGGSLRLSCAASGFTFSSYAMHWVRQAPGKGLEFASAI |
| | SSDGGTTYYANSVKGRFTISRDNSKNTLYLQMGSLRDEDMAVYYCAR |
| | DDLSTSWDLDYWGQGTLVTV |
| 2 | GFTFSSY |
| 114 | ISSDGGTT |
| 115 | ARDDLSTSWDLDY |
| 116 | QSALTQPASVSGSPGQSITISCTGTSSDVGGYNYVSWYQQHPGKAPKL |
| | MIYDVSNRPSGVSNRFSGSKSGNTASLTISGLQAEDEADYYCSSYTISST |
| | NVFGTGTKVTVL |
| 46 | SSDVGGYNY |
| 47 | DVS |
| 117 | SSYTISSTNV |
| 118 | QSALTQPASMSGSPGQSITISCTGTSSDVGAHNFISWYQQHPGKAPKLM |
| | IFEVNKRPSGVSNRFSGSKSGNTASLTISGLQAEDEADYFCCSYAGYST |
| | WVFGGGTKLTVL |
| 119 | SSDVGAHNF |
| 120 | EVN |
| 121 | SYAGYSTWV |
| 122 | EVQLVESGGGVVQPGGSLRLSCEVSGFIFSSYWMHWVRQVAGKGLV |
| | WVSRINSDGSSTSYADSVKGRFTISRDNAKNTLNLQMNFLRAEDTAVY |
| | YCARGGDHGDYGFFESWGQGTLVTV |
| 123 | GFIFSSYW |
| 75 | INSDGSST |
| 124 | ARGGDHGDYGFFES |
| 125 | EVQLVESGGGLVQPGGSLRLSCAASGFTFSNHWMHWVRQAPGKGLL |
| | WVSRIDTGGSTTNYADSVKGRFTISRDSAKNTVYLQMNSLRAEDTAV |
| | YYCARGGLFYYGMDVWGQGTTVTV |
| 126 | GFTFSNHW |
| 127 | IDTGGSTT |
| 128 | ARGGLFYYGMDV |
| 313 | EIVLTQSPGTLSLSPGERATLSCRASQTSSSTSLAWYQQKRGQAPRLLIY |
| | GASRRATGIP |
| | DRFSGSGSGTDFTLTISRLEPEDFAVYYCQQFSGSPAYTFGQGTKVEIK |
| 129 | QTSSSTS |
| 39 | GAS |
| 130 | QQFSGSPAYT |
| 131 | DIVMTQTPLSLPVTPGEPASISCRSSQSLLDSADGNTYLDWYLQKPGQS |
| | PQLLIHTLSHRASGVPDRFSGSGSGTDFTLKISRVEAEDVGVYYCMQRI |
| | AFPVTFGPGTKVDIK |
| 132 | QSLLDSADGNTY |
| 133 | TLS |
| 134 | MQRIAFPVT |
| 135 | EVQLVESGGGLVQPGGSLRLSCAASGFTFSNYWMHWVRQAPGEGLE |
| | WVSRINSDGNKVSYADSVRGRFTISRDNAKNTLYLQMNSLTGEDTAV |
| | YYCARSNWGSADYWGQGTLVTV |
| 78 | GFTFSNYW |
| 136 | INSDGNKV |
| 137 | ARSNWGSADY |
| 138 | EVQLVESGGGSVQPGGSLRLSCAASGFTFSRYWMHWVRQAPGEGLV |
| | WVSRINSDETDKLYADSVKGRFSIFRDNAKNTLYLQMNRLRAEDTAV |
| | YYCARDREDVVVGPATQHTIFNSWGQGTLVTV |
| 139 | GFTFSRYW |
| 140 | INSDETDK |
| 141 | ARDREDVVVG |
| 142 | QSALTQPASVSGSPGQSITISCTGTSSDVGSSNFVSWYQQHPGKAPKLIL |
| | YEVSKRPAGVSSRFSGSRSGNTASLTISGAQAEDEADYSCCSTSSVGTL |
| | YVFGTGTKVTVL |
| 143 | DVGSSNF |
| 67 | EVS |
| 144 | CSTSSVGTLYV |
| 145 | QSVLTQPPSVSAAPGQKVTISCSGSNYNIGNNYVSWYQRLPGTAPKLLI |
| | YDNNKRPSGIPDRFSGSKSGTSATLGITGLQTGDEADYYCGTWDSSLR |
| | AGVFGGGTKLTVL |
| 146 | NYNIGNNY |
| 11 | DNN |
| 147 | GTWDSSLRAGV |
| 148 | EVQLVESGGGLVQPGGSLRLSCAASGFTFSRHWMHWVRQAPGKGLV |
| | WVSRINSDGSSTSNADSVKGRITISRDNGKNTLYLQMNSLRAEDTAVY |
| | YCAREIASGTDAFDIWGQGTMVTV |
| 149 | GFTFSRHW |
| 75 | INSDGSST |
| 150 | AREIASGTDAFDI |
| 151 | EVQLVESGGGLVKPGESLRLSCVGSGFTLTNAWMIWVRQTSGKGLEW |
| | VGRIKSKIDGGAIDYGAPVKGRFTISRDDTKNTVYLQMNSLQTDDTGV |
| | YFCTTDRYSTGYYGMDDYWGQGTLVTV |
| 152 | GFTLTNAW |
| 153 | IKSKIDGGA |
| 154 | TTDRYSTGYYGMDDY |
| 155 | SYELTQPPSVSVSPGQTATITCSGDKLGDKYSFWYQQKPGQSPVVVIY |
| | QDSKRPSGIPERFSGSNSGNTATLTISGTQAMDEADYYCQVWDTDSAV |
| | FGTGTRVTV |
| 156 | KLGDKY |
| 157 | QDS |
| 158 | QVWDTDSAV |
| 159 | DIQMTQSPSSLSASVGDRVTITCRASQSISSYLNWYQQKPGKAPKLLIY |
| | AASSLQSGVPSRFSGSGSGTDFTLTISSLQPEDFATYYCQQSYSTPYTFG |
| | QGTKVEIK |
| 160 | QSISSY |
| 161 | AAS |
| 162 | QQSYSTPYT |
| 163 | EVQLVESGGGLVKPGGSLRLSCAASGFTFSSYSMNWVRQAPGKGLEW |
| | VSSISSSSSYIYYADSVKGRFTISRDNAKNSLYLQMNSLRAEDTAVYYC |
| | ARDTISRYWGQGTLVTV |
| 164 | GFTFSSYS |
| 165 | ISSSSSYI |
| 166 | ARDTISRY |
| 167 | EVQLVESGGGLVKPGGSLRLSCAASGFTFSSYSMNWVRQAPGKGLEW |
| | VSSISGSSSYMHYAESVKGRFTISRDNAKNSLYLQMNSLRAEDTAVYY |
| | CASTGIITYYYASGVPDYWGQGTLVTV |
| 164 | GFTFSSYS |
| 168 | ISGSSSYM |
| 169 | STGIITYYYASGVPDY |
| 170 | QSVLTQPPSASGTPGQRVTMSCSGSSSNIGRNYVYWYQQLPGTAPKLL |
| | IYRNNQRPSGVPDRFSGSKSGTSVSLAISGLRSEDEADYYCAAWDDSLS |
| | GVVFGGGTKLTVL |
| 171 | SSNIGRNY |
| 7 | RNN |
| 172 | AAWDDSLSGVV |
| 173 | EIVLTQSPGTLSLSPGERATLSCRASQSVSSNLAWYQQKPGQAPRVLIY |
| | GASSRATGIPDRESGSRSGTDFTLTISRLEPEDFAVYYCQRYDNSPRTFG |
| | QGTKVEIK |
| 38 | QSVSSN |
| 39 | GAS |
| 174 | QRYDNSPRT |
| 175 | EVQLVESGGGLVKPGGSLRLSCAASGFTFINAWMNWVRQAPGKGLE |
| | WVGLIKSKTDGGTIDYAAPVKGRFTISRDDSEKMLYLQMDSLKTEDTA |
| | VYYCTTMYGWKDARDYWGQGTLVTV |
| 176 | GFTFINAW |
| 177 | IKSKTDGGT |
| 178 | TTMYGWKDARDY |
| 179 | EIVLTQSPGTLSLSPGERATLSCRASQSVSSSYLAWYQQKPGQAPRFLI |
| | YGASSRATGIPDRFSGSGSGTDFTLTISRLEPEDFAVYYCQQYGSSPYTF |
| | GQGTKVEIK |
| 180 | QSVSSSY |
| 39 | GAS |
| 181 | QQYGSSPYT |
| 182 | EVQLVESGGGLVQPGGSLRLSCAASGFTFSSYWMHWVRQAPGKGLV |
| | WVSRIKSDGSSTSYADSVKGRFTISRDNAKNTLYLQMNSLRAEDTAVY |
| | YCARGGGSSNWYPGFFDYWGQGTLVTV |
| 42 | GFTFSSYW |
| 183 | IKSDGSST |
| 184 | ARGGGSSNWYPGFFDY |
| 185 | EVQLLESGGGLVQPGGSLRLSCVASGFTFSSYAMSWVRQAPGKGLEW |
| | VSSISASGGSTYYADSVKGRFTISRDNSKNTLYLQMNSLRAEDTAVYY |
| | CGNWPEGFPAYFHYWGQGTLVTV |
| 186 | GFTFSSYA |
| 187 | ISASGGST |
| 188 | GNWPEGFPAYFHY |
| 189 | EIVMTQSPATLSVSPGERVTLSCRASQSVTSELAWYQQKPGQAPRLLIY |
| | DASTGATGIPARFSGSGSGTDFTLTISSLQSEDFAVYYCQQHNNWPLTF |
| | GGGTKVEIK |
| 190 | QSVTSE |
| 106 | DAS |
| 191 | QQHNNWPLT |
| 192 | EVQLVESGGGLVQPGGSLRLSCAASGFTFSSYWMHWVRQAPGKGLV |
| | WVSRIKSDGRSISYADSVKGRFTISRDNAKNTLYLQMNSLRAEDTAVY |
| | YCARDPHGTAAPPRDAFDIWGQGTMVTV |
| 42 | GFTFSSYW |
| 193 | IKSDGRSI |
| 194 | ARDPHGTAAPPRDAFDI |
| 196 | QSALTQPASVSGSPGQSITISCTGTSGDVGSYNLVSWYQQYPGKAPKL |
| | MIYEVDKRPSGVSNRFSGSKSGNTASLTISGLRAEDEAHYHCFSYAGSL |
| | TLVFGGGTKLTVL |
| 197 | SGDVGSYNL |
| 198 | EVD |
| 199 | FSYAGSLTLV |
| 200 | QSVLTQPPSVSGTPGQRVTISCSGSSSNIGSKTANWYQKLPGTAPKLLIY |
| | SNNQRPSGVPDRFSGSKSGTSASLAISGLQSEDEAGYYCTAWDDSLNG |
| | PVFGGGTKLTVL |
| 201 | SSNIGSK |
| 202 | SNN |
| 203 | TAWDDSLNGPV |
| 204 | EVQLVESGGGLVQPGGSLRLSCGASGFTVSGKYMTWARQAPEKGLE |
| | WVSAIYRGGGTYYADSVKGRFTISRDNSKNMLYLQMNSLRAEDTAVY |
| | YCGGSVMVSATDYWGQGTLVTV |
| 205 | GFTVSGKY |
| 206 | IYRGGGT |
| 207 | GGSVMVSATDY |
| 159 | DIQMTQSPSSLSASVGDRVTITCRASQSISSYLNWYQQKPGKAPKLLIY |
| | AASSLQSGVPSRFSGSGSGTDFTLTISSLQPEDFATYYCQQSYSTPYTFG |
| | QGTKVEIK |
| 160 | QSISSY |
| 208 | AASS |
| 162 | QQSYSTPYT |
| 209 | EVQLVESGGGLVKPGGSLRLSCAASGFTFSSYSMNWVRQAPGKGLEW |
| | VSSISSSSSYIYYADSVKGRFTISRDNAKNSLYLQMNSLRAEDTAVYYC |
| | ARDQISGYWGQGTLVTV |
| 164 | GFTFSSYS |
| 165 | ISSSSSYI |
| 210 | ARDQISGY |
| 211 | EVQLVESGGGLVQPGGSLRLSCVASGFTFSSYWMHWVRQAPGKGLV |
| | WVSRIYPDGTTTANYADSVKGRFTISRDNAKNTVYLQMNSLRAEDTA |
| | VYYCARDLRESDYWGQGTLVTV |
| 42 | GFTFSSYW |
| 212 | IYPDGTTTA |
| 213 | ARDLRESDY |
| 214 | QSALTQPASVSGSPGQSITISCTGTSSDVGNYNYVSWHQQHPGKAPKL |
| | MIYDVSNRPSGVSNRFSGSKSGNTASLTISGLQAEDEADYYCNSYTTYS |
| | THVFGTGTKVTVL |
| 215 | SSDVGNYNY |
| 47 | DVS |
| 216 | NSYTTYSTHV |
| 217 | GAGGTGCAGCTGGTGGAGTCCGGGGGAGGCTTAGTTCAGCCTGGG |
| | GGGTCCCTGAGACTCTCCTGTGCAGCCTCTGGATTCACCTTCAGTAG |
| | CTACTGGATGCACTGGGTCCGCCAAGCTCCAGGGAAGGGGCTGGTG |
| | TGGGTCTCACGTATTAATAGTGATGGGAGTAGCACAAGCTACGCGG |
| | ACTCCGTGAAGGGCCGATTCGCCATCTCCAGAGACAACGCCAAGAA |
| | CACGCTGTATCTGCAAATGAACAGTCTGAGAGCCGAGGACACGGCT |
| | GTGTATTACTGTGCAAGAGCTTTGGGTGGCTGGGATATTGACTACT |
| | GGGGCCAGGGAACCCTGGTCACCGTCTCG |
| 218 | CAGTCTGTGCTGACGCAGCCACCCTCAGCGTCTGGGACCCCCGGGC |
| | AGAGGGTCACCATCTCTTGTTCTGGAAGCAGCTCCAACATCGGAAG |
| | TAATTATGTATACTGGTACCAGCAGCTCCCAGGAACGGCCCCCAAA |
| | CTCCTCATCTATAGGAATAATCAGCGGCCCTCAGGGGTCCCTGACC |
| | GATTCTCTGGCTCCAAGTCTGGCACCTCAGCCTCCCTGGCCATCAGT |
| | GGGCTCCGGTCCGAGGATGAGGCTGATTATTACTGTGCAGCATGGG |
| | ATGACAGCCTGAGTGGTGGAGTGTTCGGCGGAGGGACCAAGCTGA |
| | CCGTCCTAGGT |
| 219 | CAGTCTGTGCTGACGCAGCCGCCCTCAGTGTCTGCGGCCCCGGGAC |
| | AGAAGGTCACCATCTCCTGCTCTGGAGGCAGCTCCTCCATTGGGAA |
| | TAATTATGTATCCTGGTACCAACTGCTCCCAGGAACAGCCCCCAAA |
| | CTCCTCATTTATGACAATAATAAGCGACCCTCAGGGATTCCTGACC |
| | GATTCTCTGGCTCCAAGTCTGGCACGTCAGCCACCCTGGGCATCAC |
| | CGGACTCCAGACTGGGGACGAGGCCGATTATTACTGCGGAACATGG |
| | GATAGCGGCCTGAGTGCTGGGGTGTTCGGCGGAGGGACCAAGCTG |
| | ACCGTCCTAGGT |
| 220 | GTGCAGCTGGTGGAGTCTGGGGGAGGCGTGGTCCAGCCTGGGAGG |
| | TCCCTGAGACTCTCCTGTGCAGCCTCTGGATTCACCTTCAGTGACTA |
| | TGCTGTGCACTGGGTCCGCCAGGCTCCAGGCAAGGGGCTGGAGTGG |
| | GCGGCACTTATATCAAATGATGGAGATTTTAAATATTACGCAGACG |
| | CCGTGAAGGGCCGATTCACCATCTCCAGAGACAATTCCAACAACAC |
| | GCTGTATCTACAAATGAACAGCCTGAGAACTGAGGACACGGCTGTG |
| | TATTACTGTGCGAGAGAAAGAGGGAGGGATAAAGTATTCCCGCCG |
| | GGCTACTGGGGCCAGGGAACCCTGGTCACCGTCTCG |
| 221 | CAGTCTGTGCTGACTCAGCCGCCCTCAGTGTCTGCGGCCCCAGGAC |
| | AGAAGGTCACCATCTCCTGCTCTGGAAGCAGCTCCAACATTGGGAA |
| | TAATTATGTATCCTGGTACCAGCAGCTCCCAGGAACAGCCCCCAAA |
| | CTCCTCATTTATGACAATAATAAGCGACCCTCAGGGATTCCTGACC |
| | GATTCTCTGGCTCCAAGTCTGGCACGTCAGCCACCCTGGGCATCAC |
| | CGGACTCCAGACTGGGGACGAGGCCGATTATTACTGCGGAACATGG |
| | GATAGCAGCCTGAGTGCTGAGGTGTTCGGCGGAGGGACCAAGCTG |
| | ACCGTCCTAGGT |
| 222 | GTGCAGCTGGTGGAGTCTGGGGGAGGCGTGGTCCAGCCTGGGAGG |
| | TCCCTGAGACTCTCCTGTGAAGCGTCTGGATTCACCTTCAGGAGTCG |
| | TGCCATGCACTGGGTCCGCCAGGCTCCAGGCAAGGGGCTGGGGTGG |
| | GTGGCAGTTATATGGTACGATGGAAGTAATAAATACTATGCAGACT |
| | CCGTGAAGGGCCGATTCACCATCTCCAGAGACAATTCCAGGAACAC |
| | GCTGTATCTGCAAATGAACAGCCTGAGAGCCGAGGACACGGCTGTA |
| | TATTACTGTGCGAGAGATTTGGGTAGTGGTTTTTCCCTTGACTACTG |
| | GGGCCAGGGAACCCTGGTCACCGTCTCG |
| 223 | CAGGCTGTGGTGACTCAGGAGCCCTCACTGACTGTGTCCCCAGGAG |
| | GGACAGTCACTCTCACCTGTGCCTCCAGCACTGGAGCTGTCACCAG |
| | TGCTCATTCTCCCCACTGGTTCCGACAGAAGCCTGGCCAAGCCCCC |
| | AGGACACTGATTTATGATACATCCAACAAACCGTCCTGGACACCTG |
| | CCCGGTTCTCAGGCTCCCTCCTTGGGGGCAAAGCTGCCCTGACCCTT |
| | TCGGGCGCGCAGCCTGAGGATGAGGCTGAGTATTACTGCTTGCTCT |
| | CCAATAGTGGAGTCCATTTTCTATTCGGCGGGGGGACCAGGTTGAC |
| | CGTCCTAGGTCA |
| 224 | GAGGTGCAGCTGGTGCAGTCTGGGGCTGAGGTGAAGAAGACTGGG |
| | TCCTCAGTGAAGCTCTCCTGCACGGCTACCGGATACACTTTCACCTA |
| | TCGCTACCTGCACTGGGTGCGACAGGCCCCCGGACAAGCACTTGAG |
| | TGGATGGGCTACATAACAATTTACAATGGTGACACCAATTACGCAC |
| | AGAAATTCCAGGACAGAGTCACCATTTCCAGGGACATGTCTCTGAG |
| | CACAGTCTACATGGAGCTGAGCAGCCTGACATCAGAGGACACGGC |
| | CATGTATTTCTGTGTAAGATCCGCATTGTATGGGGAAAATGCTTTTG |
| | ATTTTTGGGGCCAAGGGACAATGGTCACCGTCTCA |
| 225 | GAGGTGCAGCTGGTGGAGTCGGGGGGAGGCTCAGTTCAGCCGGGG |
| | GGGTCCCTGAGACTCTCCTGTGCAGCCTCTGGATTCACCTTCAGTAA |
| | CTACTGGATGCACTGGGTCCGCCAAGCTCCAGGGAAGGGGCTGGTG |
| | TGGGTCTCACGTCTTAATATTGATGGGAGTAACACATTCTACGCGG |
| | ACTCCGTCAAGGGCCGATTCACCATCTCCAGAGACAACGCCAAGAA |
| | CACGCTGTATCTGCAAATGAACAGTCTGAGAGCCGAGGACACGGCT |
| | GTGTATTACTGTGTAAGAGGAGGGAGTGACTCCGGTGACTCCGTTC |
| | CCTTCGATCTCTGGGGCCGTGGCACCCTGGTCACCGTCTCG |
| 226 | GAAATAGTGATGACGCAGTCTCCAGCCACCCTGTCTGTGTCTCCAG |
| | GGGAAAGGGCCACCCTCTCCTGCAGGGCCAGTCAGAGTGTTAGCAG |
| | CAACTTAGCCTGGTACCAGCAGAACCCTGGCCAGGCTCCCAGGCTC |
| | CTCATCTATGGTGCATCCACCAGGGCCACTGGTATCCCAGCCAGGT |
| | TCAGTGGCAGTGGATCTGGGACAGAGTTCACTCTCACCATCAGCAG |
| | CCTGCAGTCTGAAGATTTTGCAGTTTATTACTGTCAGCACTATAATA |
| | ACTGGCCGTTCACTTTTGGCCAGGGGACCAAGGTGGAAATCAAAC |
| 227 | GAGGTGCAGCTGGTGGAGTCCGGGGGAGGCTTAGTTCAGCCTGGG |
| | GGGTCCCTGAGACTCTCCTGTGCAGCCTCTGGATTCACCTTCAGTAG |
| | CTACTGGATGCACTGGGTCCGCCAAGCTCCAGGGAAGGGGCTGGTG |
| | TGGGTCTCACGTATTAATAATGATGGGAGTAGCACAGGCTACGCGG |
| | ACTCTGTGGAGGGCCGATTCACCATCTCCAGAGACAACGCCAAGAA |
| | CACGCTGTATCTGCAAATCAACAGTCTGAGAGCCGAGGACACGGCT |
| | GTGTATTACTGTGCAAGACAAGATGGTCACTACTACTACAGTATGG |
| | ACGTCTGGGGCCAAGGGACCACGGTCACCGTCTC |
| 228 | CAGTCTGCCCTGACTCAGCCTCGCTCAGTGTCCGGGTCTCCTGGACA |
| | GTCAGTCACCATCTCCTGCACTGGAACCAGCAGTGATGTTGGTGGT |
| | TATAACTATGTCTCCTGGTACCAACAGCACCCAGGCAAAGCCCCCA |
| | AACTCATGATTTATGATGTCAGTGAGCGGCCCTCAGGGGTCCCTGC |
| | TCGCTTCTCTGGCTCCAAGTCTGGCAACACGGCCTCCCTGACCATCT |
| | CTGGGCTCCAGGCTGAGGATGAGGCTGATTATTACTGCTGCTCATA |
| | TGCAGGCAACTTCCGTTGGGTGTTCGGCGGAGGGACCAAGCTGACC |
| | GTCCTAGG |
| 229 | GAGGTGCAGCTGGTGGAGTCTGGGGGAGGCTTGGTCCAGCCGGGG |
| | GGGTCCCTGAAACTCTCCTGTGCAGCCTCTGGGTTCAGTTTCAGTGG |
| | CGCTGCGATGCACTGGGTCCGCCAGTCCTCCGGGAGAGGGCTTGAG |
| | TGGCTTGGCCGTATTAGAAGCAAAACTAACGACTATGCGACAGCAT |
| | ATGCAGAGTCGCTGCACGGCAGGTTCACCATCTCCAGAGATGATGC |
| | AAAGAACACGGCGTATCTACAAATGAACAGGCTGAAAAGCGAGGA |
| | CACGGCCATATATTATTGTACAACCGTCTTGAGTAAGGGAGATCAT |
| | GCGGTCTGGTTGGGCCCCTGGGGCCCGGGAACCCTGGTCACCGTCT |
| | C |
| 230 | CAGCCTGTGCTGACTCAGCCACCTTCCTCCTCCGCGTCTCCTGGAGA |
| | ATCCGCCAGACTCACCTGCACCTTGCCCAGTGACATCAGTGTTGCT |
| | GCGTCTGACATTTATTGGTATCAACAGAAGGCAGGGAGCCCTCCCA |
| | ACTTTCTCCTCTACGACCCGTCAGACTCACATAAGGGCCAGGACTC |
| | TGGAGTCCCCAGCCGCTTCTCTGGATCCAGAGATGGATCAGCCAAT |
| | TCAGGGTTTTTACTGATTTCCGGGGTCCAGTCTGAGGATGAGGCTG |
| | ACTATTACTGCATGGTCTGGCCACCCAATACTGTGGGTGTCGTCTTC |
| | GGCGGAGGGACCACTCTGACCGTC |
| 231 | CAGGCTGTGGTGACTCAGGAGCCCTCACTGACTGTGTCCCCAGGAG |
| | GGACAGTCACTCTCACCTGTGCTTCCAGCACTGGAGCAGTCACCAG |
| | TGGTTACTATCCAAACTGGTTCCAGCAGAAACCTGGACAAGCACCC |
| | AGGGCACTGATTTATAGTACAAGCAACAAACACTCCTGGACCCCTG |
| | CCCGGTTCTCAGGCTCCCTCCTTGGGGGCAAAGCTGCCCTGACACT |
| | GTCAGGTGTGCAGCCTGAGGACGAGGCTGAGTATTACTGCCTGCTC |
| | TTCTATGGTGGTGTTCATGTCTTCGGAGCTGGGACCAAGGTCACCGT |
| | CCTAGGT |
| 232 | GAGGTGCAGCTGGTGGAGTCCGGGGGAGACTTAGTTCAGCCGGGG |
| | GGGTCCCTAAGACTCTCCTGTGCAGCCTCTGGATTCACCTTCAGTAC |
| | CTACTGGATGCACTGGGTCCGCCAAGCTCCCGGGCAGGGGCTGGTG |
| | TGGGTCTCACGTATTAATGGTAATGGGAGAATCACAAACTACGCGG |
| | ACTCCGTGAAGGGCCGATTCACCGTCTCCCGAGACAACGCCAAGAA |
| | CACGGTGGATCTGCAAATGAACAGTCTGAGAGCCGAGGACACGGC |
| | TGTGTATTACTGTGCAAGAATCAATGAATGGGGAGACGTCTGGGGC |
| | AAAGGGACCACGGTCACCGTCTCG |
| 233 | GATATTGTGATGACCCAGACCCCACTCTCTCTGTCCGTCACCCCTGG |
| | ACAGCCGGCCTCCATCTCCTGCAAGTCTAGTCAGAGCCTCCTGCAT |
| | AATGATGGAAAGACCTTTTTGCATTGGTACCTGCAGAAGCCAGGCC |
| | AGTCTCCACAGCTCCTAATCTATGAAGTTTCCAGCCGCTTCTCTGGA |
| | GTGCCAGATAGGTTCAGTGGCAGCGGGTCAGGGACAGATTTCACAC |
| | TGAAAATCAGCCGGGTGGAGGCTGAGGATGTTGGAGTTTATTACTG |
| | CATGCAAGGTATACACCTTCCTCCCACTTTCGGCGGGGGGACCAAG |
| | GTGGAAATCAAACGT |
| 234 | GAGGTGCAGCTGGTGGAGTCCGGGGGAGGCTTAGTTCAGCCGGGG |
| | GGGTCCCTGAGACTCTCCTGTGCAGCCTCTGGATTCACATTCAGTAG |
| | TTACTGGATGGAATGGGTCCGCCAAGCTCCAGGGAAGGGGCTGGTT |
| | TGGGTCTCACATATTAGTAGTGATGGGAGTGTTACAAGGTACGTGG |
| | ACTCCGTGAAGGGCCGATTCACCATCTCCAGAGACAACGCCAAGAA |
| | CACGCTGTATCTGCAAATGAATAGTCTGAGAGCCGAGGACACGGGT |
| | GTATATTATTGTGCAAAAGATCTTCACTGGAACGCTCTTGATGTGTG |
| | GGGCCAAGGGACAATGGTCACCGTCTCG |
| 235 | GAAATAGTGATGACGCAGTCTCCAGCCACCCTGTCTGTGTCTCCAG |
| | GGGAAAGAGCCACCCTCTCCTGCAGGGCCAGTCAGAGTGTTAGCAG |
| | CAACTTAGCCTGGTACCAGCAGAAACCTGGCCAGGCTCCCAGGCTC |
| | CTCATCTATGGTGCATCCACCAGGGCCACTGGTATCCCAGCCAGGT |
| | TCAGTGGCAGTGGGTCTGGGACAGAGTTCACTCTCACCATCAGCAG |
| | CCTGCAGTCTGAAGATTTTGCAGTTTATTACTGTCAGCAGTATAATA |
| | ACTGGCCTCCGTGGACGTTCGGCCAAGGGACCAAGGTGGAAATCA |
| | AACGT |
| 236 | GAGGTGCAGCTGGTGGAGTCCGGGGGAGGCTTAGTTCAGCCTGGG |
| | GGGTCCCTGAGACTCTCCTGTGCAGCCTCTGGATTCACCTTCAGTAG |
| | CTACTGGATGCACTGGGTCCGCCAAGCTCCAGGGAAGGGGCTGGTG |
| | TGGGTCTCACGTATTAATAGTGATGGGAGTAGCACAAGCTACGCGG |
| | ACTCCGTGAAGGGCCGATTCACCATCTCCAGAGACAACGCCAAGAA |
| | CACGCTGTATCTGCAAATGAACAGTCTGAGAGCCGAGGACACGGCT |
| | GTGTATTACTGTGCAAGAGGAGTCCGCAGTGGCTGGTACGCTGATG |
| | CTTTTGATATCTGGGGCCAAGGGACAATGGTCACCGTCTCG |
| 237 | GAGGTGCAGCTGGTGGAGTCCGGGGGAGGCTTAGTTCAGCCGGGG |
| | GGGTCCCTGGGACTCTCCTGTGCAGCCTCTGGATTCACCTTCAGTAA |
| | CTACTGGATGCACTGGGTCCGCCAAGCTCCAGGGAAGGGGCTGGTG |
| | TGGGTCTCGCGTATTAATAGTGATGGGAGTGACACAAGCTACGCGG |
| | ACTCCGTGAAGGGCCGATTCACCATCTCCAGAGACAACGCCGAGAA |
| | CACGCTGTATCTGCACATGAGCAGTCTGAGAGCCGAGGACACGGCT |
| | GTCTATTACTGTGCAAGGGAATTGGGCTACGGTGACTACGGTATGG |
| | ACGTCTGGGGCAAAGGGACCACGGTCACCGTCTCG |
| 238 | CAGTCTGTGCTGACGCAGCCACCCTCAGCGTCTGGGACCCCCGGGC |
| | AGAGGGTCACCATCTCTTGTTCTGGAAGCAGCTCCAACATCGGAAG |
| | TCATTCTGTGATCTGGTACCAGCAGCTCCCAGGAACGGCCCCCAAA |
| | CTCCTCGTCTATAGTGATAATCAGCGGCCCTCAGGGGTCCCTGACC |
| | GATTCTCTGGCTCCAAGTCTGGCACCTCAGCCTCCCTGGCCATCAGT |
| | GGGCTCCAGTCTGAGGATGAGGCTGATTATTACTGTGCAGCATGGG |
| | ATGACAGCCTGAATCATCTTCATGTGGTATTCGGCGGAGGGACCAA |
| | GCTGACCGTCCTA |
| 239 | GAGGTGCAGCTGGTGGAGTCCGGGGGAGGCTTAGTTCAGCCTGGG |
| | GGGTCCCTGAGACTCTCCTGTGCAGCCTCCGGATTCATCTTCAGTAA |
| | CTACTGGATGCACTGGGTTCGCCAAGCTCCAGGGGAGGGGCTGGTG |
| | TGGGTCTCACGTATTAATAAAGATGGGAGTAGCACATTTTACGCGG |
| | ACTCCGTGAAGGGCCGATTCACCATCTCCAGAGACAACGCCAAGAA |
| | CACGCTGTATCTGCAAATGAACAGTCTGAGAGCCGAGGACACGGCT |
| | GTGTATTATTGTACAAGAGATTTCGATTTTTGGAGTGGCTACTGGGG |
| | CCAGGGAACCCTGGTCACCGTCTCG |
| 240 | CAGTCTGCCCTGACTCAGCCTGCCTCCGTGTCTGGGTCTCCTGGACA |
| | GTCGATCACCATCTCCTGCACTGGAACCAGCAGTGACGTTGGTGGT |
| | TGTGAGTATGTCTCCTGGTACCAACAGCACCCAGGCAAAGTCCCCA |
| | AACTCATCATTTATGAGGTCAGTAATCGGCCCTCAGGGGTTTCTAAT |
| | CGCTTCTCTGGCTCCAAGTCTGGCAACACGGCCTCCCTGACCGTCTC |
| | TGGGCTCCAGGCTGAGGACGAGGCTGATTATTTCTGCAGCTCCTGT |
| | ACAACCAGCGGCTCTTATGTCTTCGGAGCTGGGACCAAGGTCACCG |
| | TCGTA |
| 241 | GAGGTGCAGCTGGTGGAGTCCGGGGGAGGCGTAGTTCAGCCTGGG |
| | GAGTCCCTGAGACTCTCCTGTGCAGCCTCCGGAGTCACCTTCAGTA |
| | GTTACTGGATGCATTGGGTCCGCCAAGTTCCAGGGAAGGGGCTGGT |
| | GTGGGTCGCACGTATTAATATTGATGGGACCAGTACAACCTACGCG |
| | GACTGTGTGAAGGGCCGATTCACCATCTCCAGAGACAACGCCAAGA |
| | ACACGCTGTATCTGCAAATGAGCAGTCTGAGAGCCGAGGACACGG |
| | CTGTTTATTACTGTGCAACAGAGCGAGGATTACTTAGTGGTGGTCG |
| | CTGGCACTCCTCCCACTTTGACTACTGGGGCCAGGGAACCCTGGTC |
| | ACCGTCTCG |
| 242 | GACATCCAGATGACCCAGTCTCCTTCCACCCTGTCTGCATCTGTAGG |
| | AGACAGAGTCACCATCACTTGCCGGGCCAGTCAGAGTATTAGTAGC |
| | TGGTTGGCCTGGTATCAGCAGAAACCAGGGAAAGCCCCTAAGGTCC |
| | TGATCTATAAGACGTCTAGTTTAGAAAGTGGGGTCCCATCAAGGTT |
| | CAGCGGCAGTGGCTCTGGGACAGAATTCACTCTCACCATCAGCAGC |
| | CTGCAGCCTGATGATTTTGCAACTTATTACTGCCAACAGTATGACA |
| | GTTCTTCTCTCACGTGGACGTTCGGCCAAGGGACCAAGGTGGAAAT |
| | CAAAC |
| 243 | GAGGTGCAGCTGGTGGAGTCCGGGGGAGGCTTAGTTCAGCCTGGG |
| | GGGTCCCTGAGACTCTCCTGTGCAGCCTCTGGATTCACCTTCAGTAG |
| | GTTCTGGATGCACTGGGTCCGCCAGGTTCCAGGGAAGGGGCTGGTG |
| | TGGATCTCACGTATTAATAGTGATGCGACTAGCTCAAGCTACGCGG |
| | ACTCCGTGAAGGGCCGATTCACCATCTCCAGAGACAACGCCAAGAA |
| | CACACTGTTTCTGCAAATGAACAGTCTGAGAGCCGAGGACACGGCT |
| | GTCTATTACTGTGCAACCAGCAATACAGCTCTGGTTTATTTGCCTGA |
| | GAACTGGGGCCAGGGAACCCTGGTCACCGTCTCG |
| 244 | GAAATTGTGTTGACACAGTCTCCAGCCACCCTGTCTTTGTCTCCAGG |
| | AGAGAGAGCCACCCTCTCCTGCAGGGCCAGTCAGAGTGTTAGCGAC |
| | TACTTAGCCTGGTACCAACAGAAACCTGGCCAGGCTCCCAGGCTCC |
| | TCATCTATGATGCATCCAACAGGGCCACTGGCATCCCAGCCAGGTT |
| | CAGTGGCGGTGGGTCTGGGACAGACTTCACTCTCACCATCAGCAGC |
| | CTAGAGCCTGAAGATTTTGCAGTTTATTACTGTCAGCAGCGCAGCT |
| | ACTGGCCTCCGACGTTCGGCCAAGGGACCAAGGTGGAAATCAAAC |
| 245 | CAGTCTGCCCTGACTCAGCCTCCCTCCGCGTCCGGGTCTCCTGGACA |
| | GTCAGTCACCATCACCTGCACTGGAACCAGCAGTGACGTTGGTGGT |
| | TATAACTATGTCTCCTGGTACCAACAGCACCCAGGCAAAGCCCCCA |
| | AACTCATGATTTATGAGGTCAGTAAGCGGCCCTCAGGGGTCCCTGA |
| | TCGCTTCTCTGGCTCCAAGTCTGGCAACACGGCCTCCCTGACCGTCT |
| | CTGGGCTCCAGGCTGAGGATGAGGCTGATTATCACTGCAGCTCATT |
| | TGCAGGCGACAACAATTCCCCGGTATTCGGCGGAGGGACCAAATTG |
| | ACCGTCCTAG |
| 246 | GAGGTGCAGCTGGTGGAGTCCGGGGGAGGCTTAATTCACCCGGGG |
| | GGGTCCCTGAGACTCTCCTGTGCAGCCTCTGGATTCACCTTCAGTAG |
| | CTACTGGATGCACTGGGTCCGCCAAGCCCCAGGGAAGGGGCTGGTG |
| | TGGGTCTCACATATTAAGAGTGATGGGAGTAACACAATCTACGCGG |
| | ACTCCGTGAAGGGCCGATTCACCATCTCCAGAGACAACGCCAAGAA |
| | CACGCTGTATCTACAAATGAACAGTCTGAGAGCCGAGGACACGGCT |
| | GTGTATTACTGTGGAAGAGGGCGCAGTTTGACCCCACGCTCGGCCA |
| | TTGACTACTGGGGCCAGGGAACCCTGGTCACCGTCTC |
| 247 | CTGGTGGAGTCTGGGGGAGGCTTGGTCCAGCCTGGGGGGTCCCTGA |
| | GACTCTCCTGTGCAGCCTCTGGATTCACCTTCAGCAGTTATGCTATG |
| | CACTGGGTCCGCCAGGCTCCAGGGAAGGGACTGGAGTTTGCTTCAG |
| | CTATTAGTAGTGATGGGGGTACCACATATTACGCAAACTCTGTGAA |
| | GGGCAGATTCACCATCTCCAGAGACAATTCCAAGAACACGCTGTAT |
| | CTTCAAATGGGCAGCCTGAGGGATGAAGACATGGCTGTGTATTACT |
| | GTGCGAGAGATGATCTGAGCACCAGCTGGGACCTTGACTACTGGGG |
| | CCAGGGAACCCTGGTCACCGTCTC |
| 248 | CAGTCTGCCCTGACTCAGCCTGCCTCCGTGTCTGGGTCTCCTGGACA |
| | GTCGATCACCATCTCCTGCACTGGAACCAGCAGTGACGTTGGTGGT |
| | TATAACTATGTCTCCTGGTACCAACAGCACCCAGGCAAAGCCCCCA |
| | AACTCATGATTTATGACGTCAGTAATCGGCCCTCAGGGGTTTCTAAT |
| | CGCTTCTCTGGCTCCAAGTCTGGCAACACGGCCTCCCTGACCATCTC |
| | TGGGCTCCAGGCTGAGGACGAGGCTGATTATTACTGCAGCTCATAT |
| | ACAATCAGCAGCACTAACGTCTTCGGAACTGGGACCAAGGTCACCG |
| | TCCTAG |
| 249 | CAGTCTGCCCTGACTCAGCCTGCCTCCATGTCTGGGTCTCCTGGACA |
| | GTCGATCACCATCTCCTGCACTGGAACCAGCAGTGATGTTGGAGCT |
| | CATAACTTTATCTCCTGGTACCAACAACACCCAGGCAAAGCCCCCA |
| | AACTCATGATTTTTGAGGTCAATAAGCGGCCCTCAGGGGTTTCTAA |
| | TCGCTTCTCTGGCTCCAAGTCTGGCAACACGGCCTCCCTGACAATCT |
| | CTGGCCTCCAGGCTGAGGACGAGGCTGATTATTTCTGCTGCTCATAT |
| | GCAGGTTATAGCACTTGGGTGTTCGGCGGAGGGACCAAGCTGACCG |
| | TCCTAG |
| 250 | GAGGTGCAGCTGGTGGAGTCCGGGGGAGGCGTAGTCCAGCCTGGG |
| | GGGTCCCTGAGACTCTCCTGTGAAGTCTCTGGATTCATCTTCAGTAG |
| | CTACTGGATGCACTGGGTCCGCCAAGTTGCAGGGAAGGGGCTGGTG |
| | TGGGTCTCACGTATAAATAGTGATGGGAGTAGTACAAGTTACGCGG |
| | ACTCCGTGAAGGGCCGATTCACCATCTCCAGAGACAACGCCAAGAA |
| | TACGCTGAATCTGCAAATGAATTTTCTGAGAGCCGAGGACACGGCT |
| | GTGTATTATTGTGCAAGAGGGGGTGATCACGGTGACTACGGCTTCT |
| | TTGAGTCTTGGGGCCAGGGAACCCTGGTCACCGTCTC |
| 251 | GAGGTGCAGCTGGTGGAGTCCGGGGGAGGGTTAGTTCAGCCTGGG |
| | GGGTCCCTGAGACTCTCCTGTGCAGCCTCTGGATTCACCTTCAGTAA |
| | TCACTGGATGCACTGGGTCCGCCAAGCTCCAGGGAAGGGGCTGCTG |
| | TGGGTCTCGCGTATTGATACTGGTGGGAGTACCACAAACTACGCGG |
| | ACTCCGTGAAGGGCCGATTCACCATCTCCAGAGACAGCGCCAAGAA |
| | CACGGTGTATCTACAAATGAACAGTCTGAGAGCCGAAGACACGGCT |
| | GTTTATTACTGTGCAAGAGGCGGTCTCTTCTACTACGGTATGGACGT |
| | CTGGGGCCAAGGGACCACGGTCACCGTCTC |
| 252 | GAAATTGTGTTGACGCAGTCTCCAGGCACCCTGTCTTTGTCTCCAGG |
| | GGAAAGGGCCACCCTCTCCTGCAGGGCCAGTCAGACTAGTAGCAGC |
| | ACCTCCTTAGCCTGGTATCAGCAGAAACGTGGCCAGGCTCCCAGGC |
| | TCCTCATCTATGGTGCATCCCGCAGGGCCACTGGCATCCCAGACAG |
| | GTTCAGTGGCAGTGGGTCTGGGACAGACTTCACTCTCACCATCAGC |
| | AGACTGGAGCCTGAAGATTTTGCAGTATATTACTGTCAGCAATTTA |
| | GTGGCTCACCTGCGTACACTTTTGGCCAGGGGACCAAGGTGGAAAT |
| | CAAAC |
| 253 | GATATTGTGATGACCCAGACTCCACTCTCCCTGCCCGTCACCCCTGG |
| | AGAGCCGGCCTCCATCTCCTGTAGGTCTAGTCAGAGCCTCTTGGAT |
| | AGTGCTGATGGAAACACCTATTTGGACTGGTACCTGCAGAAGCCAG |
| | GGCAGTCTCCACAGCTCCTGATCCATACGCTTTCCCATCGGGCCTCT |
| | GGAGTCCCAGACAGGTTCAGTGGCAGTGGGTCAGGCACTGATTTCA |
| | CACTGAAAATCAGCAGGGTGGAGGCTGAAGATGTTGGAGTTTATTA |
| | CTGCATGCAACGTATAGCCTTTCCCGTCACTTTCGGCCCTGGGACCA |
| | AAGTGGATATCAAAC |
| 254 | GAGGTGCAGCTGGTGGAGTCCGGGGGAGGCTTAGTTCAGCCTGGG |
| | GGGTCCCTGAGACTCTCCTGTGCAGCCTCTGGATTCACCTTCAGTAA |
| | CTACTGGATGCACTGGGTCCGCCAAGCTCCAGGGGAGGGGCTGGA |
| | GTGGGTCTCACGTATTAATAGTGATGGGAATAAGGTAAGTTACGCG |
| | GACTCCGTGAGGGGCCGATTCACCATCTCCAGAGACAACGCCAAGA |
| | ACACGCTGTATCTACAAATGAACAGCCTGACAGGCGAGGACACGG |
| | CTGTGTATTATTGTGCAAGATCTAACTGGGGATCGGCAGACTACTG |
| | GGGCCAGGGAACCCTGGTCACCGTCTC |
| 255 | GAGGTGCAGCTGGTGGAGTCTGGGGGAGGCTCAGTTCAGCCTGGG |
| | GGGTCCCTGAGACTCTCCTGTGCTGCCTCTGGATTCACCTTCAGTAG |
| | ATACTGGATGCACTGGGTCCGCCAGGCTCCAGGGGAGGGGCTCGTT |
| | TGGGTCTCACGTATAAATAGTGATGAGACTGACAAGCTTTACGCGG |
| | ACTCCGTGAAGGGCCGATTCTCCATCTTCAGAGACAACGCCAAGAA |
| | CACACTATATCTGCAAATGAACAGACTGAGAGCCGAGGACACGGC |
| | TGTATACTACTGTGCAAGAGATCGAGAGGATGTTGTAGTGGGGCCA |
| | GCTACTCAACACACCATCTTTAACTCCTGGGGCCAGGGAACCCTGG |
| | TCACCGTCTC |
| 256 | CAGTCTGCCCTGACTCAGCCTGCCTCCGTGTCTGGGTCTCCTGGACA |
| | GTCGATCACCATCTCCTGCACTGGAACCAGCAGTGATGTTGGGAGT |
| | TCTAACTTTGTCTCCTGGTACCAACAACACCCAGGCAAAGCCCCCA |
| | AACTCATCCTTTATGAGGTCAGTAAGCGGCCCGCTGGAGTTTCTAG |
| | TCGCTTCTCTGGCTCCAGGTCTGGCAACACGGCCTCCCTGACAATCT |
| | CTGGAGCCCAGGCTGAGGACGAGGCTGACTATTCCTGCTGCTCAAC |
| | TTCTTCTGTTGGCACTCTTTATGTCTTCGGAACTGGGACCAAGGTAA |
| | CCGTCCTAG |
| 257 | CAGTCTGTGCTGACGCAGCCGCCCTCAGTGTCTGCGGCCCCAGGAC |
| | AGAAGGTCACCATCTCCTGCTCTGGAAGCAACTACAACATTGGGAA |
| | TAATTATGTATCCTGGTACCAGCGACTCCCAGGAACAGCCCCCAAA |
| | CTCCTCATTTATGACAATAATAAGCGACCCTCAGGGATTCCTGACC |
| | GATTCTCTGGCTCCAAGTCTGGCACGTCAGCCACCCTGGGCATCAC |
| | CGGACTCCAGACTGGGGACGAGGCCGATTATTACTGCGGAACATGG |
| | GATAGCAGCCTGAGGGCTGGGGTGTTCGGCGGAGGGACCAAGCTG |
| | ACCGTCCTAG |
| 258 | GAGGTGCAGCTGGTGGAGTCCGGGGGAGGCTTAGTTCAGCCTGGG |
| | GGGTCCCTGAGACTCTCCTGTGCAGCCTCTGGATTCACTTTCAGTAG |
| | GCACTGGATGCACTGGGTCCGCCAAGCTCCAGGGAAGGGGCTGGT |
| | GTGGGTCTCACGTATTAATAGTGATGGGAGTAGCACAAGTAACGCG |
| | GACTCCGTGAAGGGCCGAATCACCATCTCCAGAGACAACGGCAAG |
| | AACACGCTGTATCTGCAGATGAACAGTCTGAGAGCCGAGGACACG |
| | GCTGTGTATTACTGTGCAAGGGAGATAGCATCGGGGACAGATGCTT |
| | TTGATATCTGGGGCCAAGGGACAATGGTCACCGTCTC |
| 259 | GAGGTGCAGCTGGTGGAGTCTGGGGGAGGCTTAGTAAAGCCTGGG |
| | GAGTCCCTTAGACTCTCCTGTGTAGGTTCTGGATTCACTCTCACTAA |
| | CGCCTGGATGATCTGGGTCCGCCAGACTTCAGGGAAGGGGCTGGAA |
| | TGGGTTGGTCGCATCAAGAGTAAAATTGATGGTGGGGCAATCGACT |
| | ACGGTGCACCCGTGAAAGGTAGATTTACCATCTCAAGAGATGATAC |
| | AAAAAACACGGTGTATCTGCAAATGAACAGCCTGCAAACCGACGA |
| | CACAGGCGTCTATTTCTGTACCACAGATCGTTATAGTACTGGCTACT |
| | ACGGCATGGACGACTACTGGGGCCAGGGAACCCTGGTCACCGTCTC |
| 260 | TCCTATGAGCTGACTCAGCCACCCTCAGTGTCCGTGTCCCCAGGAC |
| | AGACAGCCACCATCACCTGCTCTGGAGATAAATTGGGAGATAAATA |
| | TTCTTTCTGGTATCAACAGAAGCCAGGCCAGTCCCCTGTGGTGGTC |
| | ATCTATCAAGATTCCAAGCGGCCCTCAGGGATCCCTGAGCGATTCT |
| | CTGGCTCCAACTCTGGGAACACAGCCACTCTGACCATCAGCGGGAC |
| | CCAGGCTATGGATGAGGCTGACTATTACTGTCAGGTGTGGGACACC |
| | GACTCTGCAGTCTTCGGAACTGGGACCAGGGTCACCGTCCT |
| 261 | GACATCCAGATGACCCAGTCTCCATCCTCCCTGTCTGCATCTGTAGG |
| | AGACAGAGTCACCATCACTTGCCGGGCAAGTCAGAGCATTAGCAGC |
| | TATTTAAATTGGTATCAGCAGAAACCAGGGAAAGCCCCTAAGCTCC |
| | TGATCTATGCTGCATCCAGTTTGCAAAGTGGGGTCCCATCAAGGTT |
| | CAGTGGCAGTGGATCTGGGACAGATTTCACTCTCACCATCAGCAGT |
| | CTGCAACCTGAAGATTTTGCAACTTACTACTGTCAACAGAGTTACA |
| | GTACCCCGTACACTTTTGGCCAGGGGACCAAGGTGGAAATCAAAC |
| 262 | GAGGTGCAGCTGGTGGAGTCTGGGGGAGGCCTGGTCAAGCCTGGG |
| | GGGTCCCTGAGACTCTCCTGTGCGGCCTCTGGATTCACCTTCAGTAG |
| | CTATAGCATGAACTGGGTCCGCCAGGCTCCAGGGAAGGGGCTGGA |
| | GTGGGTCTCATCCATTAGTAGTAGTAGTAGTTACATATACTACGCA |
| | GACTCAGTGAAGGGCCGATTCACCATCTCCAGAGACAACGCCAAG |
| | AACTCACTGTATCTGCAAATGAACAGCCTGAGAGCCGAGGACACG |
| | GCTGTGTATTACTGTGCGAGAGATACGATTTCTCGATACTGGGGCC |
| | AGGGAACCCTGGTCACCGTCTC |
| 263 | GAGGTGCAGCTGGTGGAGTCTGGGGGAGGCCTGGTCAAGCCTGGG |
| | GGGTCCCTGAGACTTTCCTGTGCAGCCTCTGGATTCACCTTCAGTAG |
| | TTACAGCATGAACTGGGTCCGCCAGGCTCCAGGGAAGGGGCTGGA |
| | GTGGGTCTCATCCATTAGTGGTAGTAGTAGTTACATGCACTACGCA |
| | GAGTCAGTGAAGGGCCGATTCACCATCTCCAGAGACAACGCCAAG |
| | AACTCACTGTATCTGCAAATGAATAGCCTGAGAGCCGAGGACACGG |
| | CTGTGTATTACTGTGCGAGTACGGGGATTATCACGTATTACTATGCT |
| | TCGGGGGTCCCTGACTACTGGGGCCAGGGAACCCTGGTCACCGTCT |
| | C |
| 264 | CAGTCTGTGCTGACTCAGCCACCCTCAGCGTCTGGGACCCCCGGGC |
| | AGAGGGTCACCATGTCTTGTTCTGGAAGCAGCTCCAACATCGGAAG |
| | AAATTATGTATACTGGTACCAGCAGCTCCCAGGAACGGCCCCCAAA |
| | CTCCTCATCTATAGGAATAATCAGCGACCCTCAGGGGTCCCTGACC |
| | GATTCTCTGGCTCCAAGTCTGGCACCTCAGTCTCCCTGGCCATCAGT |
| | GGGCTCCGGTCCGAGGATGAGGCTGATTATTACTGTGCAGCATGGG |
| | ATGACAGCCTGAGTGGTGTGGTATTCGGCGGAGGGACCAAGCTGAC |
| | CGTCCTAG |
| 265 | GAAATTGTGTTGACGCAGTCTCCAGGCACCCTGTCTTTGTCTCCAGG |
| | GGAAAGAGCCACCCTCTCCTGCAGGGCCAGTCAGAGTGTTAGCAGC |
| | AACTTAGCCTGGTACCAGCAGAAACCTGGCCAGGCTCCCAGGGTCC |
| | TCATCTATGGTGCATCCAGCAGGGCCACTGGCATCCCAGACAGGTT |
| | CAGTGGCAGTCGGTCTGGGACAGACTTCACTCTCACCATCAGCAGA |
| | CTGGAGCCTGAAGATTTTGCAGTGTATTACTGTCAGCGGTATGATA |
| | ACTCACCTCGGACGTTCGGCCAAGGGACCAAGGTGGAAATCAAAC |
| 266 | GAGGTGCAGCTGGTGGAGTCTGGGGGAGGCTTGGTAAAGCCTGGG |
| | GGGTCCCTTAGACTCTCCTGTGCAGCCTCTGGTTTCACTTTCATTAA |
| | CGCCTGGATGAACTGGGTCCGCCAGGCTCCAGGGAAGGGGCTGGA |
| | GTGGGTCGGCCTAATTAAAAGCAAAACTGATGGTGGGACAATAGA |
| | CTACGCTGCACCCGTGAAAGGCAGATTCACTATTTCAAGAGATGAT |
| | TCAGAAAAAATGTTGTATCTGCAAATGGACAGCCTGAAGACCGAG |
| | GACACAGCCGTGTATTACTGTACCACAATGTATGGCTGGAAGGACG |
| | CGAGGGACTATTGGGGCCAGGGAACCCTGGTCACCGTCTC |
| 267 | GAAATTGTGTTGACGCAGTCTCCAGGCACCCTGTCTTTGTCTCCAGG |
| | GGAAAGAGCCACCCTCTCCTGCAGGGCCAGTCAGAGTGTTAGCAGC |
| | AGCTACTTAGCCTGGTACCAGCAGAAACCTGGCCAGGCTCCCAGGT |
| | TCCTCATCTATGGTGCGTCCAGTAGGGCCACTGGCATCCCGGACAG |
| | GTTCAGTGGCAGTGGGTCTGGGACAGACTTCACTCTCACCATCAGC |
| | AGACTGGAGCCTGAAGATTTTGCAGTGTATTACTGTCAGCAGTATG |
| | GTAGCTCACCGTACACTTTTGGCCAGGGGACCAAGGTGGAAATCAA |
| | AC |
| 268 | GAGGTGCAGCTGGTGGAGTCCGGGGGAGGCTTAGTTCAGCCTGGG |
| | GGGTCCCTGAGACTCTCCTGTGCAGCCTCTGGATTCACCTTCAGTAG |
| | CTACTGGATGCACTGGGTCCGCCAAGCTCCAGGGAAGGGGCTGGTG |
| | TGGGTCTCACGTATTAAAAGTGATGGGAGTAGCACAAGCTACGCGG |
| | ACTCCGTGAAGGGCCGATTCACCATCTCCAGAGACAACGCCAAGAA |
| | CACGCTGTATCTGCAAATGAACAGTCTGAGAGCCGAGGACACGGCT |
| | GTGTATTACTGTGCAAGAGGGGGTGGCAGCAGCAACTGGTACCCGG |
| | GTTTCTTTGACTACTGGGGCCAGGGAACCCTGGTCACCGTCTC |
| 269 | GAGGTGCAGCTGTTGGAGTCTGGGGGAGGCTTGGTACAGCCGGGG |
| | GGGTCCCTGAGACTCTCCTGTGTAGCCTCTGGATTCACCTTCAGCAG |
| | CTATGCCATGAGCTGGGTCCGCCAGGCTCCAGGGAAGGGGCTGGA |
| | GTGGGTCTCTAGTATTAGTGCTAGTGGTGGTAGCACATATTACGCA |
| | GACTCCGTGAAGGGCCGGTTCACCATCTCCAGAGACAATTCCAAGA |
| | ACACGCTGTATCTGCAAATGAACAGCCTGAGAGCCGAGGACACGG |
| | CCGTATATTACTGTGGGAATTGGCCGGAAGGATTCCCGGCCTACTT |
| | TCACTACTGGGGCCAGGGAACCCTGGTCACCGTCTC |
| 270 | GAAATAGTGATGACGCAGTCTCCAGCCACCCTGTCTGTGTCTCCAG |
| | GGGAAAGAGTCACCCTCTCCTGCAGGGCCAGTCAGAGTGTTACCAG |
| | CGAGTTGGCCTGGTACCAGCAGAAACCTGGCCAGGCTCCCAGGCTC |
| | CTCATCTATGATGCATCCACCGGGGCCACTGGTATCCCAGCCAGGT |
| | TCAGTGGCAGTGGGTCTGGGACAGACTTCACTCTCACCATCAGCAG |
| | CCTGCAGTCTGAAGATTTTGCAGTTTATTACTGTCAGCAGCATAATA |
| | ACTGGCCTCTCACTTTCGGCGGCGGGACCAAGGTGGAAATCAAAC |
| 271 | GAGGTGCAGCTGGTGGAGTCCGGGGGAGGCTTAGTTCAGCCTGGG |
| | GGGTCCCTGAGACTCTCCTGTGCAGCCTCTGGATTCACCTTCAGTAG |
| | CTACTGGATGCACTGGGTCCGCCAAGCTCCAGGGAAGGGGCTGGTG |
| | TGGGTCTCACGTATTAAGAGTGATGGGAGGAGCATAAGTTACGCGG |
| | ACTCCGTGAAGGGCCGATTCACCATCTCCAGAGACAACGCCAAGAA |
| | CACGCTGTATCTGCAAATGAACAGTCTGAGAGCCGAGGACACGGCT |
| | GTGTATTACTGTGCAAGAGATCCCCACGGCACAGCAGCTCCTCCCC |
| | GTGATGCTTTTGATATCTGGGGCCAAGGGACAATGGTCACCGTCTC |
| 272 | CAGTCTGCCCTGACTCAGCCTGCCTCCGTGTCTGGGTCTCCTGGACA |
| | GTCGATCACCATCTCCTGCACTGGAACCAGCGGTGATGTTGGGAGT |
| | TATAACCTTGTCTCCTGGTACCAACAGTACCCAGGCAAAGCCCCCA |
| | AACTCATGATTTATGAGGTCGATAAGCGGCCCTCAGGGGTCTCTAA |
| | TCGCTTCTCTGGCTCCAAGTCTGGCAACACGGCCTCCCTGACAATTT |
| | CTGGGCTCCGGGCTGAGGACGAGGCTCATTATCACTGCTTCTCATA |
| | TGCAGGTAGTTTGACTTTGGTATTCGGTGGAGGGACCAAGTTGACC |
| | GTCCTAG |
| 273 | CAGTCTGTGCTGACTCAGCCACCCTCAGTGTCTGGGACCCCCGGGC |
| | AGAGGGTCACCATCTCTTGTTCTGGAAGCAGCTCCAACATCGGAAG |
| | TAAAACTGCAAACTGGTACCAGAAGCTCCCAGGAACGGCCCCCAA |
| | ACTCCTCATCTATAGTAACAATCAGCGGCCCTCAGGGGTCCCTGAC |
| | CGATTCTCTGGCTCCAAGTCAGGCACCTCAGCCTCCCTGGCTATCAG |
| | TGGGCTCCAGTCTGAGGATGAGGCTGGGTACTACTGCACAGCATGG |
| | GATGACAGCCTGAATGGTCCGGTGTTCGGCGGAGGGACCAAGCTG |
| | ACCGTCCTAG |
| 274 | GAGGTGCAGCTGGTGGAGTCTGGGGGAGGCTTGGTCCAGCCTGGG |
| | GGGTCCCTGAGACTCTCCTGTGGAGCCTCTGGATTCACCGTCAGTG |
| | GCAAGTATATGACCTGGGCCCGCCAGGCTCCTGAGAAGGGACTGG |
| | AGTGGGTCTCAGCTATCTATCGCGGTGGTGGCACATACTACGCAGA |
| | CTCCGTGAAGGGCAGATTCACCATCTCCAGAGACAATTCCAAAAAC |
| | ATGTTATATCTTCAAATGAACAGCCTGAGAGCCGAGGACACGGCTG |
| | TGTATTACTGTGGGGGGTCCGTGATGGTGAGTGCTACTGACTACTG |
| | GGGCCAGGGAACCCTGGTCACCGTCTC |
| 275 | GACATCCAGATGACCCAGTCTCCATCCTCCCTGTCTGCATCTGTAGG |
| | AGACAGAGTCACCATCACTTGCCGGGCAAGTCAGAGCATTAGCAGC |
| | TATTTAAATTGGTATCAGCAGAAACCAGGGAAAGCCCCTAAGCTCC |
| | TGATCTATGCTGCATCCAGTTTGCAAAGTGGGGTCCCATCAAGGTT |
| | CAGTGGCAGTGGATCTGGGACAGATTTCACTCTCACCATCAGCAGT |
| | CTGCAACCTGAAGATTTTGCAACTTACTACTGTCAACAGAGTTACA |
| | GTACCCCGTACACTTTTGGCCAGGGGACCAAGGTGGAGATCAAAC |
| 276 | GAGGTGCAGCTGGTGGAGTCTGGGGGAGGCCTGGTCAAGCCTGGG |
| | GGGTCCCTGAGACTCTCCTGTGCAGCCTCTGGATTCACCTTCAGTAG |
| | CTATAGCATGAACTGGGTCCGCCAGGCTCCAGGGAAGGGGCTGGA |
| | GTGGGTCTCATCCATTAGTAGTAGTAGTAGTTACATATACTACGCA |
| | GACTCAGTGAAGGGCCGATTCACCATCTCCAGAGACAACGCCAAG |
| | AACTCACTGTATCTGCAAATGAACAGCCTGAGAGCCGAGGACACG |
| | GCTGTGTATTACTGTGCGAGAGATCAAATTAGTGGTTACTGGGGCC |
| | AGGGAACCCTGGTCACCGTCTC |
| 277 | GAGGTGCAGCTGGTGGAGTCCGGGGGAGGCTTAGTTCAGCCTGGG |
| | GGGTCCCTGAGACTCTCCTGTGTAGCCTCTGGATTCACCTTCAGTAG |
| | CTACTGGATGCACTGGGTCCGCCAAGCTCCAGGGAAGGGGCTGGTG |
| | TGGGTCTCACGGATTTATCCTGATGGGACTACTACTGCAAACTACG |
| | CGGACTCCGTGAAGGGCCGATTCACCATCTCCAGAGACAACGCCAA |
| | GAACACGGTGTATCTGCAAATGAACAGTCTGAGAGCCGAGGACAC |
| | GGCTGTTTATTACTGTGCAAGAGATCTTCGGGAGTCTGATTACTGG |
| | GGCCAGGGAACCCTGGTCACCGTCTC |
| 278 | CAGTCTGCCCTGACTCAGCCTGCCTCCGTGTCTGGGTCTCCTGGACA |
| | GTCGATCACCATCTCCTGCACTGGAACCAGCAGTGACGTGGGTAAT |
| | TATAATTATGTCTCCTGGCACCAACAACACCCAGGGAAAGCCCCCA |
| | AACTCATGATTTATGATGTCAGTAATCGGCCCTCAGGGGTTTCTAAT |
| | CGCTTCTCTGGCTCCAAGTCTGGCAACACGGCCTCCCTGACCATCTC |
| | TGGGCTCCAGGCTGAGGACGAGGCTGATTATTACTGCAACTCATAT |
| | ACAACCTACAGCACTCACGTCTTCGGAACTGGGACCAAGGTCACCG |
| | TCCTAG |
| 279 | KPAVIPDREALYQDIDEMEEC |
| 280 | KPAVIPDREMLIQSIVEMEEC |
| 281 | PAVIPDRPMLFQSIVEMEEC |
| 282 | PMLF(V,T)QSIV |
| 283 | PMLFQSIV |
| 284 | GAGGTGCAGCTGGTGGAGTCTGGGGGAGGCGTGGCCCAGCCTGGG |
| | ACGTCTCTGAGACTCTCCTGTGAAGCGTCTGGATTCACCTTCCGTGA |
| | CTATGCCATGCGCTGGGTCCGCCAGGCTCCAGGCAAGGGGCTGGAG |
| | TGGGTGGCATTTATATGGAATGATGGAAGTAAGAAATATTACACAG |
| | ACTCCGTGAGGGGCCGATTCACCATCTCCAGAGACAATTCCAAGAA |
| | CATGCTGTATCTGCAAATGGACAGCCTGAGAGCCGAGGACACGGCT |
| | CTTTATTACTGTGCCAGAAAGATGAGTGAAGATGATGCTTTTGATCT |
| | GTGGGGCCAAGGGACAATGGTCACCGTCT |
| 285 | GAGGTGCAGCTGGTGGAGTCCGGGGGAGGCTTAGTTCAGCCGGGG |
| | GGGTCCCTGAGACTCTCCTGTGCAGCCTCTGGATTCACATTCAGTAG |
| | TTACTGGATGGAATGGGTCCGCCAAGCTCCAGGGAAGGGGCTGGTT |
| | TGGGTCTCACATAAAGATATTAGTAGTGATGGGAGTGTTACAAGGT |
| | ACGTGGACTCCGTGAAGGGCCGATTCACCATCTCCAGAGACAACGC |
| | CAAGAACACGCTGTATCTGCAAATGAATAGTCTGAGAGCCGAGGA |
| | CACGGGTGTATATTATTGTGCAAAAGATCTTCACTGGAACGCTCTT |
| | GATGTGTGGGGCCAAGGGACAATGGTCACCGTCTCG |
| 286 | QSALTQPRSVSGSPGQSVTISCTGTSSDVGGYNYVSWYQQHPGKAPKL |
| | MIYDVSKRPSGVPDRFSGSKSGNTASLTISGLQAEDEADYYCCSYAGS |
| | YTLLFGGGTKLTVL |
| 287 | CSYAGSYTLL |
| 288 | CAGTCTGCCCTGACTCAGCCTCGCTCAGTGTCCGGGTCTCCTGGACA |
| | GTCAGTCACCATCTCCTGCACTGGAACCAGCAGTGATGTTGGTGGT |
| | TATAACTATGTCTCCTGGTACCAACAGCACCCAGGCAAAGCCCCCA |
| | AACTCATGATTTATGATGTCAGTAAGCGGCCCTCAGGGGTCCCTGA |
| | TCGCTTCTCTGGCTCCAAGTCTGGCAACACGGCCTCCCTGACCATCT |
| | CTGGGCTCCAGGCTGAGGATGAGGCTGATTATTACTGCTGCTCATA |
| | TGCAGGCAGCTACACTTTGTTATTCGGCGGAGGGACCAAGCTGACC |
| | GTCCTAG |
| 289 | EVQLVESGGGLVQPGGSLRLSCAASGFTFSSYWMYWVRQAPGKGLV |
| | WVSRINSDGSTTNYADSVKGRFTISRDNAKNTLYLQMNSLRAEDTAV |
| | YFCARYAHLGIGWYFDLWGRGTLVTVSS |
| 290 | INSDGSTT |
| 291 | ARYAHLGIGWYFDL |
| 292 | GAGGTGCAGCTGGTGGAGTCCGGGGGAGGCTTAGTTCAGCCTGGG |
| | GGGTCCCTGAGACTCTCCTGTGCAGCCTCTGGATTCACCTTCAGTAG |
| | CTACTGGATGTACTGGGTCCGCCAAGCTCCAGGGAAGGGGCTGGTG |
| | TGGGTCTCACGTATTAATAGTGATGGGAGTACCACGAACTACGCGG |
| | ACTCCGTGAAGGGCCGATTCACCATCTCCAGAGACAACGCCAAGAA |
| | CACGCTGTATCTGCAAATGAACAGTCTGAGAGCCGAGGACACGGCT |
| | GTGTATTTCTGTGCAAGATATGCCCACCTGGGGATAGGTTGGTACTT |
| | CGATCTCTGGGGCCGTGGCACCCTGGTCACCGTCTCCTCAG |
| 293 | DIQMTQSPSTLSASAGDRVTITCRASQSVSKYLAWYQQKPGKAPRLLI |
| | YKASSLQSGVPSRFSGSGSGTEFTLTISSLQPDDFATYYCQHYNTYPSW |
| | TFGQGTKVEIK |
| 294 | QSVSKY |
| 295 | KAS |
| 296 | QHYNTYPSWT |
| 297 | GACATCCAGATGACCCAGTCTCCTTCCACCCTGTCTGCATCTGCAGG |
| | AGACAGAGTCACCATCACTTGCCGGGCCAGTCAGAGTGTTAGTAAG |
| | TACTTGGCCTGGTATCAGCAGAAACCAGGGAAAGCCCCTAGGCTCC |
| | TGATCTATAAGGCATCTAGTTTACAAAGTGGGGTCCCATCAAGGTT |
| | CAGTGGCAGTGGATCTGGGACAGAATTCACTCTCACCATCAGCAGC |
| | CTGCAGCCTGATGATTTTGCTACTTATTACTGCCAACACTATAATAC |
| | TTATCCTTCGTGGACGTTCGGCCAAGGGACCAAGGTGGAAATCAAA |
| 298 | EVQLVESGGGVAQPGTSLRLSCEASGFTFRDYAMRWVRQAPGKGLE |
| | WVAFIWNDGSKKYYTDSVRGRFTISRDNSKNMLYLQMDSLRAEDTAL |
| | YYCARKMSEDDAFDLWGQGTMVTV |
| 299 | GFTFRDYA |
| 300 | IWNDGSKK |
| 301 | ARKMSEDDAFDL |
| 302 | DIQMTQSPSSLSASVGDRVTITCRASQGIRNDLGWYQQKPGKAPKLLIY |
| | GASSLQSGVPSRFSGSGSGTDFTLTISSLQPEDIATYYCLQEYTYPLTFG |
| | GGTKVEIK |
| 303 | QGIRND |
| 304 | LQEYTYPLT |
| 305 | GACATCCAGATGACCCAGTCTCCATCCTCCCTGTCTGCATCCGTAGG |
| | AGACAGAGTCACCATCACTTGCCGGGCAAGTCAGGGCATTAGAAAT |
| | GATTTAGGCTGGTATCAGCAGAAACCAGGGAAAGCCCCTAAGCTCC |
| | TGATCTATGGTGCATCCAGTTTACAAAGTGGGGTCCCATCAAGATT |
| | CAGCGGCAGTGGATCTGGCACAGATTTCACTCTCACCATCAGCAGC |
| | CTGCAGCCTGAAGATATTGCAACCTATTACTGTCTACAAGAATACA |
| | CTTACCCGCTCACTTTCGGCGGAGGGACCAAGGTGGAAATCAAA |
| 306 | EVQLVESGGGLVQPGGSLRLSCAASGFTFSDYYMDWVRQAPGKGLE |
| | WVGRTRNKANSYTTEYAASVKGRFTISRDDSKNSLYLQMNSLKTEDT |
| | AVYYCARFLLVADAFDIWGQGTMVTV |
| 307 | GFTFSDYY |
| 308 | TRNKANSYTT |
| 309 | ARFLLVADAFDI |
| 310 | GAGGTGCAGCTGGTGGAGTCTGGGGGAGGCTTGGTCCAGCCTGGA |
| | GGGTCCCTGAGACTCTCCTGTGCAGCCTCTGGATTCACCTTCAGTGA |
| | CTACTACATGGACTGGGTCCGCCAGGCTCCAGGGAAGGGGCTGGA |
| | GTGGGTTGGCCGCACTAGAAACAAAGCTAACAGTTACACCACAGA |
| | ATACGCCGCGTCTGTGAAAGGCAGATTCACCATCTCAAGAGATGAT |
| | TCAAAGAACTCACTGTATCTGCAAATGAACAGCCTGAAAACCGAGG |
| | ACACGGCCGTGTATTACTGTGCTAGATTTCTGCTGGTTGCGGATGCT |
| | TTTGATATCTGGGGCCAAGGGACAATGGTCACCGTCT |
| 311 | AGKPAVIPDREALYQDIDEMEECLDEAGKPAVIPDREALYQDIDEMEE |
| | CLDEAGKPAVIPDREALYQDIDEMEECLD |
| 312 | IYPLEDMAEPKVERIDAQEDC |

### EMBODIMENTS

1. An isolated antibody or antigen binding fragment thereof comprising:
a heavy chain variable domain comprising:
a CDRH1 region selected from the group consisting of SEQ ID NOs: 2, 14, 22, 30, 42, 50, 62, 78, 86, 93, 101, 123, 126, 139, 149, 152, 164, 176, 186, and 205;
a CDRH2 region selected from the group consisting of SEQ ID NOs: 74, 15, 23, 31, 35, 43, 51, 63, 70, 75, 79, 87, 94, 102, 111, 114, 127, 136, 140, 153, 165, 168, 177, 183, 187, 193, 206, and 212;
a CDRH3 region selected from the group consisting of SEQ ID NOs: 4, 16, 14, 32, 36, 44, 52, 64, 71, 76, 80, 88, 95, 103, 112, 115, 124, 128, 137, 141, 150, 154, 166, 169, 178, 184, 188, 194, 207, 210, and 213; and
a light chain variable domain comprising:
a CDRL1 region selected from the group consisting of SEQ ID NOs: 6, 10, 18, 26, 37, 46, 54, 58, 66, 38, 82, 97, 105, 46, 119, 129, 132, 143, 146, 156, 160, 171, 180, 190, 197, 201, and 215;
a CDRL2 region selected from the group consisting of SEQ ID NOs: 7, 11, 27, 28, 47, 55, 59, 67, 39, 83, 98, 106, 120, 133, 157, 161, 198, 202, and 208; and
a CDRL3 region selected from the group consisting of SEQ ID NOs: 8, 12, 20, 28, 39, 48, 56, 60, 68, 73, 84, 99, 107, 109, 117, 121, 130, 134, 144, 147, 158, 162, 172, 174, 181, 191, 199, 203, and 216.

2. The antibody or antigen binding fragment thereof of embodiment 1, wherein
(a) the heavy chain variable region is encoded by SEQ ID NO: 1 and the light chain variable region is encoded by SEQ ID NO: 5;
or
(b) the heavy chain variable region is encoded by SEQ ID NO: 13 and the light chain variable region is encoded by SEQ ID NO: 9;
or
(c) the heavy chain variable region is encoded by SEQ ID NO: 21 and the light chain variable region is encoded by SEQ ID NO: 17;
or
(d) the heavy chain variable region is encoded by SEQ ID NO: 29 and the light chain variable region is encoded by SEQ ID NO: 25;
or
(e) the heavy chain variable region is encoded by SEQ ID NO: 33 and the light chain variable region is encoded by SEQ ID NO: 37;
or
(f) the heavy chain variable region is encoded by SEQ ID NO: 41 and the light chain variable region is encoded by SEQ ID NO: 45;
or
(g) the heavy chain variable region is encoded by SEQ ID NO: 49 and the light chain variable region is encoded by SEQ ID NO: 53;
or
(h) the heavy chain variable region is encoded by SEQ ID NO: 61 and the light chain variable region is encoded by SEQ ID NO: 57;
or
(i) the heavy chain variable region is encoded by SEQ ID NO: 69 and the light chain variable region is encoded by SEQ ID NO: 65;
or
(j) the heavy chain variable region is encoded by SEQ ID NO: 74 and the light chain variable region is encoded by SEQ ID NO: 72;
or
(k) the heavy chain variable region is encoded by SEQ ID NO: 77 and the light chain variable region is encoded by SEQ ID NO: 81;
or
(l) the heavy chain variable region is encoded by SEQ ID NO: 85 and the light chain variable region is encoded by SEQ ID NO: 89;
or
(m) the heavy chain variable region is encoded by SEQ ID NO: 92 and the light chain variable region is encoded by SEQ ID NO: 96;
or
(n) the heavy chain variable region is encoded by SEQ ID NO: 100 and the light chain variable region is encoded by SEQ ID NO: 104;
or
(o) the heavy chain variable region is encoded by SEQ ID NO: 110 and the light chain variable region is encoded by SEQ ID NO: 108;
or
(p) the heavy chain variable region is encoded by SEQ ID NO: 113 and the light chain variable region is encoded by SEQ ID NO: 116;
or
(q) the heavy chain variable region is encoded by SEQ ID NO: 122 and the light chain variable region is encoded by SEQ ID NO: 118;
or
(r) the heavy chain variable region is encoded by SEQ ID NO: 125 and the light chain variable region is encoded by SEQ ID NO: 313;
or
(s) the heavy chain variable region is encoded by SEQ ID NO: 135 and the light chain variable region is encoded by SEQ ID NO: 131;
or
(t) the heavy chain variable region is encoded by SEQ ID NO: 138 and the light chain variable region is encoded by SEQ ID NO: 142;
or
(u) the heavy chain variable region is encoded by SEQ ID NO: 148 and the light chain variable region is encoded by SEQ ID NO: 145;
or
(v) the heavy chain variable region is encoded by SEQ ID NO: 151 and the light chain variable region is encoded by SEQ ID NO: 155;
or
(w) the heavy chain variable region is encoded by SEQ ID NO: 163 and the light chain variable region is encoded by SEQ ID NO: 159;
or
(x) the heavy chain variable region is encoded by SEQ ID NO: 167 and the light chain variable region is encoded by SEQ ID NO: 170;
or
(y) the heavy chain variable region is encoded by SEQ ID NO: 175 the light chain variable region is encoded by SEQ ID NO: 173;
or
(z) the heavy chain variable region is encoded by SEQ ID NO: 182 the light chain variable region is encoded by SEQ ID NO: 179;
or
(aa) the heavy chain variable region is encoded by SEQ ID NO: 185 the light chain variable region is encoded by SEQ ID NO: 189;
or
(bb) the heavy chain variable region is encoded by SEQ ID NO: 192 the light chain variable region is encoded by SEQ ID NO: 196;
or
(cc) the heavy chain variable region is encoded by SEQ ID NO: 204 the light chain variable region is encoded by SEQ ID NO: 200;
or
(dd) the heavy chain variable region is encoded by SEQ ID NO: 209 the light chain variable region is encoded by SEQ ID NO: 159;
or
(ee) the heavy chain variable region is encoded by SEQ ID NO: 211 the light chain variable region is encoded by SEQ ID NO: 214.

3. The antibody or antigen binding fragment thereof of embodiment 1 or embodiment 2, wherein
(a) a heavy chain variable domain comprising a CDRH1 region consisting of SEQ ID NO: 42, a CDRH2 region consisting of SEQ ID NO: 74, and a CDRH3 region consisting of SEQ ID NO: 4; and a light chain variable domain comprising a CDRL1 region consisting of SEQ ID NO: 6, a CDRL2 region consisting of SEQ ID NO: 7, and a CDRL3 region consisting of SEQ ID NO: 8;
or
(b) a heavy chain variable domain comprising a CDRH1 region consisting of SEQ ID NO: 14, a CDRH2 region consisting of SEQ ID NO: 15, and a CDRH3 region consisting of SEQ ID NO: 16; and a light chain variable domain comprising a CDRL1 region consisting of SEQ ID NO: 10, a CDRL2 region consisting of SEQ ID NO: 11, and a CDRL3 region consisting of SEQ ID NO: 12;
or
(c) a heavy chain variable domain comprising a CDRH1 region consisting of SEQ ID NO: 22, a CDRH2 region consisting of SEQ ID NO: 23, and a CDRH3 region consisting of SEQ ID NO: 14; and a light chain variable domain comprising a CDRL1 region consisting of SEQ ID NO: 18, a CDRL2 region consisting of SEQ ID NO: 11, and a CDRL3 region consisting of SEQ ID NO: 20;
or
(d) a heavy chain variable domain comprising a CDRH1 region consisting of SEQ ID NO: 30, a CDRH2 region consisting of SEQ ID NO: 31, and a CDRH3 region consisting of SEQ ID NO: 32; and a light chain variable domain comprising a CDRL1 region consisting of SEQ ID NO: 26, a CDRL2 region consisting of SEQ ID NO: 27, and a CDRL3 region consisting of SEQ ID NO: 28;
or
(e) a heavy chain variable domain comprising a CDRH1 region consisting of SEQ ID NO: 78, a CDRH2 region consisting of SEQ ID NO: 35, and a CDRH3 region consisting of SEQ ID NO: 36; and a light chain variable domain comprising a CDRL1 region consisting of SEQ ID NO: 37, a CDRL2 region consisting of SEQ ID NO: 38, and a CDRL3 region consisting of SEQ ID NO: 39;
or
(f) a heavy chain variable domain comprising a CDRH1 region consisting of SEQ ID NO: 42, a CDRH2 region consisting of SEQ ID NO: 43, and a CDRH3 region consisting of SEQ ID NO: 44; and a light chain variable domain comprising a CDRL1 region consisting of SEQ ID NO: 46, a CDRL2 region consisting of SEQ ID NO: 47, and a CDRL3 region consisting of SEQ ID NO: 48;
or
(g) a heavy chain variable domain comprising a CDRH1 region consisting of SEQ ID NO: 50, a CDRH2 region consisting of SEQ ID NO: 51, and a CDRH3 region consisting of SEQ ID NO: 52; and a light chain variable domain comprising a CDRL1 region consisting of SEQ ID NO: 54, a CDRL2 region consisting of SEQ ID NO: 55, and a CDRL3 region consisting of SEQ ID NO: 56;
or
(h) a heavy chain variable domain comprising a CDRH1 region consisting of SEQ ID NO: 62, a CDRH2 region consisting of SEQ ID NO: 63, and a CDRH3 region consisting of SEQ ID NO: 64; and a light chain variable domain comprising a CDRL1 region consisting of SEQ ID NO: 58, a CDRL2 region consisting of SEQ ID NO: 59, and a CDRL3 region consisting of SEQ ID NO: 60;
or
(i) a heavy chain variable domain comprising a CDRH1 region consisting of SEQ ID NO: 42, a CDRH2 region consisting of SEQ ID NO: 70, and a CDRH3 region consisting of SEQ ID NO: 71; and a light chain variable domain comprising a CDRL1 region consisting of SEQ ID NO: 66, a CDRL2 region consisting of SEQ ID NO: 67, and a CDRL3 region consisting of SEQ ID NO: 68;
or
(j) a heavy chain variable domain comprising a CDRH1 region consisting of SEQ ID NO: 42, a CDRH2 region consisting of SEQ ID NO: 75, and a CDRH3 region consisting of SEQ ID NO: 76; and a light chain variable domain comprising a CDRL1 region consisting of SEQ ID NO: 38, a CDRL2 region consisting of SEQ ID NO: 39, and a CDRL3 region consisting of SEQ ID NO: 73;
or
(k) a heavy chain variable domain comprising a CDRH1 region consisting of SEQ ID NO: 78, a CDRH2 region consisting of SEQ ID NO: 79, and a CDRH3 region consisting of SEQ ID NO: 80; and a light chain variable domain comprising a CDRL1 region consisting of SEQ ID NO: 82, a CDRL2 region consisting of SEQ ID NO: 83, and a CDRL3 region consisting of SEQ ID NO: 84;
or
(l) a heavy chain variable domain comprising a CDRH1 region consisting of SEQ ID NO: 86, a CDRH2 region consisting of SEQ ID NO: 87, and a CDRH3 region consisting of SEQ ID NO: 88; and a light chain variable domain comprising a CDRL1 region consisting of SEQ ID NO: 82, a CDRL2 region consisting of SEQ ID NO: 83, and a CDRL3 region consisting of SEQ ID NO: 84;
or
(m) a heavy chain variable domain comprising a CDRH1 region consisting of SEQ ID NO: 93, a CDRH2 region consisting of SEQ ID NO: 94, and a CDRH3 region consisting of SEQ ID NO: 95; and a light chain variable domain comprising a CDRL1 region consisting of SEQ ID NO: 97, a CDRL2 region consisting of SEQ ID NO: 98, and a CDRL3 region consisting of SEQ ID NO: 99;
or
(n) a heavy chain variable domain comprising a CDRH1 region consisting of SEQ ID NO: 101, a CDRH2 region consisting of SEQ ID NO: 102, and a CDRH3 region consisting of SEQ ID NO: 103; and a light chain variable domain comprising a CDRL1 region consisting of SEQ ID NO: 105, a CDRL2 region consisting of SEQ ID NO: 106, and a CDRL3 region consisting of SEQ ID NO: 107;
or
(o) a heavy chain variable domain comprising a CDRH1 region consisting of SEQ ID NO: 42, a CDRH2 region consisting of SEQ ID NO: 111, and a CDRH3 region consisting of SEQ ID NO: 112; and a light chain variable domain comprising a CDRL1 region consisting of SEQ ID NO: 46, a CDRL2 region consisting of SEQ ID NO: 67, and a CDRL3 region consisting of SEQ ID NO: 109;
or
(p) a heavy chain variable domain comprising a CDRH1 region consisting of SEQ ID NO: 2, a CDRH2 region consisting of SEQ ID NO: 114, and a CDRH3 region consisting of SEQ ID NO: 115; and a light chain variable domain comprising a CDRL1 region consisting of SEQ ID NO: 46, a CDRL2 region consisting of SEQ ID NO: 47, and a CDRL3 region consisting of SEQ ID NO: 117;
or
(q) a heavy chain variable domain comprising a CDRH1 region consisting of SEQ ID NO: 123, a CDRH2 region consisting of SEQ ID NO: 75, and a CDRH3 region consisting of SEQ ID NO: 124; and a light chain variable domain comprising a CDRL1 region consisting of SEQ ID NO: 119, a CDRL2 region consisting of SEQ ID NO: 120, and a CDRL3 region consisting of SEQ ID NO: 121;
or
(r) a heavy chain variable domain comprising a CDRH1 region consisting of SEQ ID NO: 126, a CDRH2 region consisting of SEQ ID NO: 127, and a CDRH3 region consisting of SEQ ID NO: 128; and a light chain variable domain comprising a CDRL1 region consisting of SEQ ID NO: 129, a CDRL2 region consisting of SEQ ID NO: 39, and a CDRL3 region consisting of SEQ ID NO: 130;
or
(s) a heavy chain variable domain comprising a CDRH1 region consisting of SEQ ID NO: 78, a CDRH2 region consisting of SEQ ID NO: 136, and a CDRH3 region consisting of SEQ ID NO: 137; and a light chain variable domain comprising a CDRL1 region consisting of SEQ ID NO: 132, a CDRL2 region consisting of SEQ ID NO: 133, and a CDRL3 region consisting of SEQ ID NO: 134;
or
(t) a heavy chain variable domain comprising a CDRH1 region consisting of SEQ ID NO: 139, a CDRH2 region consisting of SEQ ID NO: 140, and a CDRH3 region consisting of SEQ ID NO: 141; and a light chain variable domain comprising a CDRL1 region consisting of SEQ ID NO: 143, a CDRL2 region consisting of SEQ ID NO: 67, and a CDRL3 region consisting of SEQ ID NO: 144;
or
(u) a heavy chain variable domain comprising a CDRH1 region consisting of SEQ ID NO: 149, a CDRH2 region consisting of SEQ ID NO: 75, and a CDRH3 region consisting of SEQ ID NO: 150; and a light chain variable domain comprising a CDRL1 region consisting of SEQ ID NO: 146, a CDRL2 region consisting of SEQ ID NO: 11, and a CDRL3 region consisting of SEQ ID NO: 147;
or
(v) a heavy chain variable domain comprising a CDRH1 region consisting of SEQ ID NO: 152, a CDRH2 region consisting of SEQ ID NO: 153, and a CDRH3 region consisting of SEQ ID NO: 154; and a light chain variable domain comprising a CDRL1 region consisting of SEQ ID NO: 156, a CDRL2 region consisting of SEQ ID NO: 157, and a CDRL3 region consisting of SEQ ID NO: 158;
or
(w) a heavy chain variable domain comprising a CDRH1 region consisting of SEQ ID NO: 164, a CDRH2 region consisting of SEQ ID NO: 165, and a CDRH3 region consisting of SEQ ID NO: 166; and a light chain variable domain comprising a CDRL1 region consisting of SEQ ID NO: 160, a CDRL2 region consisting of SEQ ID NO: 161, and a CDRL3 region consisting of SEQ ID NO: 162;
or
(x) a heavy chain variable domain comprising a CDRH1 region consisting of SEQ ID NO: 164, a CDRH2 region consisting of SEQ ID NO: 168, and a CDRH3 region consisting of SEQ ID NO: 169; and a light chain variable domain comprising a CDRL1 region consisting of SEQ ID NO: 171, a CDRL2 region consisting of SEQ ID NO: 7, and a CDRL3 region consisting of SEQ ID NO: 172;
or
(y) a heavy chain variable domain comprising a CDRH1 region consisting of SEQ ID NO: 176, a CDRH2 region consisting of SEQ ID NO: 177, and a CDRH3 region consisting of SEQ ID NO: 178; and a light chain variable domain comprising a CDRL1 region consisting of SEQ ID NO: 38, a CDRL2 region consisting of SEQ ID NO: 39, and a CDRL3 region consisting of SEQ ID NO: 174;
or
(z) a heavy chain variable domain comprising a CDRH1 region consisting of SEQ ID NO: 42, a CDRH2 region consisting of SEQ ID NO: 183, and a CDRH3 region consisting of SEQ ID NO: 184; and a light chain variable domain comprising a CDRL1 region consisting of SEQ ID NO: 180, a CDRL2 region consisting of SEQ ID NO: 39, and a CDRL3 region consisting of SEQ ID NO: 181;
or
(aa) a heavy chain variable domain comprising a CDRH1 region consisting of SEQ ID NO: 186, a CDRH2 region consisting of SEQ ID NO: 187, and a CDRH3 region consisting of SEQ ID NO: 188; and a light chain variable domain comprising a CDRL1 region consisting of SEQ ID NO: 190, a CDRL2 region consisting of SEQ ID NO: 106, and a CDRL3 region consisting of SEQ ID NO: 191;
or
(bb) a heavy chain variable domain comprising a CDRH1 region consisting of SEQ ID NO: 42, a CDRH2 region consisting of SEQ ID NO: 193, and a CDRH3 region consisting of SEQ ID NO: 194; and a light chain variable domain comprising a CDRL1 region consisting of SEQ ID NO: 197, a CDRL2 region consisting of SEQ ID NO: 198, and a CDRL3 region consisting of SEQ ID NO: 199;
or
(cc) a heavy chain variable domain comprising a CDRH1 region consisting of SEQ ID NO: 205, a CDRH2 region consisting of SEQ ID NO: 206, and a CDRH3 region consisting of SEQ ID NO: 207; and a light chain variable domain comprising a CDRL1 region consisting of SEQ ID NO: 201, a CDRL2 region consisting of SEQ ID NO: 202, and a CDRL3 region consisting of SEQ ID NO: 203;
or
(dd) a heavy chain variable domain comprising a CDRH1 region consisting of SEQ ID NO: 164, a CDRH2 region consisting of SEQ ID NO: 165, and a CDRH3 region consisting of SEQ ID NO: 210; and a light chain variable domain comprising a CDRL1 region consisting of SEQ ID NO: 160, a CDRL2 region consisting of SEQ ID NO: 208, and a CDRL3 region consisting of SEQ ID NO: 162;
or
(ee) a heavy chain variable domain comprising a CDRH1 region consisting of SEQ ID NO: 42, a CDRH2 region consisting of SEQ ID NO: 212, and a CDRH3 region consisting of SEQ ID NO: 213; and a light chain variable domain comprising a CDRL1 region consisting of SEQ ID NO: 215, a CDRL2 region consisting of SEQ ID NO: 47, and a CDRL3 region consisting of SEQ ID NO: 216.

4. The antibody or antigen binding fragment thereof of any of embodiment 1-3, wherein the antibody is a chimeric antibody and the heavy chain constant domain is from rabbit, mouse, rat, or nonhuman primate.

5. The antibody or antigen binding fragment thereof of any of embodiment 1-4, wherein the light chain constant domain is a kappa light chain constant domain or a lambda light chain constant domain.

6. The antibody or antigen binding fragment thereof of any of embodiment 1-5, wherein the antibody is linked to a detectable label.

7. A peptide comprising SEQ ID NO: 281 or a peptide comprising a sequence with 95% similarity to SEQ ID NO: 281.

8. The peptide of embodiment 7, further comprising a detectable label.

9. The peptide of embodiment 7 or 8, wherein the peptide is linked to a solid support.

10. A method of diagnosing Kawasaki Disease in a subject, comprising the steps of:
i) obtaining a sample from a subject suspected of having Kawasaki Disease;
ii) contacting the sample with the antibody or antigen binding fragment thereof of any of embodiment 1-3; and
iii) detecting the binding of the antibody to a component of the sample, whereby binding of the antibody to the component of the sample indicates the presence of Kawasaki Disease and confirms the diagnosis of Kawasaki disease in the subject.

11. The method of embodiment 10, further comprising
iv) treating the subject diagnosed with Kawasaki disease with intravenous immunoglobulin (IV Ig).

12. The method of embodiment 10 or embodiment 11, wherein the sample is a blood sample or a serum sample.

13. The method of any of embodiment 10-12, wherein detecting the binding of the antibody or antigen binding fragment thereof in the sample is carried out using ELISA, Western blot, immunostaining, immunoprecipitation, flow cytometry, sensor chips, or magnetic beads.

14. The method of any of embodiment 10-13, wherein the antibody or antigen binding fragment thereof is linked to a solid support.

15. The method of embodiment 14, wherein detecting the binding of the antibody to the component of the sample comprises contacting the sample with an antibody or antigen fragment thereof of any of embodiment 1-3.

16. A method of detecting intracytoplasmic inclusion bodies in a subject, comprising the steps of:
i) obtaining a sample from a subject suspected of having Kawasaki Disease;
ii) contacting the sample with the antibody or antigen binding fragment thereof of any of embodiment 1-3; and
iii) detecting the binding of the antibody or antigen binding fragment thereof in the sample, whereby binding of the antibody indicates the presence of intracytoplasmic inclusion bodies as compared to a negative control.

17. The method of embodiment 16, further comprising
iv) treating the subject having detected antibodies associated with Kawasaki disease with intravenous immunoglobulin (IV Ig).

18. A method of detecting antibodies associated with Kawasaki disease in a subject comprising the steps of:
i) obtaining a sample comprising antibodies from a subject suspected of having Kawasaki disease;
ii) contacting the sample with the peptide of any of embodiment 7-9; and
iii) detecting the binding of antibodies to the peptide to form a peptide-antibody complex, wherein the presence of a peptide-antibody complex confirms the presence of antibodies associated with Kawasaki disease in the subject.

19. The method of embodiment 18, further comprising

iv) treating the subject having detected antibodies associated with Kawasaki disease with intravenous immunoglobulin (IV Ig).

20. The method of embodiment 18 or 19, wherein the peptide is linked to a solid support.

21. The method of any of embodiment 18-20, wherein detecting comprises contacting the peptide-antibody complex with a secondary antibody wherein the secondary antibody is optionally linked to a detectable label.

22. The method of embodiment 21, wherein the secondary antibody is an anti-human Fc antibody.

23. A kit comprising:
i) the antibody or antigen binding fragment thereof of any of embodiment 1-3;
ii) a detection reagent.

24. The kit of embodiment 23, further comprising:
iii) a solid support.

25. The kit of embodiment 24, wherein the antibody or antigen binding fragment thereof is linked to the solid support.

26. The kit of embodiment 24 or 25, wherein the solid support comprises a lateral flow device.

27. The kit of any of embodiment 23-26, wherein the detection reagent comprises an antibody or antigen binding fragment thereof of any of embodiment 1-3.

28. A kit comprising:
i) a peptide of any of embodiment 7-9;
ii) a detection reagent.

29. The kit of embodiment 28, further comprising:
iii) a solid support, wherein the peptide is linked to the solid support.

30. The kit of embodiment 29, wherein the detection reagent comprises a secondary antibody optionally linked to a detectable label.

### REFERENCES

1. McCrindle, B. W. et al. Diagnosis, Treatment, and Long-Term Management of Kawasaki Disease: A Scientific Statement for Health Professionals From the American Heart Association. Circulation 135, e927-e999 (2017).
2. Orenstein, J. M. et al. Three linked vasculopathic processes characterize Kawasaki disease: a light and transmission electron microscopic study. PLoS One 7, e38998 (2012).
3. Amano, S. et al. General pathology of Kawasaki disease. On the morphological alterations corresponding to the clinical manifestations. Acta Pathol. Jpn. 30, 681-694 (1980).
4. Whittaker, E. et al. Clinical Characteristics of 58 Children With a Pediatric Inflammatory Multisystem Syndrome Temporally Associated With SARS-CoV-2. JAMA 2020 Jun. 9, (2020).
5. Ghosh, P. et al. An Artificial Intelligence-guided signature reveals the shared host immune response in MIS-C and Kawasaki disease. Nat. Commun. 13, 2687 (2022).
6. Wang, Z. et al. Single-cell RNA sequencing of peripheral blood mononuclear cells from acute Kawasaki disease patients. Nature Communications vol. 12 (2021).
7. Rowley, A. H. et al. Cytoplasmic inclusion bodies are detected by synthetic antibody in ciliated bronchial epithelium during acute Kawasaki disease. J. Infect. Dis. 192, 1757-1766 (2005).
8. Rowley, A. H. et al. RNA-containing cytoplasmic inclusion bodies in ciliated bronchial epithelium months to years after acute Kawasaki disease. PLoS One 3, e1582 (2008).
9. Rowley, A. H. et al. Detection of antigen in bronchial epithelium and macrophages in acute Kawasaki disease by use of synthetic antibody. J. Infect. Dis. 190, 856-865 (2004).
10. Rowley, A. H. et al. Ultrastructural, immunofluorescence, and RNA evidence support the hypothesis of a ‘new’ virus associated with Kawasaki disease. J. Infect. Dis. 203, 1021-1030 (2011).
11. Shulman, S., Geevarghese, B., Kim, K. Y. & Rowley, A. The Impact of Social Distancing for COVID-19 Upon Diagnosis of Kawasaki Disease. J Pediatric Infect Dis Soc 2021 Mar. 24, (2021).
12. Kang, J. M. et al. Reduction in Kawasaki Disease After Nonpharmaceutical Interventions in the COVID-19 Era: A Nationwide Observational Study in Korea. Circulation 2021 Jun. 8, (2021).
13. Bailey, L. C. et al. Assessment of 135794 Pediatric Patients Tested for Severe Acute Respiratory Syndrome Coronavirus 2 Across the United States. JAMA Pediatr. 175, 176-184 (2021).
14. Koskela, U. et al. Multi-inflammatory syndrome and Kawasaki disease in children during the COVID-19 pandemic: A nationwide register-based study and time series analysis. Acta Paediatr. 110, 3063-3068 (2021).
15. Ae, R., Shibata, Y., Kosami, K., Nakamura, Y. & Hamada, H. Kawasaki Disease and Pediatric Infectious Diseases During the Coronavirus Disease 2019 Pandemic. J. Pediatr. 239, 50-58.e2 (2021).
16. Rowley, A. H., Shulman, S. T., Spike, B. T., Mask, C. A. & Baker, S. C. Oligoclonal IgA response in the vascular wall in acute Kawasaki disease. J. Immunol. 166, 1334-1343 (2001).
17. Rowley, A. H. et al. A Protein Epitope Targeted by the Antibody Response to Kawasaki Disease. J. Infect. Dis. 222, 158-168 (2020).
18. Rowley, A. H. et al. The transcriptional profile of coronary arteritis in Kawasaki disease. BMC Genomics 16, 1076 (2015).
19. Kreer, C., Gruell, H., Mora, T., Walczak, A. M. & Klein, F. Exploiting B Cell Receptor Analyses to Inform on HIV-1 Vaccination Strategies. Vaccines (Basel) 8, (2020).
20. Zhang, Q. et al. Potent and protective IGHV3-53/3-66 public antibodies and their shared escape mutant on the spike of SARS-CoV-2. Nature Communications vol. 12 (2021).
21. Teng, G. & Papavasiliou, F. N. Immunoglobulin somatic hypermutation. Annu. Rev. Genet. 41, 107-120 (2007).
22. Forsstrom, B. et al. Proteome-wide epitope mapping of antibodies using ultra-dense peptide arrays. Mol. Cell. Proteomics 13, 1585-1597 (2014).
23. Xu, M. et al. Plasmablasts Generated during Repeated Dengue Infection Are Virus Glycoprotein-Specific and Bind to Multiple Virus Serotypes. The Journal of Immunology vol. 189 5877-5885 (2012).
24. Nair, N. et al. High-dimensional immune profiling of total and rotavirus VP6-specific intestinal and circulating B cells by mass cytometry. Mucosal Immunology vol. 9 68-82 (2016).
25. Krammer, F. The human antibody response to influenza A virus infection and vaccination. Nat. Rev. Immunol. 19, 383-397 (2019).
26. Weitkamp, J.-H. et al. Infant and adult human B cell responses to rotavirus share common immunodominant variable gene repertoires. J. Immunol. 171, 4680-4688 (2003).
27. Shulman, S. T. & Rowley, A. H. Kawasaki disease: insights into pathogenesis and approaches to treatment. Nat. Rev. Rheumatol. 11, 475-482 (2015).
28. Vella, L. A. & Rowley, A. H. Current Insights Into the Pathophysiology of Multisystem Inflammatory Syndrome in Children. Curr. Pediatr. Rep. 9, 83-92 (2021).
29. Feldstein, L. R. et al. Multisystem Inflammatory Syndrome in U.S. Children and Adolescents. N. Engl. J. Med. 2020 Jul. 1, (2020).
30. Binstadt, B. A. et al. Coronary artery dilation among patients presenting with systemic-onset juvenile idiopathic arthritis. Pediatrics 116, e89-93 (2005).
31. Mayordomo-Colunga, J. et al. SARS-CoV-2 Spike Protein in Intestinal Cells of a Patient with Coronavirus Disease 2019 Multisystem Inflammatory Syndrome. J. Pediatr. 243, 214-218.e5 (2022).
32. Duarte-Neto, A. N. et al. An autopsy study of the spectrum of severe COVID-19 in children: From SARS to different phenotypes of MIS-C. EClinicalMedicine 35, 100850 (2021).
33. Fox, S. E., Lameira, F. S., Rinker, E. B. & Vander Heide, R. S. Cardiac Endotheliitis and Multisystem Inflammatory Syndrome After COVID-19. Ann. Intern. Med. 173, 1025-1027 (2020).
34. Smith, K. et al. Rapid generation of fully human monoclonal antibodies specific to a vaccinating antigen. Nat. Protoc. 4, 372-384 (2009).

We claim:

1. An isolated antibody or antigen binding fragment thereof comprising:
a heavy chain variable domain comprising:
a CDRH1 region selected from the group consisting of SEQ ID NOs: 2, 14, 22, 30, 42, 50, 62, 78, 86, 93, 101, 123, 126, 139, 149, 152, 164, 176, 186, and 205;
a CDRH2 region selected from the group consisting of SEQ ID NOs: 74, 15, 23, 31, 35, 43, 51, 63, 70, 75, 79, 87, 94, 102, 111, 114, 127, 136, 140, 153, 165, 168, 177, 183, 187, 193, 206, and 212;
a CDRH3 region selected from the group consisting of SEQ ID NOs: 4, 16, 14, 32, 36, 44, 52, 64, 71, 76, 80, 88, 95, 103, 112, 115, 124, 128, 137, 141, 150, 154, 166, 169, 178, 184, 188, 194, 207, 210, and 213; and
a light chain variable domain comprising:
a CDRL1 region selected from the group consisting of SEQ ID NOs: 6, 10, 18, 26, 37, 46, 54, 58, 66, 38, 82, 97, 105, 46, 119, 129, 132, 143, 146, 156, 160, 171, 180, 190, 197, 201, and 215;
a CDRL2 region selected from the group consisting of SEQ ID NOs: 7, 11, 27, 28, 47, 55, 59, 67, 39, 83, 98, 106, 120, 133, 157, 161, 198, 202, and 208; and
a CDRL3 region selected from the group consisting of SEQ ID NOs: 8, 12, 20, 28, 39, 48, 56, 60, 68, 73, 84, 99, 107, 109, 117, 121, 130, 134, 144, 147, 158, 162, 172, 174, 181, 191, 199, 203, and 216.

2. The antibody or antigen binding fragment thereof of claim 1, wherein
(a) the heavy chain variable region is encoded by SEQ ID NO: 1 and the light chain variable region is encoded by SEQ ID NO: 5;
or
(b) the heavy chain variable region is encoded by SEQ ID NO: 13 and the light chain variable region is encoded by SEQ ID NO: 9;
or
(c) the heavy chain variable region is encoded by SEQ ID NO: 21 and the light chain variable region is encoded by SEQ ID NO: 17;
or
(d) the heavy chain variable region is encoded by SEQ ID NO: 29 and the light chain variable region is encoded by SEQ ID NO: 25;
or
(e) the heavy chain variable region is encoded by SEQ ID NO: 33 and the light chain variable region is encoded by SEQ ID NO: 37;
or
(f) the heavy chain variable region is encoded by SEQ ID NO: 41 and the light chain variable region is encoded by SEQ ID NO: 45;
or
(g) the heavy chain variable region is encoded by SEQ ID NO: 49 and the light chain variable region is encoded by SEQ ID NO: 53;
or
(h) the heavy chain variable region is encoded by SEQ ID NO: 61 and the light chain variable region is encoded by SEQ ID NO: 57;
or
(i) the heavy chain variable region is encoded by SEQ ID NO: 69 and the light chain variable region is encoded by SEQ ID NO: 65;
or
(j) the heavy chain variable region is encoded by SEQ ID NO: 74 and the light chain variable region is encoded by SEQ ID NO: 72;
or
(k) the heavy chain variable region is encoded by SEQ ID NO: 77 and the light chain variable region is encoded by SEQ ID NO: 81;
or
(l) the heavy chain variable region is encoded by SEQ ID NO: 85 and the light chain variable region is encoded by SEQ ID NO: 89;
or
(m) the heavy chain variable region is encoded by SEQ ID NO: 92 and the light chain variable region is encoded by SEQ ID NO: 96;
or
(n) the heavy chain variable region is encoded by SEQ ID NO: 100 and the light chain variable region is encoded by SEQ ID NO: 104;
or
(o) the heavy chain variable region is encoded by SEQ ID NO: 110 and the light chain variable region is encoded by SEQ ID NO: 108;
or
(p) the heavy chain variable region is encoded by SEQ ID NO: 113 and the light chain variable region is encoded by SEQ ID NO: 116;
or
(q) the heavy chain variable region is encoded by SEQ ID NO: 122 and the light chain variable region is encoded by SEQ ID NO: 118;
or
(r) the heavy chain variable region is encoded by SEQ ID NO: 125 and the light chain variable region is encoded by SEQ ID NO: 313;
or
(s) the heavy chain variable region is encoded by SEQ ID NO: 135 and the light chain variable region is encoded by SEQ ID NO: 131;
or
(t) the heavy chain variable region is encoded by SEQ ID NO: 138 and the light chain variable region is encoded by SEQ ID NO: 142;
or
(u) the heavy chain variable region is encoded by SEQ ID NO: 148 and the light chain variable region is encoded by SEQ ID NO: 145;
or
(v) the heavy chain variable region is encoded by SEQ ID NO: 151 and the light chain variable region is encoded by SEQ ID NO: 155;
or
(w) the heavy chain variable region is encoded by SEQ ID NO: 163 and the light chain variable region is encoded by SEQ ID NO: 159;
or
(x) the heavy chain variable region is encoded by SEQ ID NO: 167 and the light chain variable region is encoded by SEQ ID NO: 170;
or
(y) the heavy chain variable region is encoded by SEQ ID NO: 175 the light chain variable region is encoded by SEQ ID NO: 173;
or
(z) the heavy chain variable region is encoded by SEQ ID NO: 182 the light chain variable region is encoded by SEQ ID NO: 179;
or
(aa) the heavy chain variable region is encoded by SEQ ID NO: 185 the light chain variable region is encoded by SEQ ID NO: 189;
or
(bb) the heavy chain variable region is encoded by SEQ ID NO: 192 the light chain variable region is encoded by SEQ ID NO: 196;
or
(cc) the heavy chain variable region is encoded by SEQ ID NO: 204 the light chain variable region is encoded by SEQ ID NO: 200;
or
(dd) the heavy chain variable region is encoded by SEQ ID NO: 209 the light chain variable region is encoded by SEQ ID NO: 159;
or
(ee) the heavy chain variable region is encoded by SEQ ID NO: 211 the light chain variable region is encoded by SEQ ID NO: 214.

3. The antibody or antigen binding fragment thereof of claim 1 or claim 2, wherein
(a) a heavy chain variable domain comprising a CDRH1 region consisting of SEQ ID NO: 42, a CDRH2 region consisting of SEQ ID NO: 74, and a CDRH3 region consisting of SEQ ID NO: 4; and a light chain variable domain comprising a CDRL1 region consisting of SEQ ID NO: 6, a CDRL2 region consisting of SEQ ID NO: 7, and a CDRL3 region consisting of SEQ ID NO: 8;
or
(b) a heavy chain variable domain comprising a CDRH1 region consisting of SEQ ID NO: 14, a CDRH2 region consisting of SEQ ID NO: 15, and a CDRH3 region consisting of SEQ ID NO: 16; and a light chain variable domain comprising a CDRL1 region consisting of SEQ ID NO: 10, a CDRL2 region consisting of SEQ ID NO: 11, and a CDRL3 region consisting of SEQ ID NO: 12;
or
(c) a heavy chain variable domain comprising a CDRH1 region consisting of SEQ ID NO: 22, a CDRH2 region consisting of SEQ ID NO: 23, and a CDRH3 region consisting of SEQ ID NO: 14; and a light chain variable domain comprising a CDRL1 region consisting of SEQ ID NO: 18, a CDRL2 region consisting of SEQ ID NO: 11, and a CDRL3 region consisting of SEQ ID NO: 20;
or
(d) a heavy chain variable domain comprising a CDRH1 region consisting of SEQ ID NO: 30, a CDRH2 region consisting of SEQ ID NO: 31, and a CDRH3 region consisting of SEQ ID NO: 32; and a light chain variable domain comprising a CDRL1 region consisting of SEQ ID NO: 26, a CDRL2 region consisting of SEQ ID NO: 27, and a CDRL3 region consisting of SEQ ID NO: 28;
or
(e) a heavy chain variable domain comprising a CDRH1 region consisting of SEQ ID NO: 78, a CDRH2 region consisting of SEQ ID NO: 35, and a CDRH3 region consisting of SEQ ID NO: 36; and a light chain variable domain comprising a CDRL1 region consisting of SEQ ID NO: 37, a CDRL2 region consisting of SEQ ID NO: 38, and a CDRL3 region consisting of SEQ ID NO: 39;
or
(f) a heavy chain variable domain comprising a CDRH1 region consisting of SEQ ID NO: 42, a CDRH2 region consisting of SEQ ID NO: 43, and a CDRH3 region consisting of SEQ ID NO: 44; and a light chain variable domain comprising a CDRL1 region consisting of SEQ ID NO: 46, a CDRL2 region consisting of SEQ ID NO: 47, and a CDRL3 region consisting of SEQ ID NO: 48;
or
(g) a heavy chain variable domain comprising a CDRH1 region consisting of SEQ ID NO: 50, a CDRH2 region consisting of SEQ ID NO: 51, and a CDRH3 region consisting of SEQ ID NO: 52; and a light chain variable domain comprising a CDRL1 region consisting of SEQ ID NO: 54, a CDRL2 region consisting of SEQ ID NO: 55, and a CDRL3 region consisting of SEQ ID NO: 56;
or
(h) a heavy chain variable domain comprising a CDRH1 region consisting of SEQ ID NO: 62, a CDRH2 region consisting of SEQ ID NO: 63, and a CDRH3 region consisting of SEQ ID NO: 64; and a light chain variable domain comprising a CDRL1 region consisting of SEQ ID NO: 58, a CDRL2 region consisting of SEQ ID NO: 59, and a CDRL3 region consisting of SEQ ID NO: 60;
or
(i) a heavy chain variable domain comprising a CDRH1 region consisting of SEQ ID NO: 42, a CDRH2 region consisting of SEQ ID NO: 70, and a CDRH3 region consisting of SEQ ID NO: 71; and a light chain variable domain comprising a CDRL1 region consisting of SEQ ID NO: 66, a CDRL2 region consisting of SEQ ID NO: 67, and a CDRL3 region consisting of SEQ ID NO: 68;
or
(j) a heavy chain variable domain comprising a CDRH1 region consisting of SEQ ID NO: 42, a CDRH2 region consisting of SEQ ID NO: 75, and a CDRH3 region consisting of SEQ ID NO: 76; and a light chain variable domain comprising a CDRL1 region consisting of SEQ ID NO: 38, a CDRL2 region consisting of SEQ ID NO: 39, and a CDRL3 region consisting of SEQ ID NO: 73;
or
(k) a heavy chain variable domain comprising a CDRH1 region consisting of SEQ ID NO: 78, a CDRH2 region consisting of SEQ ID NO: 79, and a CDRH3 region consisting of SEQ ID NO: 80; and a light chain variable domain comprising a CDRL1 region consisting of SEQ ID NO: 82, a CDRL2 region consisting of SEQ ID NO: 83, and a CDRL3 region consisting of SEQ ID NO: 84;
or
(l) a heavy chain variable domain comprising a CDRH1 region consisting of SEQ ID NO: 86, a CDRH2 region consisting of SEQ ID NO: 87, and a CDRH3 region consisting of SEQ ID NO: 88; and a light chain variable domain comprising a CDRL1 region consisting of SEQ ID NO: 82, a CDRL2 region consisting of SEQ ID NO: 83, and a CDRL3 region consisting of SEQ ID NO: 84;
or
(m) a heavy chain variable domain comprising a CDRH1 region consisting of SEQ ID NO: 93, a CDRH2 region consisting of SEQ ID NO: 94, and a CDRH3 region consisting of SEQ ID NO: 95; and a light chain variable domain comprising a CDRL1 region consisting of SEQ ID NO: 97, a CDRL2 region consisting of SEQ ID NO: 98, and a CDRL3 region consisting of SEQ ID NO: 99;
or
(n) a heavy chain variable domain comprising a CDRH1 region consisting of SEQ ID NO: 101, a CDRH2 region consisting of SEQ ID NO: 102, and a CDRH3 region consisting of SEQ ID NO: 103; and a light chain variable domain comprising a CDRL1 region consisting of SEQ ID NO: 105, a CDRL2 region consisting of SEQ ID NO: 106, and a CDRL3 region consisting of SEQ ID NO: 107;
or
(o) a heavy chain variable domain comprising a CDRH1 region consisting of SEQ ID NO: 42, a CDRH2 region consisting of SEQ ID NO: 111, and a CDRH3 region consisting of SEQ ID NO: 112; and a light chain variable domain comprising a CDRL1 region consisting of SEQ ID NO: 46, a CDRL2 region consisting of SEQ ID NO: 67, and a CDRL3 region consisting of SEQ ID NO: 109;
or
(p) a heavy chain variable domain comprising a CDRH1 region consisting of SEQ ID NO: 2, a CDRH2 region consisting of SEQ ID NO: 114, and a CDRH3 region consisting of SEQ ID NO: 115; and a light chain variable domain comprising a CDRL1 region consisting of SEQ ID NO: 46, a CDRL2 region consisting of SEQ ID NO: 47, and a CDRL3 region consisting of SEQ ID NO: 117;
or
(q) a heavy chain variable domain comprising a CDRH1 region consisting of SEQ ID NO: 123, a CDRH2 region consisting of SEQ ID NO: 75, and a CDRH3 region consisting of SEQ ID NO: 124; and a light chain variable domain comprising a CDRL1 region consisting of SEQ ID NO: 119, a CDRL2 region consisting of SEQ ID NO: 120, and a CDRL3 region consisting of SEQ ID NO: 121;
or
(r) a heavy chain variable domain comprising a CDRH1 region consisting of SEQ ID NO: 126, a CDRH2 region consisting of SEQ ID NO: 127, and a CDRH3 region consisting of SEQ ID NO: 128; and a light chain variable domain comprising a CDRL1 region consisting of SEQ ID NO: 129, a CDRL2 region consisting of SEQ ID NO: 39, and a CDRL3 region consisting of SEQ ID NO: 130;
or
(s) a heavy chain variable domain comprising a CDRH1 region consisting of SEQ ID NO: 78, a CDRH2 region consisting of SEQ ID NO: 136, and a CDRH3 region consisting of SEQ ID NO: 137; and a light chain variable domain comprising a CDRL1 region consisting of SEQ ID NO: 132, a CDRL2 region consisting of SEQ ID NO: 133, and a CDRL3 region consisting of SEQ ID NO: 134;
or
(t) a heavy chain variable domain comprising a CDRH1 region consisting of SEQ ID NO: 139, a CDRH2 region consisting of SEQ ID NO: 140, and a CDRH3 region consisting of SEQ ID NO: 141; and a light chain variable domain comprising a CDRL1 region consisting of SEQ ID NO: 143, a CDRL2 region consisting of SEQ ID NO: 67, and a CDRL3 region consisting of SEQ ID NO: 144;
or
(u) a heavy chain variable domain comprising a CDRH1 region consisting of SEQ ID NO: 149, a CDRH2 region consisting of SEQ ID NO: 75, and a CDRH3 region consisting of SEQ ID NO: 150; and a light chain variable domain comprising a CDRL1 region consisting of SEQ ID NO: 146, a CDRL2 region consisting of SEQ ID NO: 11, and a CDRL3 region consisting of SEQ ID NO: 147;
or
(v) a heavy chain variable domain comprising a CDRH1 region consisting of SEQ ID NO: 152, a CDRH2 region consisting of SEQ ID NO: 153, and a CDRH3 region consisting of SEQ ID NO: 154; and a light chain variable domain comprising a CDRL1 region consisting of SEQ ID NO: 156, a CDRL2 region consisting of SEQ ID NO: 157, and a CDRL3 region consisting of SEQ ID NO: 158;
or
(w) a heavy chain variable domain comprising a CDRH1 region consisting of SEQ ID NO: 164, a CDRH2 region consisting of SEQ ID NO: 165, and a CDRH3 region consisting of SEQ ID NO: 166; and a light chain variable domain comprising a CDRL1 region consisting of SEQ ID NO: 160, a CDRL2 region consisting of SEQ ID NO: 161, and a CDRL3 region consisting of SEQ ID NO: 162;
or
(x) a heavy chain variable domain comprising a CDRH1 region consisting of SEQ ID NO: 164, a CDRH2 region consisting of SEQ ID NO: 168, and a CDRH3 region consisting of SEQ ID NO: 169; and a light chain variable domain comprising a CDRL1 region consisting of SEQ ID NO: 171, a CDRL2 region consisting of SEQ ID NO: 7, and a CDRL3 region consisting of SEQ ID NO: 172;
or
(y) a heavy chain variable domain comprising a CDRH1 region consisting of SEQ ID NO: 176, a CDRH2 region consisting of SEQ ID NO: 177, and a CDRH3 region consisting of SEQ ID NO: 178; and a light chain variable domain comprising a CDRL1 region consisting of SEQ ID NO: 38, a CDRL2 region consisting of SEQ ID NO: 39, and a CDRL3 region consisting of SEQ ID NO: 174;
or
(z) a heavy chain variable domain comprising a CDRH1 region consisting of SEQ ID NO: 42, a CDRH2 region consisting of SEQ ID NO: 183, and a CDRH3 region consisting of SEQ ID NO: 184; and a light chain variable domain comprising a CDRL1 region consisting of SEQ ID NO: 180, a CDRL2 region consisting of SEQ ID NO: 39, and a CDRL3 region consisting of SEQ ID NO: 181;
or
(aa) a heavy chain variable domain comprising a CDRH1 region consisting of SEQ ID NO: 186, a CDRH2 region consisting of SEQ ID NO: 187, and a CDRH3 region consisting of SEQ ID NO: 188; and a light chain variable domain comprising a CDRL1 region consisting of SEQ ID NO: 190, a CDRL2 region consisting of SEQ ID NO: 106, and a CDRL3 region consisting of SEQ ID NO: 191;
or
(bb) a heavy chain variable domain comprising a CDRH1 region consisting of SEQ ID NO: 42, a CDRH2 region consisting of SEQ ID NO: 193, and a CDRH3 region consisting of SEQ ID NO: 194; and a light chain variable domain comprising a CDRL1 region consisting of SEQ ID NO: 197, a CDRL2 region consisting of SEQ ID NO: 198, and a CDRL3 region consisting of SEQ ID NO: 199;
or
(cc) a heavy chain variable domain comprising a CDRH1 region consisting of SEQ ID NO: 205, a CDRH2 region consisting of SEQ ID NO: 206, and a CDRH3 region consisting of SEQ ID NO: 207; and a light chain variable domain comprising a CDRL1 region consisting of SEQ ID NO: 201, a CDRL2 region consisting of SEQ ID NO: 202, and a CDRL3 region consisting of SEQ ID NO: 203;
or
(dd) a heavy chain variable domain comprising a CDRH1 region consisting of SEQ ID NO: 164, a CDRH2 region consisting of SEQ ID NO: 165, and a CDRH3 region consisting of SEQ ID NO: 210; and a light chain variable domain comprising a CDRL1 region consisting of SEQ ID NO: 160, a CDRL2 region consisting of SEQ ID NO: 208, and a CDRL3 region consisting of SEQ ID NO: 162;
or
(ee) a heavy chain variable domain comprising a CDRH1 region consisting of SEQ ID NO: 42, a CDRH2 region consisting of SEQ ID NO: 212, and a CDRH3 region consisting of SEQ ID NO: 213; and a light chain variable domain comprising a CDRL1 region consisting of SEQ ID NO: 215, a CDRL2 region consisting of SEQ ID NO: 47, and a CDRL3 region consisting of SEQ ID NO: 216.

4. The antibody or antigen binding fragment thereof of any of claims 1-3, wherein the antibody is a chimeric antibody and the heavy chain constant domain is from rabbit, mouse, rat, or nonhuman primate.

5. The antibody or antigen binding fragment thereof of any of claims 1-4, wherein the light chain constant domain is a kappa light chain constant domain or a lambda light chain constant domain.

6. The antibody or antigen binding fragment thereof of any of claims 1-5, wherein the antibody is linked to a detectable label.

7. A peptide comprising SEQ ID NO: 281 or a peptide comprising a sequence with 95% similarity to SEQ ID NO: 281.

8. The peptide of claim 7, further comprising a detectable label.

9. The peptide of claim 7 or 8, wherein the peptide is linked to a solid support.

10. A method of diagnosing Kawasaki Disease in a subject, comprising the steps of:
i) obtaining a sample from a subject suspected of having Kawasaki Disease;
ii) contacting the sample with the antibody or antigen binding fragment thereof of any of claims 1-3; and
iii) detecting the binding of the antibody to a component of the sample, whereby binding of the antibody to the component of the sample indicates the presence of Kawasaki Disease and confirms the diagnosis of Kawasaki disease in the subject.

11. The method of claim 10, further comprising
iv) treating the subject diagnosed with Kawasaki disease with intravenous immunoglobulin (IV Ig).

12. The method of claim 10 or claim 11, wherein the sample is a blood sample or a serum sample.

13. The method of any of claims 10-12, wherein detecting the binding of the antibody or antigen binding fragment thereof in the sample is carried out using ELISA, Western blot, immunostaining, immunoprecipitation, flow cytometry, sensor chips, or magnetic beads.

14. The method of any of claims 10-13, wherein the antibody or antigen binding fragment thereof is linked to a solid support.

15. The method of claim 14, wherein detecting the binding of the antibody to the component of the sample comprises contacting the sample with an antibody or antigen fragment thereof of any of claims 1-3.

16. A method of detecting intracytoplasmic inclusion bodies in a subject, comprising the steps of:
i) obtaining a sample from a subject suspected of having Kawasaki Disease;
ii) contacting the sample with the antibody or antigen binding fragment thereof of any of claims 1-3; and
iii) detecting the binding of the antibody or antigen binding fragment thereof in the sample, whereby binding of the antibody indicates the presence of intracytoplasmic inclusion bodies as compared to a negative control.

17. The method of claim 16, further comprising
iv) treating the subject having detected antibodies associated with Kawasaki disease with intravenous immunoglobulin (IV Ig).

18. A method of detecting antibodies associated with Kawasaki disease in a subject comprising the steps of:
i) obtaining a sample comprising antibodies from a subject suspected of having Kawasaki disease;
ii) contacting the sample with the peptide of any of claims 7-9; and
iii) detecting the binding of antibodies to the peptide to form a peptide-antibody complex, wherein the presence of a peptide-antibody complex confirms the presence of antibodies associated with Kawasaki disease in the subject.

19. The method of claim 18, further comprising
iv) treating the subject having detected antibodies associated with Kawasaki disease with intravenous immunoglobulin (IV Ig).

20. The method of claim 18 or 19, wherein the peptide is linked to a solid support.

21. The method of any of claims 18-20, wherein detecting comprises contacting the peptide-antibody complex with a secondary antibody wherein the secondary antibody is optionally linked to a detectable label.

22. The method of claim 21, wherein the secondary antibody is an anti-human Fc antibody.

23. A kit comprising:
i) the antibody or antigen binding fragment thereof of any of claims 1-3;
ii) a detection reagent.

24. The kit of claim 23, further comprising:
iii) a solid support.

25. The kit of claim 24, wherein the antibody or antigen binding fragment thereof is linked to the solid support.

26. The kit of claim 24 or 25, wherein the solid support comprises a lateral flow device.

27. The kit of any of claims 23-26, wherein the detection reagent comprises an antibody or antigen binding fragment thereof of any of claims 1-3.

28. A kit comprising:
i) a peptide of any of claims 7-9;
ii) a detection reagent.

29. The kit of claim 28, further comprising:
iii) a solid support,
wherein the peptide is linked to the solid support.

30. The kit of claim 29, wherein the detection reagent comprises a secondary antibody optionally linked to a detectable label.
